(19) United States
(12) Patent Application Publication
(10) Pub. No.: US 2026/0308586 A1
(43) Pub. Date: Oct. 8, 2026

(54) APOE-ACTIVATING NONCODING RNA (eRNA) AND USES THEREOF

(71) Applicants: Robert J. MAHLEY, San Francisco, CA (US); Jason WATTS, San Francisco, CA (US); Vivian G. CHEUNG, San Francisco, CA (US); The J. David Gladstone Institutes, a testamentary trust established under the Will of J. David, San Francisco, CA (US)

(72) Inventors: Robert W. Mahley, San Francisco, CA (US); Jason Watts; Vivian G. Cheung

(21) Appl. No.: 19/480,540

(22) PCT Filed: May 1, 2024
(86) PCT No.: PCT/US2024/027272

§ 371 Date: Oct. 31, 2025

Related U.S. Application Data
(60) Provisional application No. 63/463,165, filed on May 1, 2023.

(51) Int. Cl. C12N 15/113; A61P 25/28

(52) U.S. Cl. CPC C12N 15/113; A61P 25/28; C12N 2310/14

(57) ABSTRACT

Provided herein are compositions and methods for turning off the APOE valve for APOE expression, by targeting the non-coding RNA AANCR.

### PRIORITY

This application claims the benefit of priority of U.S. provisional patent application No. 63/463,165, filed on May 1, 2023, the benefit of priority of which is claimed hereby, and which is incorporated by reference herein in its entirety.

### GOVERNMENT SUPPORT

This invention was made with government support under AG059751, ES03491901 and ES103361 awarded by the National Institutes of Health. The government has certain rights in the invention.

### BACKGROUND

RNA is modified by hundreds of chemical reactions and folds into innumerable shapes. However, the regulatory role of RNA sequence and structure and how dysregulation leads to diseases remain largely unknown.

### SUMMARY

Provided herein is an enhancer RNA (eRNA), named APOE-activating noncoding RNA, (AANCR) and uses thereof. AANCR regulates the transcription and expression of the gene Apolipoprotein E (APOE). APOE is a genetic risk factor for Alzheimer's Disease (AD); about two-thirds of Alzheimer's Disease patients have at least one copy of the APOE4 allele. It is demonstrated herein that AANCR regulates the transcription and expression of APOE in human cells at baseline and in response to stress. It is further shown that AANCR and APOE expression levels are correlated. Knockdown of AANCR by siRNA or antisense oligonucleotides leads to a decrease in APOE expression. Furthermore, the suppression of AANCR transcription also leads to lower APOE expression. The eRNA, AANCR, is a therapeutic target of Alzheimer's Disease; by targeting it one can suppress the expression of APOE. This therapeutic is particularly relevant in individuals with one or two copies of APOE4, who are at risk for developing AD.

AD is a progressive neurological disease characterized by dementia. In the United States, there are more than 5 million AD patients, and the cost of caring for the patients exceeds $300 billion annually. The APOE4 variant of apolipoprotein E is a major genetic risk factor for developing AD. Approximately two-thirds of AD patients have at least one copy of the APOE4 allele. The risk effect of APOE4 on AD is consistent with a dominant-like inheritance with moderate penetrance.

The region around APOE harbors genetic modifiers for AD. Sequence variants in the region around APOE seem to affect APOE expression, which in turn influences AD risk conferred by APOE4. A noncoding RNA upstream of APOE was identified that acts as a genetic modifier of AD. This eRNA, AANCR, regulates APOE expression and modifies the effect of APOE4.

AANCR is an eRNA that Regulates APOE Expression.

AANCR is a capped, nonpolyadenylated noncoding RNA found between TOMM40 and APOE. BLASTP, PFAM analysis, and analyses using several algorithms that test for coding potentials showed that AANCR is noncoding. In cells that produce APOE, such as HepG2 and astrocytes, AANCR is expressed as a full-length noncoding RNA that activates APOE expression. Whereas in cells such as fibroblasts and B-cells that do not express APOE, AANCR is only partially transcribed and does not activate APOE expression.

In iPSC-derived neurons, microglia cells and astrocytes, knockdown of AANCR decreases APOE expression significantly (P<0.001); with 50% knockdown of AANCR, gene and proteins expression of APOE decreased by >75%.

The region where AANCR resides is characterized by DNase I hypersensitivity, H3K27ac, and H3K4me1 which are hallmarks of enhancer chromatin. With HiC analysis, it was shown that AANCR and APOE are in the same chromatin-associated domain but TOMM40 is in a separate domain; thus, AANCR is an enhancer of APOE4 but not TOMM40.

Using GTEx data, it was found that genetic variants, including rs405509 in AANCR, are significantly associated with APOE expression, including in the hippocampus. Thus, molecular and genetic data show that AANCR, a noncoding RNA, is an enhancer of APOE expression.

### AANCR is a Modifier of Alzheimer's Disease.

Given that AANCR regulates APOE expression and APOE4 is a risk factor for AD, it was asked if polymorphisms in AANCR affect susceptibility to AD. GWA studies show that SNPs in AANCR are significantly associated with AD (Table 1). AANCR is located upstream of APOE, between TOMM40 and APOE. Despite the proximity to APOE, the extent of linkage disequilibrium between AANCR and APOE is quite modest. Together, the results show that polymorphisms in AANCR are genetic factors (in addition to APOE) that influence susceptibility to AD. Thus, AANCR is a modifier that regulates APOE expression and affects AD risk.

TABLE 1
SNP alleles in AANCR are significantly
associated with Alzheimer's disease.

| SNP ID | P-value | Study |
|---|---|---|
| rs449647 | 2 × 10⁻⁵⁸ | Up to 42,034 British ancestry individuals |
| | | with parental history of Alzheimer's disease |
| | 8.7 × 10⁻¹⁸ | Transethnic LOAD |
| | 2.6 × 10⁻³¹ | Meta-analysis of 74,046 individuals |
| rs405509 | 4 × 10⁻¹⁴ | 952 European ancestry male cases, 1,789 |
| | (females) | European ancestry female cases |
| | 1 × 10⁻⁸ | |
| | (males) | |
| | 3 × 10⁻⁴⁴ | ADGC GWAS Stage 1 |
| | 6.1 × 10⁻¹² | ADGC African American |

### APOE Expression and Alzheimer's Disease.

Alternation in gene expression may be a component of neurodegeneration. Studies have found that APOE is expressed at higher levels in AD patients and that APOE gene expression is higher in the hippocampus of Alzheimer's patients compared to controls. Additionally, APOE expression is correlated with progression of cognitive decline. Many studies have demonstrated that APOE expression affects amyloid accumulation, and depletion of APOE has been shown to improve pathology. In mouse models, lowering APOE levels also decreases pathologic amyloid accumulation. However, in contrast, some studies showed that lower APOE levels are associated with AD. These contrasting findings suggest that there is not a simple high or low expression of APOE that explains AD, rather the effect may be different based on APOE genotype or cell types and/or conditions.

### Stress, APOE, and Alzheimer's Disease.

Oxidative stress damage and neuroinflammation play significant roles in pathogenesis of AD. Given the high oxygen consumption of the brain, it is highly vulnerable to oxidative damage. Mitochondrial dysfunction, inflammation, toxic metals, and AB peptides contribute to oxidation of lipids, proteins, and nucleic acids. As neurons are stressed, they accumulate damage ultimately setting up a vicious cycle of oxidative damage, inflammation, and ultimately cell death.

Studies have associated APOE and oxidative damage including a recent mechanistic link between neuronal APOE expression, MHC-1 expression, tau pathology, and neurodegeneration. It was found that APOE is stress responsive in human cells: the cell types that do not express APOE at baseline induce APOE expression under stress. It was further shown that condition media with more abundant APOE decreases stress-induced cell death.

One embodiment provides a method to modulate expression of APOE in a cell comprising increasing or decreasing expression of APOE-activating noncoding RNA (AANCR) in said cells to increase or decrease APOE expression.

In one embodiment, expression of AANCR is decreased.

In one embodiment, expression AANCR is decreased by treating said cell with an AANCR inhibitory nucleic acid and/or a small molecule inhibitor, and correspondingly decrease APOE expression, including APOE4 expression.

In one embodiment, the inhibitory nucleic acid is an dsRNA, siRNA, shRNA, miRNA, ami-RNA, antisense oligonucleotide, gapmer antisense oligonucleotide, or an RNA aptamer. In one embodiment, the inhibitory nucleic acid binds AANCR. In one embodiment, the inhibitory nucleic acid binds anywhere within AANCR, such as within the 5′ portion, middle or 3′ portion of AANCR, such as within the first 950 nucleic acids, of the DNA or RNA that codes for AANCR. Further, the inhibitory nucleic acid could target the 5′ region where FOS and Jun bind, i.e., AP-1 site. In one embodiment, the inhibitory nucleic acid binds within the following sequence GCGTGGCCTGGGGTCGCTATCTTCCCATCCGGAACATCTGCCCTGCTGGG GGACACTACGGGCCTTCCCTTGCCTGAGGGTAGGGTCTCAAGGTCACTTG CCCCCAGCTTGACCTGGCCGGAGTGGCTATAGAGGACTTTGTCCCTGCAG ACTGCAGCAGCAGAGATGACACTGTCTCTGAGTGCAGAGATGGGGGCAGG GAGCTGGGAGAGGGTTCAAGCTACTGGAACAGCTTCAGAACAACTAGGGT ACTAGGAACTGCTGTGTCAGGGAGAAGGGGCTCAAGGACTCGCAGGCCTG GGAGGAGGGGCCTAGGCCA (SEQ ID NO: 46) or the corresponding transcribed eRNA sequence. In one embodiment, the siRNAs are (individually or in combination)

siAANCR #1
Sense:
(SEQ IDN NO: 47)
CCAUGCUCAAUACACGUUAtt
Antisense:
(SEQ IDN NO: 48)
UAACGUGUAUUGAGCAUGGag,
siAANCR #2
Sense:
(SEQ IDN NO: 49)
GGAUUUAUAACAGGGCUUAtt
Antisense:
(SEQ IDN NO: 50)
UAAGCCCUGUUAUAAAUCCca,
siAANCR #3
Sense:
(SEQ IDN NO: 51)
UCCUAACCUUAACCCAGAAtt
Antisense:
(SEQ IDN NO: 52
UUCUGGGUUAAGGUUAGGAtt,
siRNA #4
Antisense
(SEQ IDN NO: 53)
CTGTCTCTGAGTGCAGAGATG

or at least 90% identity thereto.

In one embodiment, APOE4 is also knocked down.

In one embodiment, the small molecule inhibitor stabilizes the R-loop in AANCR, induces an RNA abasic site in the R-loop in AANCR, pauses the RNA Pol II transcription of AANCR and/or inhibits AANCR-APOE interaction. In another embodiment, the small molecule inhibitor comprises SR11302, TPA, STC-15 (ejcancer.com/action/showPdf?pii=S0959-8049%2822%2901128-5) or METTL3/14 inhibitors (STM2457) that inhibit full-length transcription of AANCR. One embodiment provides for the use of compounds that decrease AP-1 to decrease AANCR or compounds that increase AP-1 to increase AANCR.

The decrease of AANCR expression by RNA modalities or small molecules inhibitors can be done in conjunction with APOE4 inhibition by RNA modalities to specifically decrease APOE4 expression, for example, for those individuals who are susceptible to AD because of their APOE4 genotypes.

In one embodiment, expression of AANCR is increased.

In one embodiment, expression of AANCR is increased by contacting said cell with AANCR RNA, an expression vector coding for AANCR RNA or exposing the said cell to stress (such as environmental stress). In one embodiment, expression of AANCR is increased by exposing said cell to stress, and correspondingly APOE expression is also increased. One embodiment provides a method to protect a cell against apoptosis during stress comprising contacting said cell with AANCR RNA, an expression vector coding for AANCR RNA or stress. In one embodiment, the stress is osmotic, amyloid beta aggregate exposure, mitochondrial stress (such as decouplers (antimycin)), heavy metal and/or heat.

In one embodiment, said cell is an astrocyte, microglia, oligodendrocyte, macrophage, endothelial, T-cell, B cell, fibroblast, renal cell, hepatocyte or neuron. In one embodiment, the renal cell is a proximal tubule cell or a collecting duct cell.

Methods of treating diseases and/or disorders are provided herein.

One embodiment provides a method to treat Alzheimer's Disease in a subject in need thereof comprising administering to said subject an AANCR inhibitory nucleic acid or a small molecule that inhibits full AANCR transcription. In one embodiment, the subject has one or two copies of APOE4.

Another embodiment provides a method to treat brain injury/disease or kidney disease in a subject in need thereof comprising administering to said subject an AANCR inhibitory nucleic acid or a small molecule that inhibits full AANCR transcription. In one embodiment, the brain injury/disease is stroke, Parkinson's, caused or aggravated by viral infection (APOE genotype has been documented to severity of HIV, COVID, hepatitis C, herpes simplex and/or influenza) or cardiovascular disease (AANCR-APOE2).

In one embodiment, the inhibitory nucleic acid is an dsRNA, siRNA, shRNA, miRNA, ami-RNA, antisense oligonucleotide, gapmer antisense oligonucleotide, or an RNA aptamer. In another embodiment, the inhibitory nucleic acid binds AANCR. In one embodiment, the inhibitory nucleic acid binds within the 5′ portion, such as within the first 950 nucleic acids, of the DNA or RNA that codes for AANCR. In one embodiment, the siRNA comprises (individually or in combination)

siAANCR #1
Sense:
(SEQ IDN NO: 47)
CCAUGCUCAAUACACGUUAtt
Antisense:
(SEQ IDN NO: 48)
UAACGUGUAUUGAGCAUGGag,
siAANCR #2
Sense:
(SEQ IDN NO: 49)
GGAUUUAUAACAGGGCUUAtt
Antisense:
(SEQ IDN NO: 50)
UAAGCCCUGUUAUAAAUCCca,
siAANCR #3
Sense:
(SEQ IDN NO: 51)
UCCUAACCUUAACCCAGAAtt
Antisense:
(SEQ IDN NO: 52)
UUCUGGGUUAAGGUUAGGAtt,
siRNA #4
Antisense
(SEQ IDN NO: 53)
CTGTCTCTGAGTGCAGAGATG

or at least 90% identity thereto.

In one embodiment, the small molecule inhibitor stabilizes the R-loop in AANCR, induces an RNA abasic site in R-loop in AANCR, pauses the RNA Pol II transcription of AANCR and/or inhibits AANCR-APOE interaction.

One embodiment provides a method to treat cell stress in a subject in need thereof comprising administering to said subject AANCR RNA or an expression vector coding for AANCR RNA. In one embodiment, the stress is osmotic cell stress. In one embodiment, the subject has osmotic demyelination syndrome.

In one embodiment, administration is by catheter, surgical placement, or injection, subcutaneous, intranasal administration or taken orally.

### BRIEF DESCRIPTION OF THE DRAWINGS

FIGS. 1A-ID. APOE is regulated by methylpurine glycosylase (MPG). (A) Representative RNA-seq showing APOE expression in fibroblasts before and after MPG knockdown (days 3 and 7), Y-axis is RPM. (N=2). (B) Immunoblot of MPG and APOE expression before and after MPG knockdown (day 7). GAPDH is a loading control (N>5). (C) Densitometry quantification of immunoblots of secreted APOE protein before and after MPG knockdown (day 7) (N=3, P<<0.001, t-test, error bars=S.E.M.). NTC is the non-target control. (D) PRO-seq results for APOE before and after MPG knockdown (day 7) are plotted. The bar at each nucleotide location represents the abundance of RNA Pol II. APOE transcription is induced after MPG knockdown. Y-axis is RPM (N=2).

FIGS. 2A-2C. Upstream R-loops regulate APOE expression. (A) Average S9.6 DNA-RNA hybrid immunoprecipitation (DRIP)-seq data in fibroblasts (N=5) are plotted (top panel) and show R-loops upstream (5′) of APOE. Average PRO-seq results in fibroblasts (N=5) are plotted (bottom panel), showing active transcription in the intergenic region upstream of APOE, and RNA Pol II pausing upstream of the R-loops (pausing index=7.5). Y-axis is RPM. Arrows indicate qPCR primers used in subsequent figures. Genomic coordinates (hg19) are indicated. (B) Data from Negative Elongation Factor Complex Member A (NELFA) chromatin immunoprecipitation followed by qPCR with primers indicated by arrows in (A) are shown as fold enrichment over IgG in arbitrary units (a.u.) (N=2, error bars are S.E.M). (C) APOE expression levels in fibroblasts from family-control and amyotrophic lateral sclerosis type 4 (ALS4) patients are plotted, each dot represents the APOE expression of one person (*P<0.05, t-test).

FIGS. 3A-3E. Upstream of APOE, RNA abasic sites stabilize R-loops. (A) Data from S9.6 DRIP, MPG RNA-IP, and ARP RNA-pulldown followed by PCR are shown as fold enrichment compared to input in arbitrary units (a.u.). Primers corresponding to the R-loops are shown in FIG. 2A. S9.6 DRIP-PCR confirmed the location of the R-loops (N=3,***P<0.001, t-test, error bars=S.E.M.), MPG RIP-PCR shows binding of MPG to RNA of the R-loops (N=3, ****P<0.0001; t-test, error bars=S.E.M.), and ARP RNA-pulldown-PCR shows abasic sites in the RNA of the R-loops (N=2, ***P<0.001; t-test, error bars=S.E.M.). (B) R-loop abundance measured by S9.6 dot blot (see Materials and Methods) shows a dose-dependent increase of R-loops at 24 h following treatment with estrogen (P<0.0001; ANOVA, error bars=S.E.M.) and time-dependent increase following 100 nM estrogen treatment (P<0.05; ANOVA, error bars=S.E.M.). (C) RNA abasic site abundance as measured by ARP-labeling shows a dose (N=3, ****P<0.0001; ANOVA, error bars=S.E.M.) and time (N=3, *P<0.05; ANOVA, error bars=S.E.M.) dependent increase following treatment with estrogen. Y-axis is fold-enrichment relative to control. (D) R-loop abundance was measured by S9.6 dot-blot in fibroblasts treated with scrambled siRNA (nontarget control, NTC) and siRNA against MPG, the cells were then given estrogen (0-100 nM) (N=2, error bars=S.E.M.). R-loops do not accumulate in response to estrogen when MPG is knocked down. (E) More R-loops are present upstream of APOE as measured by S9.6 DRIP-PCR in fibroblasts followed by estrogen treatment (100 mM). Location of the assessed R-loops is shown in the schematic and corresponds to the R loops in FIG. 2A (N=3; ****P<0.0001, t-test, error bars=S.E.M.).

FIGS. 4A-4E. N6-Methyladenosine (m6A) is the precursor to RNA abasic site. (A) m6A in the R-loops upstream of APOE are identified by m6A-RIP followed by PCR with primers shown in the schematic and corresponds to the R-loops in FIG. 2A (N=3, ***P<0.001; t-test, error bars=S.E.M.). m6A enrichment is shown as fold-over input in arbitrary units (a.u). (B) fewer R-loops upstream of APOE (N=3, ****P<0.0001; t-test, error bars=S.E.M.) and (C) higher APOE expression levels (N=3, *P<0.05; t-test, error bars=S.E.M.) in cells treated with siRNA targeting N6-Methyladenosine methyltransferase (METTL3). Y-axis is fold-expression relative to non-target control (NTC). (D) MPG expression in fibroblast with and without METTL3 siRNA knockdown as measured by RT-PCR (N=2, P>0.5, error bars=S.E.M.). (E) MPG binding to m6ARNA/DNA hybrids (top; Kd=460±22 nM), RNA/m6ADNA hybrids (middle; Kd=562±51 nM), or unmodified RNA/DNA hybrids (bottom; Kd>>661±89) was measured by gel shift and were fitted (see Materials and Methods) to the modified Hill equation (N≥3, error bars=S.D.).

FIGS. 5A-5I. AANCR functions as an R-loop-dependent enhancer. (A) CoPRO identified capped RNA in the intergenic region upstream of APOE, the cap coincides with nascent transcription from PRO-seq. The transcription start site of the non-coding RNA, AANCR, is marked based on the cap location. Genomic coordinates (hg19) are indicated. (B) RNA-seq data showing full-length AANCR in iPSC-derived hepatocytes and a partial transcript of AANCR in white blood cells (WBC). BRU-seq data show full-length AANCR in HepG2 liver cells and partial AANCR transcript in cultured B-cells. (C, D) At the AANCR locus, ChIP-seq results show enhancer marks H3K27ac and H3K4me1, and DNase mapping results show DNase hypersensitivity in fibroblasts (C) and HepG2 cells (D). (E) Expression of full-length AANCR (N=3, P<0.05; t-test, error bars=S.E.M.) and APOE (N=3, **P<0.01; t-test, error bars=S.E.M.) are induced in response to hypertonic stress with the addition of 50 mM NaCl (400 mOsm final) to the culture media of HK-2 renal proximal tubule cells. (F) Representative immunoblot showing APOE expression in HK-2 cells following addition of 50 mM NaCl (N=2). (G) Immunoblot showing increased APOE secretion into culture media following hypertonic stress (N=1). (H) Luminescent detection of Annexin V signal in arbitrary units (a.u.) following hypertonic stress. HK-2 cells cultured in hypertonic conditioned media with more APOE protein, have less apoptosis than cells cultured in hypertonic media with less APOE protein (N=3; P<0.0001, ANOVA, error bars=S.E.M.). (I) Schematic of the SNPs in AANCR and APOE.

FIGS. 6A-6E. R-loop with RNA modifications pause elongating RNA Pol II. (A) Heatmaps of m6A modified nascent RNA, MPG RIP-seq results, and PROseq (27) at 1,966 non-coding RNAs with paused RNA Pol II in the gene body in B-cells. Average signals for m6A (gold), MPG (orange), and PRO-seq (blue) are plotted. Heatmap signals are scaled to maximal signal per row in arbitrary units. The transcripts are in the same order across panels with AACNR located on row 637. (B) Elongating RNA Pol II pauses in three cell types. PRO-seq reads for three representative transcripts are plotted for B-cells, fibroblasts, and HK-2 proximal tubule cells. Genomic locations are hg19. Y-axis is reads per million. (C) Average ChIP seq results (black line) show NELFA 5_to paused RNA Pol II (PRO-seq data, blue line), with R-loop (DRIP-seq, red line) 3 of paused RNA Pol II. (D and E) Average H3K27ac and H3K4me1 ChIP-seq results indicate these are enhancer RNA. (C-E) Data from fibroblasts are shown for the same 1,966 noncoding transcripts as shown in panel A. PRO-seq results are scaled as in panel A and ChIP-seq data are scaled to reads per million, with error bands=S.E.M.

FIG. 7. A model where AANCR with an R-loop with abasic sites does not induce APOE. AANCR without an R-loop allows for full length expression and activation of APOE (Watts et al. 2022).

FIG. 8. AANCR knockdown: decrease is APOE expression. Knockdown of AANCR by siRNA or gapmer antisense oligonucleotides results in downregulation of APOE expression. This confirms targeting AANCR can reduce APOE expression.

FIG. 9. APOE is involved in general stress response. Treating cells with amyloid beta aggregates, the aggregate that forms in AD, results in increase AANCR expression and increased APOE expression. Similar for oxidative phosphorylation inhibitor (mitochondrial stress) antimycin C.

FIG. 10. ATM mediates APOE stress response. In B-cells with ATM mutations subject to osmotic stress, it has been shown that signaling downstream of the DNA damage response protein ATM induces the stress response.

FIG. 11. AANCR and APOE: not activated in cells with ATM mutations. In cells from Caucasians of European descent (CEU) with two normal copies of ATM, there is increased expression of the tonicity-responsive transcription factor NFAT5 and AANCR and APOE. But in cells with one (AT het) or two (AT pt) copies of mutant ATM, there is no induction of NFAT5, AANCR, or APOE. Thus, ATM is an upstream regulator of AANCR.

FIG. 12. NFAT5 does not bind AANCR. But MAP kinase 8 is downstream of ATM and activates JUN and FOS. JUN-FOS heterodimers form the transcription factor AP-1. AP-1 could bind and activate AANCR.

FIG. 13. AP-1 regulates AANCR and APOE expression in B-cells. Treating B-cells with AP-1 inhibitor (SR11302; MedChem Express) represses both AANCR and APOE expression. Treating with an AP-1 activator (TPA; cell signaling) induces AANCR and APOE. Treating cells with hypertonic salt (50 mM NaCl) and the AP-1 inhibitor represses the stress responsive gene expression, which can be rescued with the AP-1 activator, TPA.

FIG. 14. AP-1 regulates AANCR and APOE expression in resting astrocytes. Treating astrocytes with AP-1 inhibitor (SR11302) represses both AANCR and APOE expression. Treating with an AP-1 activator (TPA) induces AANCR and APOE. Treating cells with hypertonic salt and the AP-1 inhibitor represses the stress responsive gene expression, whereas hypertonic salt with the AP-1 activator leads to greater induction than hypertonic salt alone.

FIG. 15. Concentrated HK-2 media protects against apoptosis. Another example of the effect of conditioned media on cell viability following stress. Media from cells was collected and protein concentrator was used to create a fraction without APOE or a fraction with high concentration of APOE. Compared to regular media, there is more apoptosis in cells in hypertonic media without APOE (orange line). Whereas cells exposed to hypertonic media with concentrated APOE (blue line) are protected against cell death. This suggests increased APOE can be protective in conditions of osmotic cell stress.

FIGS. 16A-16B. APOE4 and APOE3 differ by one nucleotide (A). Method to turn off the valve for APOE expression, by targeting the non-coding RNA AANCR (B).

FIGS. 17A-17E. Knockdown of the enhancer RNA, AANCR, decreases APOE gene and protein expression in astrocytes. AANCR is knocked down by RNA interference with two separate siRNAs and as a pool of the two siRNAs. The expression levels of AANCR (A) and APOE (B) decreased significantly after silencing the AANCR. AANCR expression decreased following knocked down with an antisense oligonucleotide (C), correspondingly the APOE expression decreased (D) and the amount of secreted APOE protein (E) was reduced significantly (n≥3, error bar=S.E.M. *=P<0.05, **=P<0.005).

FIGS. 18A-18C. Knockdown of the enhancer RNA, AANCR, decreases APOE gene expression in microglia and iPSC-induced neurons. AANCR and APOE are knocked down with siAANCR (pool), siAPOE (pool) and combined siAANCRs and siAPOEs. The expression level of AANCR decreased significantly following AANCR but not APOE silencing (A). APOE expression decreased after AANCR and APOE knockdown as well as combined AANCR and APOE knockdown (B). The expression of AANCR decreased following siAANCR (pool) and correspondingly APOE expression also decreased significantly in iPSC-derived neurons. (n≥3, error bar=S.E.M. NS=not significant, *=P<0.05, **=P<0.005, ***=P<0.001).

FIGS. 19A-19C. The colocalization of AANCR and APOE increases in response to stress. Single molecule fluorescent in situ hybridization was carried out to assess the interaction of AANCR and APOE in the nuclei of single cells. (A) Fraction of HK-2 proximal tubule nuclei with AANCR-APOE colocation after osmotic stress is significantly higher (P<0.0001; one-sided ANOVA; n=3) after osmotic stress. (B) Fluorescent smiFISH images of AANCR (top, red) and APOE (middle, green) RNA in astrocytes following osmotic stress. In the merged images (bottom), arrows indicate AANCR-APOE colocalization (yellow). DAPI nuclear stain in blue. Scale bar XXX m (C) Fraction of astrocyte nuclei with AANCR-APOE colocation after osmotic stress is significantly higher (P<0.001; one-sided ANOVA; n≥2) after osmotic stress. In the experiments, 900-1300 nuclei were quantified per sample.

FIGS. 20A-20G. Antimycin A and Aβ42 aggregates induce AANCR and APOE expression in microglia and B-cells. In microglia treated with antimycin A, AANCR (A) and APOE (B) expression increased in a time-dependent manner. In microglia, AANCR (C) and APOE (D) expression levels increased in response to Aβ42 aggregates. In peripheral B-cells, AANCR (E) and APOE (F) expression levels also increased in response to Aβ42 aggregates (n≥3, error bar=S.E.M. *=P<0.05, **=P<0.005, ***=P<0.001).

FIGS. 21A-21D. Paraquat and Aβ42 aggregates induce AANCR and APOE expression in astrocytes. In astrocytes, AANCR (A) and APOE (B) expression levels increased significantly following paraquat treatment. AANCR and APOE expression levels also increased in response to Aβ42 aggregates. (n≥3, error bar=S.E.M. *=P<0.05, **=P<0.005, ***=P<0.001)

FIGS. 22A-22G. AP-1 regulates AANCR and therefore APOE expression in astrocytes. Data from the ENCODE consortium showing JUN and FOSL1 that constitute AP-1 bind to AANCR (A). CRISPR-mediated mutation of the AP-1 site in AANCR results in a decrease in JUN occupancy as found by JUN ChTP assay (N=2, P<0.05; t-test) (B). In astrocytes, SR11302 which inhibits AP-1 decreases AANCR (C) and APOE expression (D), whereas TPA, an activator of AP-1 increases AANCR (C) and APOE expression (D). siRNA against the AP-1 site of AANCR decreases AANCR and APOE expression (E). In astrocytes, osmotic stress increases AANCR (F) and APOE (G) expression, and this induction can be 5 suppressed by SR11302 and rescued with TPA (F and G). (for C-G, n≥3, error bar=S.E.M. *=P<0.05, **=P<0.005, ***=P<0.001)

FIGS. 23A-23G. ATM-ERK-JUN regulates AANCR expression. In response to osmotic stress, B cells from controls induces AANCR and APOE expression while those from AT-patients with ATM null mutations induces significantly less AANCR and APOE (A and B). Data from each individual are shown as a dot. In microglia, ATM is induced within 15 mins of antimycin A treatment; representative western blot (n=3) is shown in (C). Following ATM induction, ERK is phosphorylated and subsequently JUN is phosphorylated (E) in microglia treated with antimycin A; pERK and pJUN were measured by immunblots. The phosphorylation of JUN was inhibited by treatment with SP600125 (F) and in microglia where phosphorylation of JUN is inhibited, AANCR is induced at a significantly lower level (G). (n≥3, error bar=S.E.M. *=P<0.05, **=P<0.005, ***=P<0.001).

FIGS. 24A-24F. AANCR and APOE knockdown confers a less inflammatory expression phenotype. A correlation matrix with genes whose expression levels changed significantly in astrocytes with AANCR and APOE knockdown; data based on cDNA-sequencing of the samples (A). The expression of complement genes, including C3, decreased significantly after AANCR or APOE knockdown (B). The expression of genes including S100A10 that are markers of A2 astrocytes increased significantly after AANCR or APOE knockdown (C). The expression of RELA decreased significantly after AANCR or APOE knockdown (D). Genes that changed significantly following AANCR and APOE knockdown include genes that encode proteins that interact with RIG-1 per data in Biogrid. A network of their interactions as determined by STRING using the confidence model for edges (E). Expression levels of MIF and UNC93B1 are reduced following AANCR and APOE knockdown (F). (n=5 siAANCR experiments, n=5 siAPOE experiments, error bar=S.E.M.)

FIGS. 25A-25C. AANCR and APOE knockdown leads to a reduction in mitochondrial function. Heat map showing expression of 32 mitochondrial-related genes downregulated by knockdown of AANCR and APOE (A). Seahorse assay of astrocytes after AANCR knockdown. The oxygen consumption rate (OCR) (B) and extracellular acidification rate (C) are significantly lower (n=3 per treatment, error bars=XX, P<0.0001; one-sided ANOVA for B&C).

FIGS. S1A-S1B. AP-1 regulates AANCR and APOE response to osmotic stress in B-cells. In Bcells, SR11302 that inhibits AP-1 decreases AANCR (A) and APOE expression (B), whereas TPA, an activator of AP-1 increases AANCR (A) and APOE expression (B). (n=4, error bar=S.E.M. *=P<0.05, **=P<0.005, ***=P<0.001)

FIG. S2. Validation of AANCR knockdown in astrocytes used for gene expression analysis. In astrocytes, siRNAs and ASO significantly reduced AANCR (A) and APOE (B) expression.

### DESCRIPTION OF THE INVENTION

RNA is modified by hundreds of chemical reactions and folds into innumerable shapes. However, the regulatory role of RNA sequence and structure and how dysregulation leads to diseases remain largely unknown. Provided herein is a mechanism wherein RNA abasic sites in R-loops regulate transcription by pausing RNA polymerase II. Also provided herein is an enhancer RNA, AANCR, that regulates the transcription and expression of apolipoprotein E (APOE). In some human cells, such as fibroblasts, AANCR is folded into an R-loop and modified by N-glycosidic cleavage; in this form, AANCR is a partially transcribed nonfunctional enhancer and APOE is not expressed. In contrast, in other cell types including hepatocytes and those under stress, AANCR does not form a stable R-loop as its sequence is not modified, so it is transcribed into a full-length enhancer that promotes APOE expression. DNA sequence variants in AANCR are associated significantly with APOE expression and Alzheimer's Disease, thus AANCR is a modifier of Alzheimer's Disease. Besides AANCR, thousands of noncoding RNAs are regulated by abasic sites in R-loops. Together the data reveals the folding and modification of RNA in cellular regulation and demonstrate that dysregulation underlies common complex diseases such as Alzheimer's disease.

The cellular control of APOE expression through modulation of AANCR provides a mechanism whereby one can decrease the expression of APOE in the context of Alzheimer's disease pathology.

### Definitions

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, several embodiments with regards to methods and materials are described herein. As used herein, each of the following terms has the meaning associated with it in this section.

For the purposes of clarity and a concise description, features can be described herein as part of the same or separate embodiments; however, it will be appreciated that the scope of the invention may include embodiments having combinations of all or some of the features described.

References in the specification to “one embodiment,” “an embodiment,” etc., indicate that the embodiment described may include a particular aspect, feature, structure, moiety, or characteristic, but not every embodiment necessarily includes that aspect, feature, structure, moiety, or characteristic. Moreover, such phrases may, but do not necessarily, refer to the same embodiment referred to in other portions of the specification. Further, when a particular aspect, feature, structure, moiety, or characteristic is described in connection with an embodiment, it is within the knowledge of one skilled in the art to affect or connect such aspect, feature, structure, moiety, or characteristic with other embodiments, whether or not explicitly described.

As used herein, the indefinite articles “a,” “an” and “the” should be understood to include plural reference unless the context clearly indicates otherwise.

The phrase “and/or,” as used herein, should be understood to mean “either or both” of the elements so conjoined, e.g., elements that are conjunctively present in some cases and disjunctively present in other cases.

As used herein, “or” should be understood to have the same meaning as “and/or” as defined above. For example, when separating a listing of items, “and/or” or “or” shall be interpreted as being inclusive, e.g., the inclusion of at least one, but also including more than one of a number of items, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e., “one or the other but not both”) when preceded by terms of exclusivity, such as “either,” “one of,” “only one of,” or “exactly one of.”

As used herein, the terms “including,” “includes,” “having,” “has,” “with,” or variants thereof, are intended to be inclusive similar to the term “comprising.”

As used herein, the term “about” means plus or minus 10% of the indicated value. For example, about 100 means from 90 to 110. Numerical ranges recited herein by endpoints include all numbers and fractions subsumed within that range (e.g., 1 to 5 includes 1, 1.5, 2, 2.75, 3, 3.90, 4, and 5). It is also to be understood that all numbers and fractions thereof are presumed to be modified by the term “about.”

As used herein, the term “antisense oligonucleotide” or antisense nucleic acid means a nucleic acid polymer, at least a portion of which is complementary to a nucleic acid which is present in a cell. “Antisense” refers particularly to the nucleic acid sequence of the non-coding strand of a double stranded DNA molecule, or to a sequence which is substantially homologous to the non-coding strand. It is not necessary that the antisense sequence be complementary solely to the coding portion of the coding strand of the DNA molecule. The antisense sequence may be complementary to regulatory sequences specified on the coding strand of a DNA molecule encoding a protein, which regulatory sequences control expression of the coding sequences. The antisense oligonucleotides of the invention include, but are not limited to, phosphorothioate oligonucleotides and other modifications of oligonucleotides.

A “vector” is a composition of matter which comprises an isolated nucleic acid and which can be used to deliver the isolated nucleic acid to the interior of a cell. Numerous vectors are known in the art including, but not limited to, linear polynucleotides, polynucleotides associated with ionic or amphiphilic compounds, plasmids, and viruses. Thus, the term “vector” includes an autonomously replicating plasmid or a virus. The term should also be construed to include non-plasmid and non-viral compounds which facilitate transfer or delivery of nucleic acid to cells, such as, for example, polylysine compounds, liposomes, and the like. Examples of viral vectors include, but are not limited to, adenoviral vectors, adeno-associated virus vectors, retroviral vectors, recombinant viral vectors, and the like. Examples of non-viral vectors include, but are not limited to, liposomes, polyamine derivatives of DNA and the like.

“Expression vector” refers to a vector comprising a recombinant polynucleotide comprising expression control sequences operatively linked to a nucleotide sequence to be expressed. An expression vector comprises sufficient cis-acting elements for expression; other elements for expression can be supplied by the host cell or in an in vitro expression system. Expression vectors include all those known in the art, such as cosmids, plasmids (e.g., naked or contained in liposomes) and viruses that incorporate the recombinant polynucleotide. “Complementary” refers to the broad concept of sequence complementarity between regions of two nucleic acid strands or between two regions of the same nucleic acid strand. It is known that an adenine residue of a first nucleic acid region is capable of forming specific hydrogen bonds (“base pairing”) with a residue of a second nucleic acid region which is antiparallel to the first region if the residue is thymine or uracil. As used herein, the terms “complementary” or “complementarity” are used in reference to polynucleotides (i.e., a sequence of nucleotides) related by the base pairing rules. For example, for the sequence “A G T,” is complementary to the sequence “T C A.”

Similarly, it is known that a cytosine residue of a first nucleic acid strand is capable of base pairing with a residue of a second nucleic acid strand which is antiparallel to the first strand if the residue is guanine. A first region of a nucleic acid is complementary to a second region of the same or a different nucleic acid if, when the two regions are arranged in an antiparallel fashion, at least one nucleotide residue of the first region is capable of base pairing with a residue of the second region. Preferably, the first region comprises a first portion and the second region comprises a second portion, whereby, when the first and second portions are arranged in an antiparallel fashion, at least about 50%, and at least about 75%, at least about 90%, or at least about 95% of the nucleotide residues of the first portion are capable of base pairing with nucleotide residues in the second portion. More preferably, all nucleotide residues of the first portion are capable of base pairing with nucleotide residues in the second portion.

“Homologous” or “identity” as used herein, refers to the subunit sequence similarity between two polymeric molecules, e.g., between two nucleic acid molecules, e.g., two DNA molecules or two RNA molecules, or between two polypeptide molecules. When a subunit position in both of the two molecules is occupied by the same monomeric subunit, e.g., if a position in each of two DNA molecules is occupied by adenine, then they are homologous at that position. The homology between two sequences is a direct function of the number of matching or homologous positions, e.g., if half (e.g., five positions in a polymer ten subunits in length) of the positions in two compound sequences are homologous then the two sequences are 50% homologous, if 90% of the positions, e.g., 9 of 10, are matched or homologous, the two sequences share 90% homology. By way of example, the DNA sequences 3′ATTGCC5′ and 3′TATGGC share 50% homology.

As used herein, “homology” is used synonymously with “identity.”

The determination of percent identity between two nucleotide sequences can be accomplished using a mathematical algorithm. For example, a mathematical algorithm useful for comparing two sequences is the algorithm of Karlin and Altschul (1990, Proc. Natl. Acad. Sci. USA 87:2264-2268), modified as in Karlin and Altschul (1993, Proc. Natl. Acad. Sci. USA 90:5873-5877). This algorithm is incorporated into the NBLAST and XBLAST programs of Altschul, et al. (1990, J. Mol. Biol. 215:403-410), and can be accessed, for example at the National Center for Biotechnology Information (NCBI) world wide web site. BLAST nucleotide searches can be performed with the NBLAST program (designated “blastn” at the NCBI web site), using the following parameters: gap penalty=5; gap extension penalty=2; mismatch penalty=3; match reward=1; expectation value 10.0; and word size=11 to obtain nucleotide sequences homologous to a nucleic acid described herein. BLAST protein searches can be performed with the XBLAST program (designated “blastn” at the NCBI web site) or the NCBI “blastp” program, using the following parameters: expectation value 10.0, BLOSUM62 scoring matrix to obtain amino acid sequences homologous to a protein molecule described herein. To obtain gapped alignments for comparison purposes, Gapped BLAST can be utilized as described in Altschul et al. (1997, Nucleic Acids Res. 25:3389-3402). Alternatively, PSI-Blast or PHI-Blast can be used to perform an iterated search which detects distant relationships between molecules (Id.) and relationships between molecules which share a common pattern. When utilizing BLAST, Gapped BLAST, PSI-Blast, and PHI-Blast programs, the default parameters of the respective programs (e.g., XBLAST and NBLAST) can be used.

The percent identity between two sequences can be determined using techniques similar to those described above, with or without allowing gaps. In calculating percent identity, typically exact matches are counted.

“Substantially homologous nucleic acid sequence” means a nucleic acid sequence corresponding to a reference nucleic acid sequence wherein the corresponding sequence encodes a peptide having substantially the same structure and function as the peptide encoded by the reference nucleic acid sequence; e.g., where only changes in amino acids not significantly affecting the peptide function occur. Preferably, the substantially similar nucleic acid sequence encodes the peptide encoded by the reference nucleic acid sequence. The percentage of identity between the substantially similar nucleic acid sequence and the reference nucleic acid sequence is at least about 50%, 65%, 75%, 85%, 95%, 96%, 97%, 98%, 99% or more.

As use herein, the terms “administration of” and or “administering” should be understood to mean providing means to increase or decrease AANCR expression (e.g., RNA, DNA, protein or small molecule) to a subject in need of treatment.

As used herein, an “effective amount” means an amount sufficient to produce a selected effect, such as alleviating symptoms of a disease or disorder. The term to “treat,” as used herein, means reducing the frequency with which symptoms are experienced by a patient or subject or administering an agent or compound to reduce the frequency with which symptoms are experienced.

The term “delivery vehicle” or “carrier” refers to any kind of device or material which can be used to deliver compounds in vivo or can be added to a composition comprising compounds administered to a plant or animal. This includes, but is not limited to, implantable devices, aggregates of cells, matrix materials, gels, nucleic acids, proteins etc.

A “preventive” or “prophylactic” treatment is a treatment administered to a subject who does not exhibit signs, or exhibits only early signs, of a disease or disorder. A prophylactic or preventative treatment is administered for the purpose of decreasing the risk of developing pathology associated with developing the disease or disorder.

As used herein “injecting, administering or applying” includes administration of the invention by any number of routes and means including, but not limited to, topical, oral, buccal, intravenous, intramuscular, intra-arterial, intramedullary, intrathecal, intraventricular, transdermal, subcutaneous, intraperitoneal, intranasal, enteral, topical, sublingual, vaginal, ophthalmic, pulmonary, or rectal means.

As used herein, a “subject in need thereof” is a patient, animal (domestic (cat, dog) or farm animal (livestock, horse, cow), mammal, or human, who will benefit from the method of this invention.

A disease, condition, or disorder is “alleviated” if the severity of a symptom of the disease or disorder, the frequency with which such a symptom is experienced by a subject, or both, are reduced.

The term “inhibit,” as used herein, refers to the ability of a compound, agent, or method to reduce or impede a described function, level, activity, rate, etc., based on the context in which the term “inhibit” is used. For example, inhibition is by at least 10%, by at least 25%, by at least 50%, or the function is inhibited by at least 75%. The term “inhibit” is used interchangeably with “reduce” and “block.” As used herein, an “instructional material” includes a publication, a recording, a diagram, or any other medium of expression which can be used to communicate the usefulness of the of the invention in the kit for effecting alleviation of the various diseases or disorders recited herein. Optionally, or alternately, the instructional material may describe one or more methods of alleviating the diseases or disorders in a cell or a tissue of a mammal. The instructional material of the kit of the invention may, for example, be affixed to a container which contains the identified invention or be shipped together with a container. Alternatively, the instructional material may be shipped separately from the container with the intention that the instructional material and the invention be used cooperatively by the recipient.

As used herein, the terms “including”, “includes”, “having”, “has”, “with”, or variants thereof, are intended to be inclusive similar to the term “comprising.”

The terms “comprises,” “comprising,” and the like can have the meaning ascribed to them in U.S. Patent Law and can mean “includes,” “including” and the like. As used herein, “including” or “includes” or the like means including, without limitation.

### I. AANCR

The eRNA, AANCR regulates the transcription and expression of the gene Apolipoprotein E (APOE). Provided below is the human sequence of the eRNA AANCR (SEQ ID NO: 1)

GAAAAAAAACGAUGGGAGGCCUCCGUUUUCUCAAGUGUGUCUGGCCUGU
UUUGAGCAUUUCAUCCGGAGUCUGGCCGCCCUGACCUUCCCCCAGCCGC
CUGCAGGGGGCGCCAGAGGGCCGGAGCACGGAAAGCAGCGGAUCCUUGA
UGCUGCCUUAAGUCCGGCUCAGAGGGGCGCAGCGUGGCCUGGGGUCGCU
AUCUUCCCAUCCGGAACAUCUGCCCUGCUGGGGGACACUACGGGCCUUC
CCUUGCCUGAGGGUAGGGUCUCAAGGUCACUUGCCCCCAGCUUGACCUG
GCCGGAGUGGCUAUAGAGGACUUUGUCCCUGCAGACUGCAGCAGCAGAG
AUGACACUGUCUCUGAGUGCAGAGAUGGGGGCAGGGAGCUGGGAGAGGG
UUCAAGCUACUGGAACAGCUUCAGAACAACUAGGGUACUAGGAACUGCU
GUGUCAGGGAGAAGGGGCUCAAGGACUCGCAGGCCUGGGAGGAGGGGCC
UAGGCCAGCCAUGGGAGUUGGGUCACCUGUGUCUGAGGACUUGGUGCUG
UCUGGAUUUUGCCAACCUAGGGCUGGGGUCAGCUGAUGCCCACCACGAC
UCCCGAGCCUCCAGGAACUGAAACCCUGUCUGCCCCCAGGGUCUGGGGA
AGGAGGCUGCUGAGUAGAACCAACCCCAGGUUACCAACCCCACCUCAGC
CACCCCUUGCCAGCCAAAGCAAACAGGCCCGGCCCGGCACUGGGGGUUC
CUUCUCGAACCAGGAGUUCAGCCUCCCCUGACCCGCAGAAUCUUCUGAU
CCCACCCGCUCCAGGAGCCAGGAAUGAGUCCCAGUCUCUCCCAGUUCUC
ACUGUGUGGUUUUGCCAUUCGUCUUGCUGCUGAACCACGGGUUUCUCCU
CUGAAACAUCUGGGAUUUAUAACAGGGCUUAGGAAAGUGACAGCGUCUG
AGCGUUCACUGUGGCCUGUCCAUUGCUAGCCCUAACAUAGGACCGCUGU
GUGCCAGGGCUGUCCUCCAUGCUCAAUACACGUUAGCUUGUCACCAAAC
AUACCCGUGCCGCUGCUUUCCCAGUCUGAUGAGCAAAGGAACUUGAUGC
UCAGAGAGGACAAGUCAUUUGCCCAAGGUCACACAGCUGGCAACUGGCA
GAGCCAGGAUUCACGCCCUGGCAAUUUGACUCCAGAAUCCUAACCUUAA
CCCAGAAGCACGGCUUCAAGCCCCUGGAAACCACAAUACCUGUGGCAGC
CAGGGGGAGGUGCUGGAAUCUCAUUUCACAUGUGGGGAGGGGGCUCCCC
UGUGCUCAAGGUCACAACCAAAGAGGAAGCUGUGAUUAAAACCCAGGUC
CCAUUUGCAAAGCCUCGACUUUUAGCAGGUGCAUCAUACUGUUCCCACC
CCUCCCAUCCCACUUCUGUCCAGCCGCCUAGCCCCACUUUCUUUUUUUU
CUUUUUUUGAGACAGUCUCCCUCUUGCUGAGGCUGGAGUGCAGUGGCGA
GAUCUCGGCUCACUGUAACCUCCGCCUCCCGGGUUCAAGCGAUUCUCCU
GCCUCAGCCUCCCAAGUAGCUAGGAUUACAGGCGCCCGCCACCACGCCU
GGCUAACUUUUGUAUUUUUAGUAGAGAUGGGGUUUCACCAUGUUGGCCA
GGCUGGUCUCAAACUCCUGACCUUAAGUGAUUCGCCCACUGUGGCCUCC
CAAAGUGCUGGGAUUACAGGCGUGAGCUACCGCCCCCAGCCCCUCCCAU
CCCACUUCUGUCCAGCCCCCUAGCCCUACUUUCUUUCUGGGAUCCAGGA
GUCCAGAUCCCCAGCCCCCUCUCCAGAUUACAUUCAUCCAGGCACAGGA
AAGGACAGGGUCAGGAAAGGAGGACUCUGGGCGGCAGCCUCCACAUUCC
CCUUCCACGCUUGGCCCCCAGAAUGGAGGAGGGUGUCUGUAUUACUGGG
CGAGGUGUCCUCCCUUCCUGGGGACUGUGGGGGGUGGUCAAAAGACCUC
UAUGCCCCACCUCCUUCCUCCCUCUGCCCUGCUGUGCCUGGGGCAGGGG
GAGAACAGCCCACCUCGUGACUGGGGGCUGGCCCAGCCCGCCCUAUCCC
UGGGGGAGGGGGCGGGACAGGGGGAGCCCUAUAAUUGGACAAGUCUGGG
AUCCUUGAGUC

The eRNA, AANCR regulates the transcription and expression of the gene Apolipoprotein E (APOE). Provided below is the mouse intergenic region sequence between TOMM40 and APOE corresponding to mm10 chr7:19699046-19701312. (SEQ ID NO: 2)

UCUGGGAGGCCUCGGUUUUCUCCAGUUGUUGACAUGGUUGCCAGCAGGCG
GCGCCGAGGGUUCAGAAAGCACAGCAGCACCAAGCAGUUUAGAGAAAGCU
UGGCCAGGCAUGGUCACACCCUCUGCUCUUGGGGACUUACACUGCCGGAA
CAUCAGAGGCCUGGCCUCACCAGGAGGGUGGCUCCAGGUCACUCGCCUCU
GUGCAGUCUGGCCCCGAGAGCUGGCAGAGGACUUUGUCUCUGUAAACAGG
GUGGGGGCAGGGAGACGGGGCUCAGGAGCCUCCCUGUCCCAAAACGGGCU
GAGGUGGUAGCUUGUGCUGACUUUCUCCCAGUGGGAAGGUCAGAGGUCUC
AGAAACUUCAGGAAGAAACGGAGUUCCUGGAAGUUCAGCACAAUAAGGAA
AGUUACUCUGGGAGGAACGGGCCCUAGCACCUUCCCAGGCUGAAACAGGA
AAUGUCAAGUUGUUUUGUUUUUUAAAUUUUGUUGAAGAUGUAGCUCUGGG
CCUCACGUGUCAGGCAGGUGCUGCUGGGUUCCCUAUAGUGCUUUUCCGUG
AGGCCUGCUCUGGGGUGUGCGAUAAGGGCCUCAGAUUGGGCUCUGCAUCU
CACUGCUGCACCUCAUGGCAUCCCAAGGGAAGCAAAGACUCACGAUGAAC
UGGUCAGUGUCCUAGACCGCAGCACUAAGACCCUCUCUACUGCCUGGGCU
GGAGAUGGGAGCUGGCCCUUAGCAACCCAUGAAAAUCAUCUCACCACACC
CAGUCCUUGCCAGUGUUUUCUGAAGCCAAAGCUAACAGGCCUGGGCCUGG
CCAGGCACCCUGUACGUACCCUUGGAGCCAGGUGUUCCGCCUCUGCCCAU
CCUGCAGAAUCAUGUUUUGCCGUGUCUGGUGCCAAACACUGCUAUGUGGC
UUCUCUCCUGCCAUCAACAGCUGGGAACAGGGAACCUUGUGCAGGCAGUG
CUUCUAGCAAGCUUGCUGUGGUCUCUGAGCCCCUUGUCCUACCUGACUUC
CCAGGUACAAUGGCUUUCCCACUUUUUGGGGGUUUUGUUGUUGUUGGGUU
UUUUUUUGUUUUUCAAGACAGGGUUUCUCUGUGUAGUCCUGUCGGUCCUG
GAACUCACUUUGUAGACCAGGCUGGCCUCCAACUCAGAAAUUCACCUGCC
UCUGCCUCCAAGUGCUGGGAUUAUGGGAUUAAAGGCAUGUGCCACCACGC
CCAGCUGGCUUUUCCACUUUUUAGCCAGGACUUCAUUCUAUUACCUGAGC
UCGGGAUCUUCCUGCCUCAGCUUUGCAUAUGGCUAGCACUAUAGACCCAU
GUUCCAGUGAAUGACUUAUGGCUUGUCUUUUUUUUUUUUUUUUUUUUUUU
UUAUGUGCAUUAGUGUUUUGCCUGCAUGUAUGCCUUCGUGAGGGUAGCAG
AUCUUGGUGUUACAGUUGUGAGCUGCUGUGUGGGUGCUGCGAUUUUGAAC
CUAGGUCCUGUGAAAUGCAGUCAGUGCUCCUAACCUCUGAGUCAUCUCUC
CAGCUCCUGCUCUUCUGCUUUUAUGAGGAAAAAGAAAAGAGAAGUGGCUU
GAGAGUGGAAAAUGCACAUGCAGGGGUGCACACCUGCAGUCCCAGCAUGC
UACAGCAGAGGCAGAAGGACCUUUGUGGGUUAGAGGGCAGCCUGAGAAUC
UUAUCUCAAAACAACUUUUUAAAAUGUGCUCUGUAGGGGUAGCUCUUCCC
UCCCAAGGUGACACAUCUGGCAAUCGCCAGAAACAGAUCAGGAGCAUCAA
CGCUUGGUUUCCCAGGGCUUGGCUUAAUGUAUGGCUUCAAACCCAUCGGG
AGCCACCACUGAACAGCUCCUGAAGGAACUGGAGCACGUCCCAGCCUUGG
AAUGGAAAGAGUUCACCUGUGGUGGAGGAAUCAACAACGAGGGAUCCCAG
AACAACGAUCUUCACCCCAGAAGCUGAGCCUCUUAGCCCCCACCCACCCA
UUUCCAUUUAGGCUGCCAGCUCUUUUCUUUACAAUGCACCAGACCCCGCG
GGGAAAGGGAAGGAGCGGUUCUCAGUGCCCCAGUACCAAGGCCUGGAUUA
UUCAAUGAGGUGUCCGCUCCCUUUGUUGGCGGGGGAGGGGAGCGGGGGGU
CACAAGGCAUCCAAACUCCACCUCUUUCCUCUGCCCUGCUGUGAAGGGGG
AGAGAACAACCCGCCUCGUGACAGGGGGCUGGCACAGCCCGCCCUAGCCC
UGAGGAGGGGGCGGGACAGGGGGAGUCCUAUAAUUGGACCGGUCUGGGAU
CCGAUCCCCUGCUCAGA

### A. Inhibitors of AANCR

Inhibitors of AANCR include, but are not limited to, inhibitory nucleic acids and small molecule inhibitors. Inhibitory nucleic acids include, but are not limited to, dsRNA, siRNA, shRNA, miRNA, ami-RNA, antisense oligonucleotide, gapmer antisense oligonucleotide, or an RNA aptamer. Example of inhibitory nucleic acids include, but are not limited to,

siAANCR #1
Sense:
(SEQ IDN NO: 47)
CCAUGCUCAAUACACGUUAtt
Antisense:
(SEQ IDN NO: 48)
UAACGUGUAUUGAGCAUGGag
siAANCR #2
Sense:
(SEQ IDN NO: 49)
GGAUUUAUAACAGGGCUUAtt
Antisense:
(SEQ IDN NO: 50)
UAAGCCCUGUUAUAAAUCCca
siAANCR #3
Sense:
(SEQ IDN NO: 51)
UCCUAACCUUAACCCAGAAtt
Antisense:
(SEQ IDN NO: 52)
UUCUGGGUUAAGGUUAGGAtt
siRNA AP1
Antisense
(SEQ IDN NO: 53)
CTGTCTCTGAGTGCAGAGATG

One of skill in the art would be able to readily design and generate other inhibitory nucleic acids based on the sequence of AANCR provides herein with methods available to an art worker.

Inhibition of AANCR can also include CRISPR-based editing or base editing.

### B. Increase AANCR

The amount of AANCR can be increased by contacting cells/administering AANCR RNA or vectors, as discussed above that, express AANCR.

### II. Diseases/Conditions

In one aspect, diseases and/or conditions that can be treated by the invention provided herein are those wherein APOE plays a role in the disease and/or condition, such Alzheimer's Disease (AD), brain injury or kidney disease and/or cell stress, such osmotic cell stress, including osmotic demyelination syndrome. Other diseases and/or conditions include, but are not limited to, stroke, Parkinson's, brain conditions caused or aggravated by viral infections (APOE genotype has been documented to severity of HIV, COVID, hepatitis C, herpes simplex and influenza) or cardiovascular disease (AANCR-APOE2).

### III. Administration

In some embodiments, AANCR RNA, DNA, expression vectors and/or small molecules are administered to a subject.

The administration can be local by, for example, injection or with the use of a catheter or during surgery. The administration can also be oral, intravenous or subcutaneous.

The RNA (including, but not limiting to, mRNA, siRNA, ASO, RNA aptamer and/or saRNA) can be linear (similar to that used in the COVID-19 vaccines).

The nucleic acids, vectors or small molecules can be present in a pharmaceutically acceptable carrier. As used herein, the term “pharmaceutically-acceptable carrier” means a chemical composition that can be combined with the invention and which, following the combination, can be used to administer to a subject.

Pharmaceutically acceptable carriers include physiologically tolerable or acceptable diluents, excipients, solvents or adjuvants. The compositions are preferably sterile and nonpyrogenic. Examples of suitable carriers include, but are not limited to, water, normal saline, dextrose, mannitol, lactose or other sugars, lecithin, albumin, sodium glutamate, cysteine hydrochloride, ethanol, polyols (propylene glycol, polyethylene glycol, glycerol, and the like), vegetable oils (such as olive oil), injectable organic esters such as ethyl oleate, ethoxylated isosteraryl alcohols, polyoxyethylene sorbitol and sorbitan esters, microcrystalline cellulose, aluminum methahydroxide, bentonite, kaolin, agar-agar and tragacanth, lipids/liposomes, lipid nanoparticles or mixtures of these substances, and the like.

The pharmaceutical compositions may also contain minor amounts of nontoxic auxiliary pharmaceutical substances or excipients and/or additives, such as wetting agents, emulsifying agents, pH buffering agents, antibacterial and antifungal agents (such as parabens, chlorobutanol, phenol, sorbic acid, and the like). Suitable additives include, but are not limited to, physiologically biocompatible buffers (e.g., tromethamine hydrochloride), additions (e.g., 0.01 to 10 mole percent) of chelants (such as, for example, DTPA or DTPA-bisamide) or calcium chelate complexes (as for example calcium DTPA or CaNaDTPA-bisamide), or, optionally, additions (e.g. 1 to 50 mole percent) of calcium or sodium salts (for example, calcium chloride, calcium ascorbate, calcium gluconate or calcium lactate). If desired, absorption enhancing or delaying agents (such as liposomes, aluminum monostearate, or gelatin) may be used. The compositions can be prepared in conventional forms, either as liquid solutions or suspensions, solid forms suitable for solution or suspension in liquid prior to injection, or as emulsions. Pharmaceutical compositions according to the present invention can be prepared in a manner fully within the skill of the art.

### EXAMPLES

The following examples are provided in order to demonstrate and further illustrate certain embodiments and aspects of the present invention and are not to be construed as limiting the scope thereof.

### Example I—A Common Transcriptional Mechanism Involving R-Loop and RNA Abasic Site Regulates an Enhancer RNA of APOE

### Introduction

DNA is the genetic code while RNA is the regulatory code of all organisms. DNA sequence differences account for individual variation from gene expression (1,2) to disease susceptibility. While technological advances have made it easier to identify DNA sequence mutations and variants that account for the genetic bases of diseases, molecular understanding remains a challenge. To gain deeper insight, it is necessary to delve into both the genetic and regulatory codes. RNA sequence and structure comprise the regulatory code. RNA is composed of four canonical bases (A, C, G and U) and over 150 modified bases (3) that form a myriad of structures. In an organism, every cell has largely the same DNA, but different RNA species. RNA is what confers cell identity and function. It is templated from DNA, but during and after synthesis, it is highly processed by hundreds of chemical steps that modify the bases and sugar of RNA. Chemical modifications of RNA include methylation that generates N6-methyladenosine to multiple-step reactions that form wybutosine.

RNA sequence and structure are closely related; they regulate each other and co-regulate gene expression and function. RNA sequence affects its structure and conversely, its structure can further alter the sequence, as in RNA editing in which (adenosine-uridine) AU-rich sequences form stem-loop structures that are bound by adenosine deaminase RNA-specific (ADAR) proteins to deaminate adenosine to inosine (4,5). RNA sequence and structure also mediate the interaction between RNA and regulatory nucleic acids and proteins; for example, the repression of mRNA by microRNA and Argonaut-2 is dependent on both sequence and structure (6).

The interest of the inventors in RNA sequence and structure was deepened as they studied amyotrophic lateral sclerosis type 4, a juvenile-onset ALS due to heterozygous senataxin mutations (7). The patients have significantly fewer R-loops (8,9), three-stranded nucleic acid structures, each with an RNA/DNA hybrid, and a displaced single-stranded DNA (10-13). To understand how the deficiency of R-loops affects cell function, proteins that bind to these nucleic acid structures were sought out. Hundreds of R-loop binding proteins have been identified (14-16). These studies use different methods, yet the results consistently show the same set of several hundred proteins. These include a number of enzymes that modify nucleic acids such as METTL3 and METTL14 that methylate adenosine inRNA to form N6-methyladenosine, as well as MPG and apurinic/apyrimidinic endonuclease 1 (APE1) that were known to process DNA (17). These results suggest that R-loops serve as platforms for processing nucleic acid including RNA modification. Yet to confirm their regulatory roles, the mechanism must be determined for how each protein processes nucleic acid individually and jointly.

The focus was first on two proteins, MPG and APE1, and it was found that MPG not only cleaves the N-glycosidic bond on DNA but also on RNA, leading to RNA abasic sites (18). APE1 then processes the RNA by cleaving the sugar phosphate backbone at the abasic sites. The activity of MPG and APE1 on RNA occurs only when the RNA is hybridized to a DNA strand, as in an R-loop, thus further illustrating the co-dependence of sequence and structure. Mass spectrometry analysis shows that abasic sites are not rare inRNA; there are about four RNA abasic sites per million ribonucleotides in human cells such as primary fibroblasts (18) so there are hundreds of thousands of RNA abasic sites in a cell. Given their abundance, it is necessary to study them beyond knowing how they form.

Provided herein is the study of RNA abasic sites in R-loops and a gene regulatory mechanism by which RNA abasic sites stabilize R-loops to pause RNA Polymerase II transcription. A noncoding Enhancer RNA of apolipoprotein E (APOE) was found, which is referred to as APOE-activating noncoding RNA, AANCR, whose expression and function are regulated dynamically by pausing RNA Polymerase II. When this noncoding enhancer RNA is full-length, it activates APOE expression. When this RNA is only partially transcribed, it is nonfunctional and APOE is not expressed. In some cells, the noncoding AANCR RNA is not fully transcribed since RNA Polymerase II elongation is paused by R-loops that are stabilized by RNA abasic sites. In response to hypertonic stress, the R-loops that pause transcription resolve, and AANCR RNA is transcribed into a full-length enhancer that activates APOE expression. By genetic analysis, it is shown that sequence variants in the enhancer region affect APOE expression in several cell types, including the hippocampus, and are associated with Alzheimer's disease. Besides this noncoding enhancer RNA of APOE, transcription elongation of more than 1,000 other noncoding RNAs are regulated by R-loops with RNA abasic sites. Thus, herein is revealed a gene regulatory mechanism in which the sequences and structures of noncoding RNAs regulate their transcription, function, and consequently disease susceptibility.

### Materials and Methods

### Astrocyte Expression

Paired-end RNA-seq data from human astrocytes (19) were downloaded from the NCBI GEO database and aligned to the hg19 reference genome using STAR version 2.6.0c. Read coverage for AANCR and APOE were obtained using the samtools bedcov function on the aligned bam files. The reads for each gene were normalized by millions of reads for each sample (RPM). The hg19 chromosome coordinates used for AANCR were chr19:45406985-45408892 and for APOE were chr19:45409039-45412650.

### Cell Culture

Foreskin fibroblasts from healthy newborns were cultured in MEM medium (Thermo-Fisher, Cat #11095080) supplemented with 10% fetal bovine serum, 1% L glutamine, and 1% penicillin-streptomycin. HK-2 cells (ATCC, Cat #CRL-2190) were cultured in DMEM/F12 supplemented with 10% fetal bovine serum and 1% penicillin-streptomycin. A549 (ATCC, Cat #CCL-185) and HepG2 (ATCC, Cat #HB-8065) cells were cultured in DMEM supplemented with 10% fetal bovine serum and 1% penicillin-streptomycin. B-lymphoblasts (Coriell) were cultured in RPMI 1640 supplemented with 15% fetal bovine serum and 1% penicillin-streptomycin. All cells were grown at 37° C. with 5% C02. Adherent cells were passaged every 72 h using trypsin-EDTA (0.05%), or trypsin-EDTA (0.25%) for HepG2 cells. Where indicated, media were supplemented with estrogen (Sigma, Cat #E-060-1ML) dissolved in ethanol or an equal volume of vehicle. Human astrocytes (Sciencell, Cat #1800-5) were maintained in astrocyte media (Sciencell, Cat #1801) on poly-L-lysine (Sciencell Cat. #0413) coated culture dishes. Cells were passaged using trypsin/EDTA (Sciencell Cat #0183) and neutralization solution (Sciencell Cat #0113).

### Chromatin Immunoprecipitation

Foreskin fibroblasts were cross-linked with 1% formaldehyde for 10 min. Cross-linking was stopped with 2.5M glycine for 5 min. Nuclei were isolated by rotating crosslinked cells for 10 min at 4° C. in 5 ml lysis buffer 1 (50 mM HEPES pH 7.6, 140 mM NaCl, 1 mM EDTA, 10% glycerol, 0.5% NP-40, 0.25% Triton X-100) followed by pelleting, and a 10 min rotation in 5 ml lysis buffer 2 (200 mM NaCl, 1 mM EDTA, 0.5 mM EGTA, 10 mMTris, pH 8). Nuclei were pelleted, then swelled in lysis buffer 3 (10 mM Tris, pH 8, 1 mM EDTA, 0.5 mM EGTA, 100 mM

NaCl, 0.1% deoxycholic acid, 10% N-lauryl sarcosine) for 10 min, then sonicated on high setting (30 s on, 30 s off) for 15 min to shear chromatin to <500 nt with Bioruptor (Diagenode). After pelleting the insoluble fraction, the supernatant was pre-cleared with Protein A/G agarose beads (Thermo Fisher, Cat #78609) and anti-rabbit IgG (Sigma, Cat #I5006). 50 μg sheared chromatin was incubated in RIPA buffer (50 mMTris, pH8, 150 mM NaCl, 1% NP-40, 0.5% sodium deoxycholate, 0.1% SDS) with either 5 μg rabbit IgG (Sigma Cat #I5006), 5 μg mouse IgG (Santa Cruz, Cat #SC2025), or 5 μg of antibodies against NELFA (Santa Cruz, Cat #sc365004), H3K27ac (Abcam, Cat #ab4729) or H3K4me1 (Abcam, Cat #Ab8895) and recovered with Protein A/G beads. Beads were washed twice with low salt RIPA (150 mMNaCl) and twice in high salt RIPA (300 mM NaCl), then eluted in 100 μl 1% SDS plus 100 mM sodium bicarbonate. After cross-link reversal, DNA was purified with a QIAquick PCR Purification Kit (Qiagen) and quantified by qPCR or by sequencing. ChIP-seq libraries were prepared using the Ovation Ultralow Library system (NuGen). Libraries were sequenced on the HiSeq 2500 instrument (Illumina) and ~40 million 100-nt reads were generated per ChIP sample. Sequence pre-processing and alignment
were performed as described for PRO-seq.

### Co-Expression Analysis

Entrez gene IDs for APOE (348), APOC1 (341) and TOMM40 (10452) were submitted to COEXPRESSdb and expression data were plotted under default settings. Pearson correlation coefficients are reported.

### Expression Analysis

Total RNA was isolated using the RNeasy Mini-Kit (Qiagen) and 0.5 μg RNA converted to cDNA using TagmanRT reagents kit (ThermoFisher) with random hexamer priming. Gene expression was determined by SYBR green qPCR on an ABI 7900HT or BioRad CFX384 instrument using the delta-Ct method.

Oligonucleotides for qPCR (SEQ ID NOS: 3-14)

| Oligo | Sequence |
|---|---|
| APOE_F | CAGGTCACCCAGGAACTGAG |
| APOE_R | TCCAGTTCCGATTTGTAGG |
| AANCR_F | TCCTAACCTTAACCCAGAAGCA |
| AANCR_R | TGGGTTTTAATCACAGCTTCCT |
| Rloop_F | CCTCCCATCCCACTTCTGT |
| Rloop_R | AGCTACTTGGGAGGCTGAGG |
| GAPDH_F | CAATGACCCCTTCATTGACC |
| GAPDH_R | GACAAGCTTCCCGTTCTCAG |
| HPRT_F | TTATGGACAGGACTGAACGTCTTG |
| HPRT_R | CCAGCAGGTCAGCAAAGAATT |
| HSP70_F | AAGACTCTGGAGAGTTCTGAGCA |
| HSP70_R | CTCGGCTTTTATAAGTCGTCGT |

### Hypertonic Stress Treatment

HK-2 cells were cultured in DMEM-F12 media supplemented with 50 mM NaCl to a final 400 mOsm. To generate conditioned media HK-2 cells were maintained in isotonic media (300 mOsm) or hypertonic media (400 mOsm) for 6 h. After 6 h the conditioned hypertonic media was collected. The isotonic media was collected and 50 mM NaCl was added to yield hypertonic media. The hypertonic media and hypertonic conditioned media were confirmed to be 400 mOsm using a 6002 μOsm Osmometer (Precision systems). HK-2 cells were seeded in 96-well plates and maintained in hypertonic media or hypertonic conditioned media for 24 h. Apoptosis was monitored using RealTime-Glo™ AnnexinV Apoptosis and Necrosis Assay (Promega, Cat #JA1011) according to manufacturer specifications and quantified in a Cytation 5 (Agilent) plate reader.

RNA Abasic Site Detection and Quantitation with ARP

RNA samples were incubated in 2 mM ARP (N-(aminooxyacetyl)-N′-(D-Biotinoyl) hydrazine) (Thermo Fisher Scientific, Cat #A10550) in 20 mM Tris-HCl, 1 mM DTT, 1 mM EDTA, pH 8.0 at 37-C with agitation for 1 h. Formaldehyde was then added to 50 mM and incubated for 10 min at 37-C to quench ARP. RNA was precipitated in 0.3 M NaAc (pH 5.5) and 3 volumes of 100% EtOH, followed by 75% EtOH washes. The samples were analyzed on 1% formaldehyde agarose gel in 1×MOPS buffer (20 mM MOPS, 5 mM sodium acetate, 1 mM EDTA, pH 7.0). RNA was transferred to Hybond N+ nylon membrane by overnight capillary transfer in 10×SSC buffer (1.5 M NaCl, 150 mM sodium citrate, pH 7.0). Biotin signal on nylon membrane was detected using a streptavidin-based chemiluminescent method (Thermo Fisher Scientific, Cat #89880).

For site-specific quantification, ARP-labelled abasic RNA was recovered with M280 streptavidin beads. RNA was eluted from the beads using Trizol and converted to cDNA by reverse transcription using random hexamer priming. Enrichment of RNA containing abasic sites was quantified by qPCR.

### DNA-RNA Hybrid Immunoprecipitation (DRIP)

Immunoprecipitation procedure was adapted from previous studies (Skourti-Stathaki et al., 2011). 5×10⁶ primary fibroblasts were lysed in 600 μl cell lysis buffer (50 mM PIPES, pH 8.0, 100 mM KCl, 0.5% NP-40) and nuclei were collected by centrifugation. Pelleted nuclei were resuspended in 300 μl nuclear lysis buffer (25 mM Tris-HCl, pH 8.0, 1% SDS, 5 mM EDTA). Genomic DNA, along with R-loop, were then extracted by phenol:chloroform and ethanol precipitation. Purified DNA was resuspended in IP dilution buffer (16.7 mM Tris-HCl, pH 8.0, 1 mM EDTA, 0.01% SDS, 1% Triton-X100, 167 mMNaCl) and sonicated for 15 min in Bioruptor (Hi setting, 30 s on/30 s off) to fragments with average size of 500 nt. Three μg of S9.6 monoclonal

antibody (gift from Dr. Stephen H. Leppla at NIH) or non-specific mouse IgG (Santa Cruz, Cat #SC2025) was used for each immunoprecipitation. Input and precipitates were analyzed by quantitative PCR using primers or by sequencing. Sequencing libraries were prepared from input and DRIP DNA using Ovation Ultralow System (NuGen, Cat #0344) and sequenced on an HiSeq 2500 (Illumina). An average of 100 million 100 nt reads per sample were generated. Sequencing reads were pre-processed to remove the adapter sequences from the end of reads using the program fastx clipper from FASTX-Toolkit (Hannon Lab). Low-quality sequences at the ends of reads as represented by stretches of ‘#’ in the quality score string in the FASTQ file were also removed. Reads shorter than 35 nt after trimming were excluded from the analysis. Sequencing reads were then aligned to human reference hg18 using GSNAP (Version 2013-10-28) (20) using the following parameters: mismatches ≤[(read length+2)/12−2]; mapping score ≥20; soft-clipping on (-trimmismatch-score=−3). Reads with identical sequences were compressed into one unique sequence. BigWig tracks were computed using bedtools and converted to hg19 coordinates using CrossMap.

### Genetic Analysis

Genetic analysis of APOE expression was carried out using the eQTL calculator in GTEx (https://gtexportal.org/home/testyourown), we tested the allelic association of two SNPs in AANCR, rs449647 and rs405509 with APOE expression in the brain, colon, liver, and testis, each SNP and expression pair tested individually. The nominal P-values (<0.05) are shown in. Allelic association of rs449647 with APOE plasma level is obtained from the publication (21). Association of rs449647 and rs405509 with Alzheimer's disease was analyzed using the GWAS Catalog and the NIAGenomics of Alzheimer's disease using the two SNP IDs individually in the search field, https://www.ebi.ac.uk/gwas/ and https://www.niagads.org/genomics/home.jsp.

iPSC Hepatocyte Differentiation

Hepatic differentiation of human pluripotent stem cells was performed following a three-step protocol (22). First, iPSCs at 60-70% confluence were treated for 3 days with 100 ng/ml Activin A (R+D Systems, Cat #338-AC) and 100 ng/ml bFGF (R&D Systems, Cat #3718-FB) in the presence of increasing levels of FBS (0% on day 1, 0.2% on day 2, and 2% on day 3) to generate definitive endoderm. Confluent definitive endoderm cells are then passed 1:3 in presence of Rock Inhibitor (Tocris, Cat #1254) on growth factors reduced Matrigel (BD Biosciences, Cat #354277)

and cultured for 8 days in differentiation medium: DMEM F12, 10% KOSR (Sigma, Cat #10828010), with 1% NEAA (Thermo Fisher, Cat #11140050), 1% glutamine, 100 ng/ml of HGF (Peprotech, Cat #100-39H) and 1% DMSO(Sigma Aldrich, Cat #D2650), to promote hepatic specification. Finally, the hepatoblasts were matured in DMSO-free differentiation medium with 10-7M of dexamethasone (Sigma Aldrich, Cat #D8893) for 3 days. Hepatocytes were then maintained for up to 1 week in hepatocyte culture medium: L15 medium (Cat #11415064), 8.4% FCS with 1% glutamine, 10% tryptose phosphate (Life Technologies, Cat #18050039) containing 1 μM insulin (Sigma, Cat #I3536), 10 μMhydrocortisone (Sigma, Cat #H0888) and 0.1 μM of dexamethasone.

### MPG Electrophoretic Mobility Shift Assay

RNA/DNA hybrid substrates containing a single N6-methyl-adenosine within the RNA strand was incubated with increasing concentration of human recombinant MPG. MPG protein was purified as described previously (23). Binding incubations were performed on ice for 15 min in buffer containing 20 mM Tris-HCl, pH 8.8, 10 mM (NH₄)₂SO₄, 10 mM KCl, 2 mM MgSO₄, 0.1% Triton X-100 and a range of 0-800 nM of MPG. The binding mixtures were immediately subjected to non-denaturing 6% polyacrylamide gel (acrylamide:bisacrylamide, 37.5:1) electrophoresis. To maintain the integrity of bound complexes during electrophoresis, the gel was run at 4° C. The fraction bound relative to the total signal was then determined and plotted using Kaleidagraph v.4.1. The data from ≥3 binding experiments were fitted to a modified Hill binding equation, where the fraction of substrate-bound is related to the Kd as described (24):

$$f = ( \frac{{f}_{max} * {[ E ]}^{n}}{{[ E ]}^{n} + {K}_{d}^{n}} ) + {f}_{min}$$

where fₘₐₓ and fₘᵢₙ are normalization factors that represent the fraction of substrate bound at the highest and lowest asymptotes of the titration, [E] is the total enzyme concentration, and n is the Hill coefficient. The Hill coefficient measures the cooperativity of binding. Kd values were estimated from the fitted data.

### MPG Enzymatic Activity Assay

RNA/DNA hybrid substrates containing a single N6-methyl-adenosine were formed by heating to 90° C. for 1 min and slowly cooling to 4° C. in a buffer containing 30 mM Tris, pH 7.5, and 100 mM potassium acetate. The strand containing the methylated adenosine was 5′-end labeled with fluorescein (FAM). Annealed substrates (200 nM) were incubated with (+) or without (−) 20 units of MPG in a reaction buffer containing 20 mM Tris-HCl, pH 8.8, 10 mM (NH₄)₂SO₄, 10 mMKCl, 2 mM MgSO₄, 0.1% Triton X-100 for 60 min at 37° C. Substrates were then treated with 1 μM APE1 (Wilson lab) for an additional 2 min at 37-C. Enzymes were subsequently inactivated by a 5-min incubation at 75° C. Samples were then denatured with formamide (1:1 vol/vol) and a 2-min incubation at 95° C. before loading onto a 7 M urea 15% denaturing polyacrylamide gel.

Substrates used for MPG assay (SEQ ID NOS: 15-22)

| Name | sequence (5′→3′) |
|---|---|
| 5′FAM-RNA-6mrA- | /56-FAM/ArArArUrArCrCArCrUrArCrCrUrUtUrArU/iN6Me- |
| position 19 | rA/rGrUrUrUrCrUrUrUrArCrUrUrArUrUrCrArArU |
| Reverse DNA (6-mrA | ATTGAATAAGTAAAGAAACTATAAAGGTAGTGGTATTT |
| complement) | |
| 5′FAM-DNA-6mdA- | /56-FAM/ATTGAATAAGTAAAGAAACT/iN6Me- |
| position 21 | A/TAAAGGTAGTGGTATTT |
| Reverse RNA (6-medA | rArArArUrArCrCrArCrUrArCrCrUrUrUrArUrArGrUfUfUfCrUrUrUrArCr |
| complement) | UrUrArUrUrCrArArU |
| Reverse RNA (6-merA | rArUrUrGrArArUrArArGrUrArArArGrArArArCrUrArUrArArArGrGrUrA |
| complement) | rGrUrGrGrUrArUrUrU |
| Reverse DNA (6-mdA | AAATACCACTACCTTTATAGTTTCTTTACTTATTCAAT |
| complement) | |
| 5′FAM-UND-RNA | /56- |
| (NO 6mrA) | FAM/rArArArUrArCrCrArCrUrArCrCrUrUrUrArUrArGrUrUrUrCrUrUrU |
| | rArCrUrUrArUrUrCrArArU |
| 5′FAM-UND-DNA | /56-FAM/ATTGAATAAGTAAAGAAACTATAAAGGTAGTGGTATTT |
| (NO 6mdA) | |

### MPG RIP-seq

For MPG RIP-seq, following isolation of RNA, sequencing libraries were prepared using TruSeq Stranded Total RNA Library Prep Kit (Illumina). Sequencing was performed on Illumina HiSeq 2500. Sequencing adapters and low-quality read ends were trimmed using the FASTXToolkit and reads shorter than 35 nt were discarded. Reads were mapped to GRCh37 (hg19) using GSNAP (v20190912) with parameters ‘-B 4-N 1-M 1-n 10-Q-max-mismatches=5’. Reads with identical sequences were collapsed to a single read. Input read depth was smoothed using a 1000 nt window, and MPG peaks were called by computing a ratio of RIP/smoothed input coverage at each base. Regions larger than 50 bases with ratio ≥5 (represented

by three or more reads) were considered RIP peaks.

### m6A RIP-seq

For m6A RIP-seq, following isolation of RNA, sequencing libraries were prepared using TruSeq Stranded Total RNA Library Prep Kit (Illumina). Sequencing was performed on Illumina MiSeq. Sequencing adapters and low-quality read ends were trimmed using the FASTX-Toolkit and reads shorter than 35 nt were discarded. Reads were mapped to GRCh37 (hg19) using GSNAP (v20190912) with parameters ‘-B 4-N 1-M 1-n 10-Q (note: no maxMismatches=5 flag)’. Reads with identical sequences were collapsed to a single read, then BigWig files were generated and plotted as a heatmap using deepTools 3.5.1. RNA-sequencing

From cells treated with siRNA against MPG or non-target siRNA, sequencing libraries were prepared using TruSeq Stranded TotalRNALibrary Prep Kit (Illumina). Sequencing was performed on Illumina HiSeq 2500 and >150 million 100-nt reads were generated from each sample. Low quality bases were trimmed from the 3′ end of reads and 3′ adapter was trimmed using FASTQ/A Clipper with default settings (Hannon lab). Reads shorter than 35 nt were excluded from analysis. Sequencing reads were aligned to human reference (hg19) using GSNAP (v20190912) (Wu and Nacu, 2010) using the following parameters: mismatches % ([(read length+2)/12-2); mapping scoreR20; soft-clipping on (-trim-mismatch-score=3). Precision run-on sequencing (PRO-seq)

PRO-seq libraries were prepared as described previously (25,26). Fibroblast nuclei (5×106) were added to 2×nuclear run-on (NRO) reaction mixture (final concentrations: 10 mM Tris-HCl pH 8.0, 300 mM KCl, 1% sarkosyl, 5 mM MgCl₂, 1 mM DTT, 0.03 mM each of biotin-11-A/C/G/UTP (Perkin-Elmer, Cat #NEL544001EA, Cat #NEL543001EA, Cat #NEL542001EA and Cat #NEL545001EA), 0.8 u/μl RNase inhibitor) and incubated for 3 min at 37° C. Nascent RNA was extracted by phenol (Trizol LS)/chloroform and then fragmented by base hydrolysis in 0.2 N NaOH on ice for 15 min. The reaction was neutralized by adding 0.7×volume of 1 M Tris-HCl pH 6.8. The fragmented nascent RNA was purified using 30 μl of Streptavidin M-280 magnetic beads (Thermo Fisher Scientific, Cat #11206D) and ligated with 3′ RNA adapter (5′p-GAUCGUCGGACUGUAGAACUCUG AAC-/3InvdT/(SEQ ID NO: 23)). Biotin-labeled products were recovered by streptavidin beads. The RNA products were successively treated with 5′ pyrophosphohydrolase (NEB, Cat #M0356) and polynucleotide kinase (NEB, Cat #M0201) to repair the 5′ end. RNA was ligated to the 5′ RNA adapter (5′-CCUUGGCACCCGAGAAUUCCA-3′ (SEQ ID NO: 24)). The products were further purified by the streptavidin beads. RNA was reverse transcribed using RT primer (5′-AATGATACGGCGACCACCGAGATCTACACGTTCAGAGTTCTACAGTCCGA-3′ (SEQ ID NO: 25)). The product was PCR amplified, resulting amplicons that are between 150 and 250 bp (insert >70 bp) were purified using the BluePippin (Sage Science) agarose gel electrophoresis, and then sequenced on the HiSeq 2500 instrument (Illumina) to a depth of >150 million reads per sample. PRO-seq data were aligned to the human genome using GSNAP, results corresponding to GRCh37 (hg19) are shown (20). BAM files were generated and normalized to reads per million

mapped reads (RPM). For comparison between conditions, RPM normalized signal was plotted for AANCR-APOE locus.

The pausing index (PI) was calculated as the ratio of the read density for the 50 nt interval in AANCR with the greatest number of reads and compared to the read density over the rest of the noncoding transcript. A region with a PI>3 was considered as having paused polymerase.

### RNA Immunoprecipitation

Primary human fibroblasts (5×10⁶ cells per experiment) were treated with lysis buffer, (10 mM Tris-HCl pH 7.4, 10 mM NaCl, 0.5% NP-40, 1 mM DTT, 200 units/ml RNase OUT and EDTA-free protease inhibitor cocktail), the lysate mixed with a freshly made preparation of protein A/G magnetic agarose beads bound to either anti-MPG antibody, anti m6A antibody or anti-METTL3 antibody. Protein-RNA complexes in the cell lysates were allowed to bind to their respective antibody-bead preparation, followed by treating the immunoprecipitants with proteinase K, RNA extraction and DNase treatment of the extracted RNA. Samples were converted to cDNA using random hexamers and enrichment was assessed by qPCR.

### Elongating RNA Pol II Pausing in Noncoding Transcripts

To identify noncoding transcripts with paused RNA Pol II, data from the DBKERO TSS-seq database (Release 1.2.7), PRO-cap and corresponding PRO-seq (26,27) were used. A noncoding transcript is identified as one with TSS and nascent RNA (from PRO-seq) and it is located at least 200 bases from an annotated coding gene.

For each noncoding transcript, we assessed for evidence of paused RNA Pol II. Each transcript was divided into 50-nucleotide windows and the average PRO-seq read counts was determined. Paused RNA Pol II were identified when the average count in a 50-nucleotide window is at least three times greater than the average read count in the remaining transcript. For the 50-nucleotide windows that are at least 500 bases from TSS, we considered those to be RNA Pol II that paused in gene bodies (during elongation). The gene body pauses that overlap R-loops (annotated consensus locations from RLBase (28) (version 1.0.1)) and MPG binding sites (MPG RIP-seq) were then identified. The regions in B cells, fibroblasts and renal proximal tubule cells where RNA Pol II pause in gene bodies, and colocalize with R-loops and MPG binding are reported.

siRNA Knockdown

Primary fibroblasts were seeded at 2×10⁵ per well in 6-well dishes. Cells were transfected with siRNA using Lipofectamine RNAiMax to a final concentration of 12 nM on day 0 and day 3. Cells were harvested for expression or protein analysis 3 and/or 7 days post-transfection. Catalog

numbers for siRNA targeting MPG, METTL3 and control siRNA are listed in Supplementary methods.

### S9.6 Dot Blot

S9.6 dot blot to assess genome-wide R-loop abundance was carried out as before (29). Briefly, genomic DNA containing R-loops was incubated with 1 μl of RNase H1 or mock digestion in 1×RNase H reaction buffer (10 mM Tris-HCl pH 8.0, 50 mM NaCl, 10 mM MgCl2, 10 mM DTT) at 37-C for 30 min. DNA was phenol extracted, ethanol precipitated, and reconstituted in 10 μl TE buffer. 5 μl DNA solution was loaded onto Hybond N+ nylon membranes (GE Life Sciences, Cat #RPN203B) presoaked with PBS, and crosslinked in UV Stratalinker 2400 (Stratagene) at the ‘Auto Crosslink’ setting (1200 PJoulesX100). The membrane was blocked in 5% milk in PBS-0.1% Tween-20 for one hour and incubated with 1:1000 S9.6 antibody overnight at 4° C. to detect RNA/DNA hybrids. A duplicate blot was incubated with anti-dsDNA antibody (Abcam, Cat #ab27156) as a loading control. Signals were then detected by horse-radish peroxidase (HRP)-conjugated secondary antibody and enhanced chemiluminescence. The S9.6 signals normalized to dsDNA signals were determined.

### Secreted APOE Detection

To measure secreted APOE, cell culture media was concentrated 10-fold using an Amicon ultra centrifugal device (Sigma). Concentrated media (500 μl) was mixed with an equal volume of 2×RIPA buffer with protease inhibitor (Sigma, Cat #11836170001) and PMSF (Sigma, Cat #P7626) and rotated overnight with 10 μg APOE antibody (Sigma, Cat #AB947) or IgG (Sigma, Cat #I5006). Antibody-protein complexes were recovered with protein A/G beads (Fisher, Cat #PI78609) after 2 h of rotation at 4-C and three washes in 1×RIPA. Bound protein was released in 30 μl sample loading buffer, and western blot was performed by standard procedure. APOE was detected with 1:1000 primary antibody (Abcam) and 1:5000 secondary antibody. Ponceau (Sigma, Cat #P3504) staining of the western membrane was used as the loading control.

### White Blood Cell Isolation

WBC were obtained from a subject who received clinical evaluations at the National Institutes of Health (NIH) in Bethesda, MD under IRB-approved protocol 00-N-0043 ‘Clinical and Molecular Manifestations of Inherited Neurological Disorders.’ Written informed consent was received from the participant before inclusion in the study. Venous blood was collected in a 10 ml lavender top K2EDTA tube. 30 ml of RBC lysis solution (Qiagen) was added to 10 mL of whole blood and mixed by inverting 10 times. The sample was incubated for 5 min at room temperature. WBCs were pelleted by centrifugation for 2 min at 2000×g.

### Statistics

This study included various statistical approaches which are detailed in the appropriate subsections in the methods. Publicly available sequencing data that support the conclusions of the study are listed in Supplementary methods. The deep sequencing data reported in this paper have been deposited in the NCBI sequence read archive (PRJNA801792) and the NCBI database of Genotypes and Phenotypes archive (Phs001322.v2.p1). The number of biologically independent

experiments, sample size, statistical tests, and P-values are indicated in the main text or the figure legends. The significance level was set at P<0.05 or less.

### Results

### Methylpurine Glycosylase Knockdown Induces APOE Expression

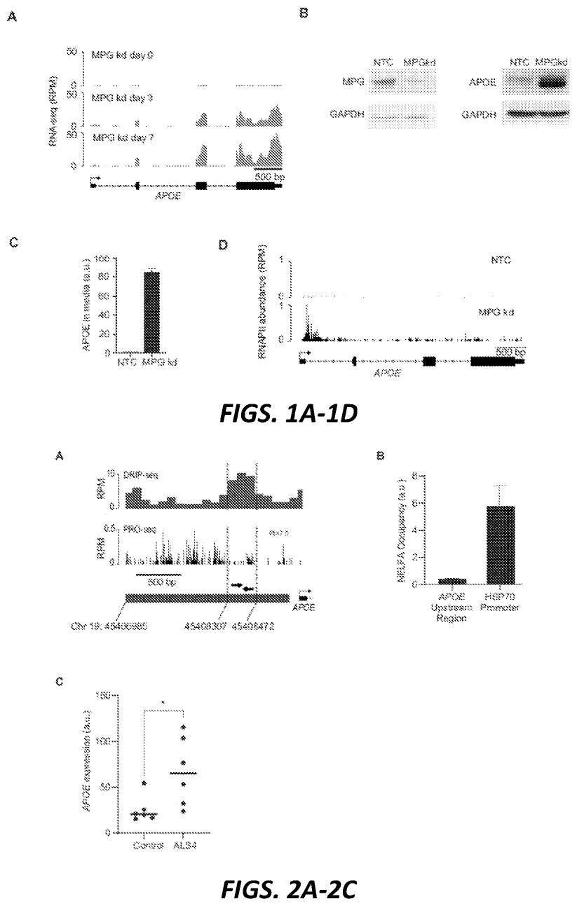

To study RNA abasic sites, in skin fibroblasts, MPG was knocked down by RNA interference and sequencing was carried out RNA which showed that the most highly induced gene is APOE. APOE is not expressed or at a very low level in skin fibroblasts, but when MPG is knocked down, APOE gene expression is highly induced (FIG. 1A, >50-fold). The immunoblot shows that APOE protein expression is also upregulated significantly (FIG. 1B, P<<0.001, >20-fold). The resultant APOE is then secreted, as APOE protein level is significantly higher (FIG. 1C, P<0.001, >50-fold) in the cell culture media for the fibroblasts whose MPG is knocked down. MPG was also knocked down in lung epithelial cells and it was found that APOE protein expression is also induced in these cells. Since APOE is not expressed or at a very low level in skin fibroblasts, the increase in APOE expression is most likely a result of increased transcription. To measure RNA polymerase II (RNA Pol II) transcription, Precision nuclear Run-On sequencing (PRO-seq) was carried out which identifies nascent RNA with actively transcribing RNA Pol II. The PRO-seq results show that indeed upon MPG knockdown, there is an increase in the abundance of RNA Pol II in the APOE promoter and active transcription of APOE (FIG. 1D). Thus, MPG knockdown induces transcription and expression of APOE.

### R-Loops Form Upstream of APOE and Regulate its Expression

A previous study showed that MPG forms abasic sites in the RNA of RNA/DNA hybrids but not in double stranded RNA (18). Here, it was asked whether MPG affects APOE expression through binding to R-loops. Using the S9.6 antibody that specifically recognizes R-loops (30-32),

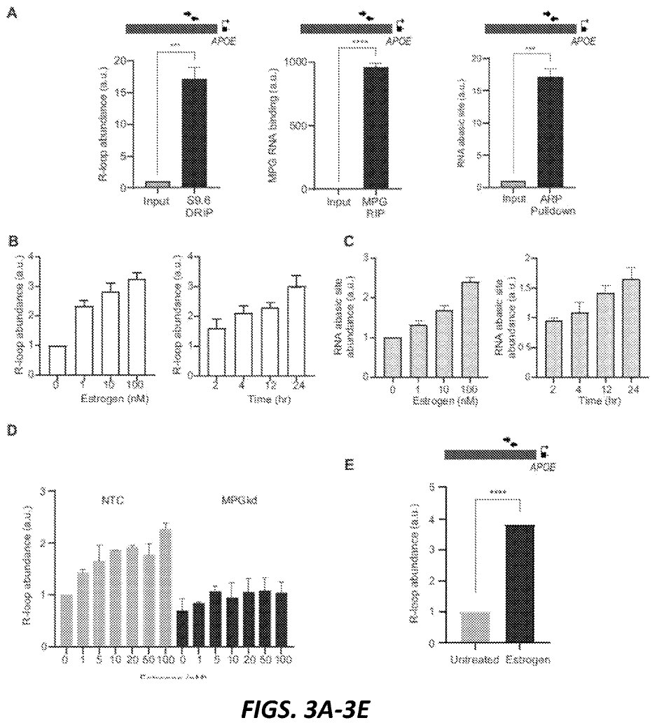

DNA-RNA immunoprecipitation, DRIP, was carried, followed by sequencing to enrich and map R-loops. Since APOE is not transcribed or at a very low level in fibroblasts, there was no RNA to form R-loops in APOE, rather R-loops upstream of APOE were detected (FIG. 2A, top panel), in an intergenic region. This upstream region is transcribed by RNA Pol II until the polymerases pause (pausing index=7.5), just 5′ to the R-loops (FIG. 2A, bottom panel). To assess if the R-loops or negative elongation factor (NELF) protein complex pauses the RNA Pol II transcription
(33), an immunoprecipitation was carried against NELFA. In NELFA-IP followed by PCR, NELFA binding to the region upstream of APOE was not detected, whereas NELFA binding was readily detected at the known NELF-mediated RNA Pol II pause site in the HSP70 promoter (FIG. 2B). This suggests that in the region upstream of APOE, RNA Pol II transcription is paused by R-loops.

Do the R-loops affect APOE expression?APOE expression was examined in ALS4 patient

cells. ALS4 is caused by heterozygous senataxin mutations that lead to a hyperactive senataxin (RNA/DNA helicase) that reduces R-loops without affecting transcription initiation (7,8). APOE expression in fibroblasts from ALS4 patients and their family controls were compared. It was found that on average, ALS4 patients with fewer R-loops (8) have higher APOE expression (FIG. 2C). While APOE is not expressed in most of the fibroblasts from controls, it is expressed in many of the fibroblasts from ALS4 patients. Thus, cells with fewer stable R-loops have higher APOE expression. Like MPG expression, R-loop abundance is negatively correlated with APOE expression.

### RNA Abasic Sites Form in the R-Loops and Pause RNA Pol II Transcription Upstream of APOE

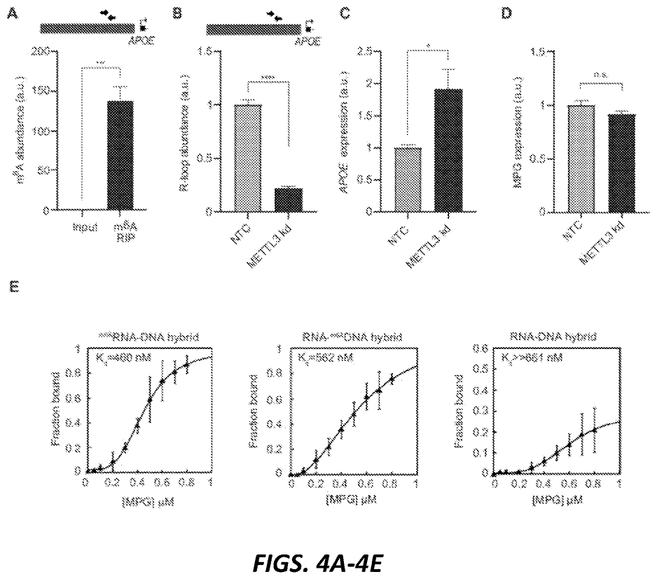

Next, it was asked if the MPG binds to the R-loops near APOE and if so, are RNA abasic sites generated. MPG RNA-IP and ARP RNA-pulldown were carried out. The presence of R-loops upstream of APOE as shown in FIG. 2A (FIG. 3A, left panel) was confirmed by S9.6 DRIP-PCR. MPG RNA-IP then shows that MPG binds to the RNA in those R-loops upstream of APOE (FIG. 3A, middle panel), and ARP RNA-pulldown (FIG. 3A, right panel) shows abasic sites in the RNA. Together, the results show that MPG binds to the R-loops upstream of APOE and forms RNA abasic sites.

To further examine the relationship between R-loops and RNA abasic sites, R-loops were induced with estrogen (34). In primary fibroblasts, estrogen significantly (P<0.0001) increases R-loops (FIG. 3B) and RNA abasic sites (FIG. 3C) in a dose and time dependent manner genome-wide. Since RNA abasic sites form on R-loops and given that estrogen increases R-loops, it is not surprising that the abundance of RNA abasic sites also increases following estrogen. To assess if there is a reciprocal relationship between R-loops and RNA abasic sites, MPG was knocked down and those fibroblasts were treated with estrogen. Results show that when MPG is knocked down, R-loops do not accumulate in response to estrogen (FIG. 3D), indicating that the stability of R-loops may depend on RNA abasic sites.

The estrogen treatment increases R-loops genome-wide, including upstream of APOE (FIG. 3E). The effect of the stabilized R-loops on transcription upstream of APOE was assessed by performing PRO-seq in fibroblasts at different timepoints following estrogen treatment. The estrogen induced R-loops led to increased pausing of RNA Pol II in the intergenic region, and a time-dependent increase in RNAPol II pausing was observed (pausing index increased from 4.8 in resting cells to 11.8 after 6 h in estrogen).

Together, these results show that upstream of APOE, nascent RNA forms R-loops, then MPG generates RNA abasic sites which likely stabilizes the R-loops that in turn pause transcription of the intergenic RNA.

### m6A is Likely a Precursor to RNA Abasic Site in R-Loops

It was then asked what attracts MPG to the RNA of the Rloops upstream of APOE. Since MPG is a methylpurine glycosylase, the focus was on methylated purines. In Modomics (3), there are 50 different types of methylated purines in RNA. Among them, m6A is the most abundant (35-38). Given their abundance and based on findings from previous studies that identified m6A in R-loops (39-41), the focus was on m6A to ask if they are present in the RNA in the R-loops upstream of APOE and if so, whether they are a target of MPG.

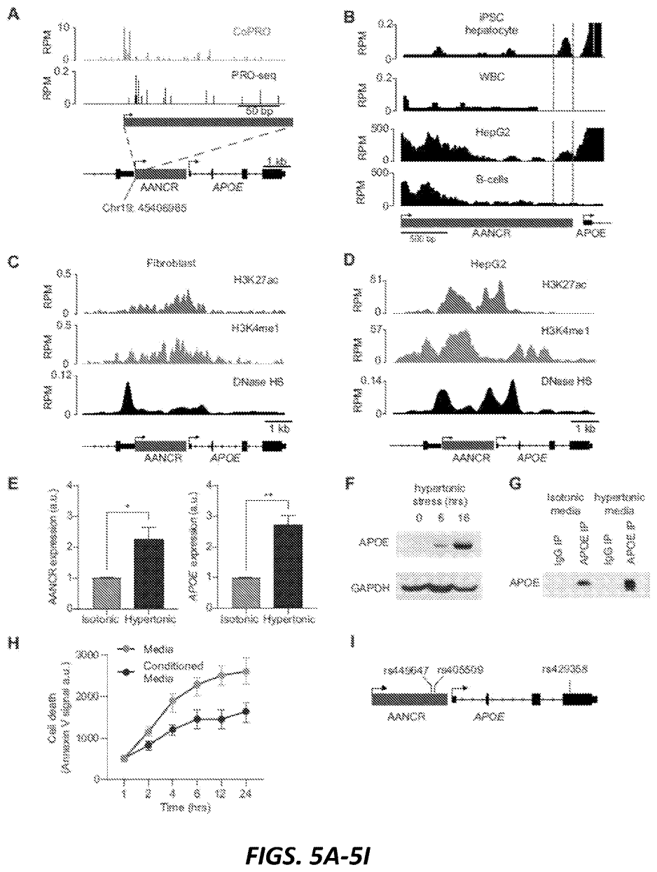

In that RNA, the DRACH (D=A/G/U, R=A/G, H=A/C/U) motif (42,43) where methyltransferases such as METTL3 and METTL14 methylate adenosines. m6A RNA-IP was then carried out which identified m6As in the RNA upstream of APOE (FIG. 4A) and it coincides with where MPG binds (FIG. 3A, middle panel). The colocalization data suggest that m6As in the R-loops may be the substrate of MPG and therefore the precursor to RNA abasic sites.

If m6A is a substrate of MPG, then the METTL3/METTL14 complex that methylates adenosine to form m6A (44,45) should also regulate APOE expression. METTL3 was knocked down and the abundance of the R-loops upstream of APOE and APOE expression were measured. In cells whose METTL3 was knocked down, significantly (P<0.001) fewer R-loops (FIG. 4B) and significantly (P<0.05) higher APOE expression (FIG. 4C) without affecting the expression of MPG (FIG. 4D) was found. Hence, METTL3 knockdown phenocopies MPG knockdown, and METTL3 likely acts upstream of MPG in the regulation of APOE.

It was further assessed if m6A is a substrate of MPG by biochemical assays. In a previous study (18), MPG was knocked down and RNA abasic sites were measured by mass spectrometry which showed that RNA abasic sites decreased significantly. It was found that MPG excised the hypoxanthine in RNA/DNA hybrids when inosine was in the RNA strand while MPG activity was minimal in the RNA/DNA hybrid without inosine (18). The cleavage site was mapped with AP-endonuclease 1 which incised at the RNA abasic sites of RNA/DNA hybrid but not in double-stranded RNA (18).

Those findings are extended herein and assessed for binding and cleavage of the same RNA/DNA hybrid as in the previous study (18) except the RNA strand contains an m6A and not an inosine. In electromobility shift assays, it was found that MPG has a stronger affinity for the hybrid with m6A RNA than its affinity for the hybrid with m6A DNA, or unmodified RNA/DNA hybrid (FIG. 4E). It was then assessed if MPG cleaves the m6A by incubating the RNA/DNA hybrid with MPG followed by AP endonuclease 1 (18) which incises the sugar-phosphate backbone at abasic sites. The results showed incision of the RNA/DNA hybrid withm6A in the RNA, but there was no incision of the hybrid with m6A in the DNA strand nor the RNA/DNA hybrid with no modification. This suggests that m6A is removed by MPG to form an RNA abasic site. Taken together the genetic and biochemical findings point to m6A as a likely precursor of RNA abasic site in the R-loop upstream of APOE.

### AANCR, a Non-Coding RNA Upstream of APOE

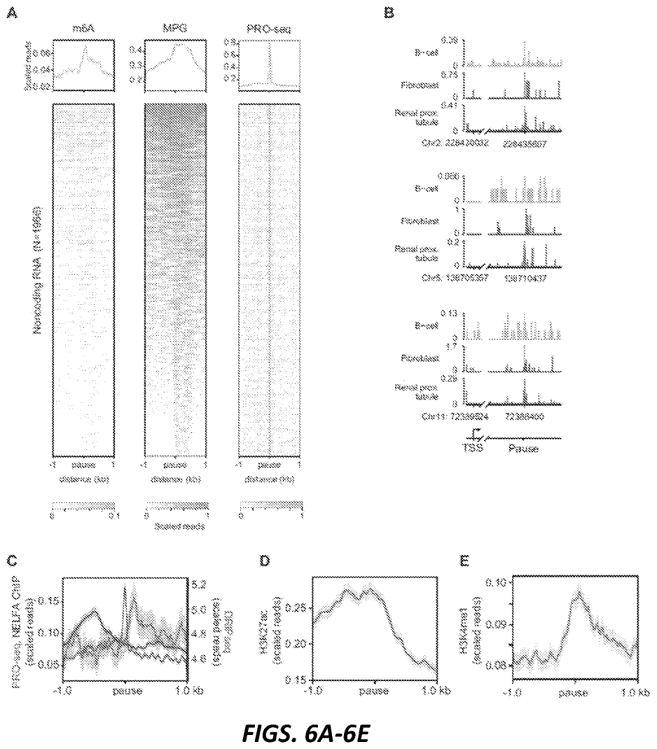

Thus far, it has been discussed that the R-loops that form upstream of APOE, without characterizing the RNA. The PRO-seq data show that the intergenic region is more actively transcribed than the upstream gene, Translocase of Outer Mitochondrial Membrane 40 (TOMM40), and the downstream gene, APOE. There are more RNA Pol IIs in the intergenic region than in TOMM40 and APOE; thus, the RNA is an independent transcript and not part of TOMM40 or APOE. With data from Coordinated Precision Run-on sequencing (CoPRO; FIG. 5A top panel) (46) and PRO-cap (47,48) that map the 5′cap of RNA, a capped RNA corresponding to the intergenic transcript was identified. It was then confirmed that the capped RNA coincides with the nascent RNA identified by PRO-seq (FIG. 5A). A polyadenylation motif was searched for and not found in this RNA.

Next, it was asked if this capped RNA is noncoding. Sequence analysis by BLASTX (49) and PFAM (50) did not identify similar proteins. Additionally, the sequence was analyzed by several algorithms that test for coding potentials (CPAT, CPC, CNIT (51-53)). These analyses determined that this is a noncoding RNA (Table 2). Together, the results show that upstream of APOE is a capped noncoding RNA, which we have named APOE-associated noncoding RNA (AANCR).

TABLE 2
The RNA upstream of APOE is a noncoding RNA

| Method | Results |
|---|---|
| BLASTX | Only 3 sequences showed > 80% identity, two are |
| | hypothetical proteins, one is a low-quantity protein. |
| PFAM | No significant hits by PFAM-A HMM and GA cutoffs |
| CPAT | Fickett score = 0, hexamer score = 0, coding |
| | potential = 0.003; classified as noncoding |
| CNIT | Score = 0.31, classified as noncoding |
| CPC | Coding probability = 0.04, classified as noncoding |

The analysis thus far focused on primary skin fibroblasts that do not express APOE. Next, the analysis was extended by performing RNA-seq of other cell types, including liver cells that express APOE and white blood cells that do not express APOE, to assess if the length of AANCR correlates with APOE expression. FIG. 5B shows in iPSC-derived hepatocytes (22), AANCR is transcribed as a full-length transcript, but in white blood cells (from the same individual from which the hepatocytes were derived), AANCR is only partially transcribed. To confirm that in cells with full length AANCR, APOE is expressed but in cells with partial transcription of AANCR, APOE is not expressed, BRU-seq was used which maps nascent RNA by bromouridine tagging. BRU-seq data (54) was used in the ENCODE data portal (55). Those data also show full-length AANCR in liver cells that express APOE, but the partially transcribed AANCR is found in cultured B-cells where APOE is not expressed (FIG. 5B). In the liver cells where AANCR is full-length, no R-loops, m6As, or RNA abasic sites were found confirming that without stable R-loops, AANCR is transcribed as a full-length RNA that enhances APOE expression.

In summary, a noncoding RNA, AANCR, was discovered upstream of APOE and that AANCR is regulated by R-loops with RNA base modifications. In liver cells, AANCR is full-length and APOE is expressed; in contrast, in fibroblasts and B-cells, R-loops pause RNA Pol II transcription, so AANCR is only partially transcribed, and APOE is not expressed.

AANCR is an enhancer RNA Next, it was assessed if this noncoding RNA is an enhancer of APOE. The ENCODE registry of candidate cis-regulatory elements (56) had identified the region upstream of APOE as a potential enhancer based on DNase hypersensitivity and histone modification. GeneHancer also annotated AANCR as an elite enhancer based on the criteria that the region has features of enhancer chromatin and a strong enhancer-gene association (57). In primary fibroblasts, chromatin immunoprecipitation was performed followed by sequencing (ChIP-seq) to assess if the AANCR region has features consistent with an enhancer. In the AANCR region, H3K27ac and H3K4me1 marks were found, and DNase hypersensitive chromatin that characterize enhancers (58,59) (FIG. 5C). In HepG2 cells where APOE is expressed, as expected this region is also marked with open chromatin and enhancer marks, H3K27ac and H3K4me1 (FIG. 5D). Thus, AANCR is an enhancer RNA, and the region that compasses AANCR and APOE is poised for transcription even in cells where APOE is not expressed.

It was then asked whether AANCR physically interacts with APOE. Hi-C data was utilized to map chromatin folding at high resolution (60). The results showed that AANCR is in a ~18 kb topology-associated domain with APOE and the downstream gene apolipoprotein C-I (APOC1). In contrast, TOMM40 which is 12 kb upstream of AANCR is outside of that topology-associated domain. Following MPG knockdown, like APOE, APOC1 is significantly induced (P<0.001, 17-fold), whereas TOMM40 is only slightly increased (1.4-fold). Using the CoXpresDB (61) which allows users to query co-expression in >25 000 datasets from microarray and RNA-seq experiments in the public data bank (62), it was found that the expression levels of APOE and APOC1 are highly correlated, while APOE and TOMM40 expression are much less correlated. Thus, the finding that AANCR is an enhancer of APOE and APOC1 is a general phenomenon, beyond the cell types that were examined in this study.

Together, a non-coding RNA, AANCR, which enhances APOE and APOC1 expression were identified. In cells where base modifications stabilize R-loops, AANCR is only partially transcribed and cannot promote APOE and APOC1 expression, while in cells such as those in the liver, AANCR is a full-length enhancer RNA that induces transcription and expression of APOE and APOC1.

### AANCR and APOE Expression Levels are Stress-Responsive

Data such as DNase hypersensitivity from the ENCODE consortium and in the inventor's labs show that the APOE region is poised for expression in many cell types. This is somewhat surprising since APOE is known to be expressed only in several cell types, such as hepatocytes, macrophages, and astrocytes. It was posited that APOE is poised to be transcribed in response to stress. To test this, osmotic stress was performed. The kidney is subjected to hypertonic stress from the administration of medications and disease states such as hyperglycemia, which can lead to injury of the renal proximal tubule cells. Renal proximal tubule cells were treated with hypertonic salt and AANCR and APOE expression were measured. The results showed that in response to osmotic stress, renal proximal tubule cells express full-length AANCR which induces APOE expression (FIG. 5E). The resultant APOE transcripts are translated into APOE proteins and secreted into the culture media (FIGS. 5F and G). The cells were then treated in hypertonic conditions and media collected with the secreted APOE to culture cells. It was found that in the conditioned media which has more APOE proteins, that cells are more resistant to apoptosis despite the hypertonic stress (FIG. 5H). Together, these results show that AANCR acts as an enhancer to facilitate APOE expression in response to cellular stress.

### AANCR is a Modifier of Alzheimer's Disease

Since AANCR regulates APOE expression, it was questioned if it may affect susceptibility to Alzheimer's disease given that the F4 allelic form of APOE is a major risk factor for Alzheimer's disease (63). As a complex human disease, multiple factors contribute to Alzheimer's disease. Besides the F4 variant, other genetic factors have been identified, including those upstream of APOE where AANCR is encoded (FIG. 5I). Studies have reported an allelic association of rs449647 (also referred to as −491) with Alzheimer's (64-66). The GWAS catalog also shows multiple studies that have identified significant allelic associations for SNPs in the AANCR region including, rs449647, with Alzheimer's Disease (Supplementary Table S1). A study of 42 034 patients and 272 244 controls in the United Kingdom found a significant allelic association (P<10-58) of the SNP rs449647 with Alzheimer's disease (67). To assess how the sequence variants in AANCR affect susceptibility to dementia, it was asked if the genetic variants affect the function of AANCR and therefore APOE expression. Using data from the GTEx consortium (68), despite the small sample size of a few hundred samples, it was found several SNPs in the AANCR region that have significant allelic association with APOE expression, including in the hippocampus (Supplementary Table S2). The APOE signal from the hippocampus is most likely contributed by astrocytes where AANCR is expressed, and its expression is highly correlated with APOE expression (r=0.66; P<0.002 Spearman) (19).

TABLE S1
Examples of SNPs in AANCR region that show allelic
association with Alzheimer's Disease.

| SNP in | P-value* | |
|---|---|---|
| AANCR | (Alzheimer's Disease) | Study |
| rs449647 | 2 × 10⁻⁵⁸ | 42.034 British ancestry individuals with parental |
| | | history of Alzheimer's disease, at least 272,244 |
| | | individuals with no parental history of Alzheimer's |
| | | disease (Marioni et al., 2018) |
| | 8.7 × 10⁻¹⁸ | Transethnic LAOD (Jun et al., 2017) |
| | 2.6 × 10⁻³¹ | Meta-analysis of 74,046 individuals (Lambert et |
| | | al., 2013) |
| rs405509 | 1 × 10⁻⁸ | 952 European ancestry male cases, 1,789 European |
| | (males) | ancestry female cases, 6.337 European ancestry |
| | 4 × 10⁻¹⁴ | male controls, 8,402 European ancestry female |
| | (females) | controls (Nazarian et al., 2019) |
| | 3 × 10⁻⁴⁴ | ADGC GWAS Stage 1 (Naj et al.. 2011) |
| | 6.1 × 10⁻¹² | ADGC African American (Reitz et al.. 2013) |

*Obtained from GWAS catalog and the National Institute of Aging Genomics of Alzheimer's Disease database.

TABLE S2
Association results for SNPs in AANCR with
APOE expression level as phenotype.

| SNP in | Expression | Nominal | | |
|---|---|---|---|---|
| AANCR | phenotype | P-Value* | Tissue | Sample |
| rs449647 | APOE | 0.024 | Testis | AA = 206 AT = 102 |
| | | | | TT = 10 |
| rs449647 | APOE | 0.033 | Colon - sigmoid | AA = 206 AT = 102 |
| | | | | TT = 10 |
| rs405509 | APOE | 0.047 | Brain - hippocampus | TT = 33 TG = 109 |
| | | | | GG = 63 |
| rs405509 | APOE | 0.018 | Brain - cortex | TT = 33 TG = 109 |
| | | | | GG = 63 |

*Calculated using from Gene Tissue Expression calculator, https://gtexportal.org/home/testyourown

While APOE expression is important in understanding transcriptional regulation, it is not practical to measure APOE gene or protein expression in tissues from a large number of individuals, so it was asked if the finding can be extended to plasma APOE level, a more feasible clinical measurement. A Danish study was looked to that measured plasma APOE of 106 652 individuals (21). The results show a significant (P<10-6) association of SNP variants, including rs449647 in AANCR with plasma APOE level. Together, the genetic data confirm the role of AANCR in regulating the transcription of APOE. The genetic variants in AANCR that contribute to individual differences in APOE expression also affect risk of developing Alzheimer's disease.

Next, it was assessed if polymorphisms in AANCR contribute additional risk factors to Alzheimer's disease beyond the ε4 variant in APOE. Despite the proximity of the region of AANCR to APOE, the extent of linkage disequilibrium is modest. From the different populations studied by the HapMap Consortium (69), the r2 of rs449647 (in ANNCR) and rs429358 (in APOE) are 0.054 in Western Europeans (CEU), 0.068 in Yoruban from Idaban (YRI), 0.10 in Japanese from Tokyo (JPT) and 0.08 in Han Chinese from Beijing (CHB). Thus, the allelic associations of the SNPs in the AANCR regions with APOE expression and Alzheimer's disease are most likely independent of those of the F4 allele of rs429358. This shows that by regulating APOE expression, AANCR is a modifier of Alzheimer's disease. Transcription regulation by R-loops with modified RNA is common and prevalent

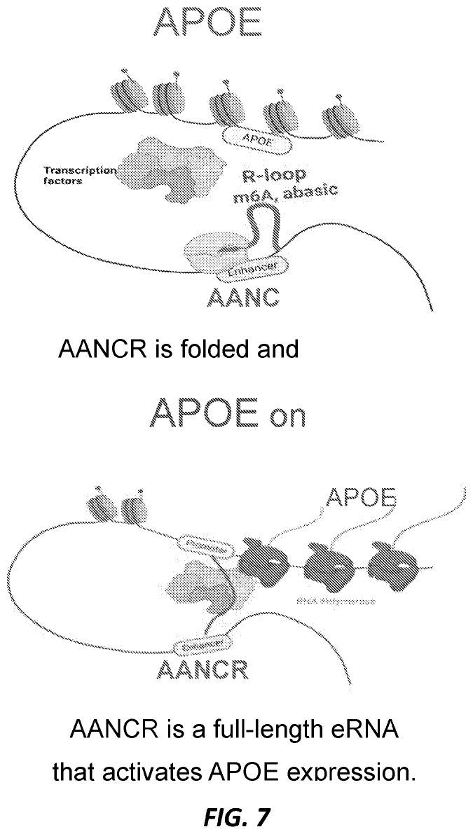

As shown above, R-loops with RNA abasic sites regulate AANCR transcription by pausing the elongating RNA Pol II. In addition to AANCR, 1,966 noncoding RNAs were identified that are regulated by this mechanism. In these transcripts, R-loops form, and in the RNAs, the adenosines are methylated (FIG. 6A, left) and then MPG likely removes the N6-methyladenosine to form abasic sites (FIG. 6A, middle panel). The resultant R-loops then pause the elongating RNA Pol II (FIG. 6A, right panel) such that the densities of the RNA Pol II 5′ to the R-loops are at least three times higher (pausing index >3) compared to the rest of the noncoding transcripts. This mechanism regulates RNA Pol II elongation of the 1,966 noncoding transcripts in all three cell types that were have studied, B cells (median PI=14.5), primary fibroblasts (median PI=5.6), and renal proximal tubule cells (median PI=6.3). FIG. 6B shows three examples of these non-coding transcripts with paused elongating RNA Pol II in B-cells, fibroblasts, and renal proximal tubule cells. Unlike the RNA Pol II that pauses in the promoter, the elongating RNA Pol II is not paused by the NELF protein complexes. FIG. 6C shows that the NELF protein complex is found behind (5′) the paused RNA Pol II while the R-loops are found ahead (3′; FIG. 6C) to the paused RNA Pol II. Like AANCR, the noncoding RNAs regulated by this mechanism are enhancers characterized by H3K27ac and H3K4me1 histone marks (FIGS. 6D and E). Together a regulatory mechanism was identified where RNA modifications stabilize R-loops to pause transcription elongation of enhancer RNAs.

### Discussion

In this study, it was shown that R-loops with RNA abasic sites regulate transcription elongation. This is the first identification of the regulation of RNA Pol II elongation by RNA sequence and structure. Although there have been tremendous advances in the understanding of RNA, we are still a long way from knowing its components and likely most of its function. RNA was simply considered an essential intermediary between DNA instructions and protein synthesis. It is now known that RNA is much more than a go-between for DNA and protein, and that RNA carries important regulatory information. RNAs are also more varied than originally thought. Nearly every nucleotide in DNA is transcribed into RNA (70,71), so in addition to mRNA and tRNA, there are myriad noncoding RNAs (72-74). Co-transcriptionally and post-transcriptionally,

the bases and sugar of RNA are modified, and the transcripts are spliced and polyadenylated with different lengths of adenosine. These processing steps add to the complexity of RNA by generating different transcripts from the same DNA template. The processing of RNA can differ by cell type and cellular environments resulting in a vast array of RNA species with different regulatory roles.

When RNA hybridizes with DNA to form R-loops, the RNA is exposed to enzymes that modify its bases including MPG which cleaves the glycosidic bond leading to RNA abasic sites. The discovery of DNA abasic sites and the enzymes such as methylpurine glycosylase which form and process abasic sites, led to the elucidation of base excision repair in response to DNA damage (75). However, the abasic sites in RNA differ from those in DNA due to the additional hydroxyl group. Studies have shown that RNA with abasic sites is more stable than DNA with abasic sites, as cleavage at the abasic site in RNA is at least 17 times less likely than at the abasic site in DNA (76). RNA abasic sites are rather abundant; by mass spectrometry, it was found that there are about 4 RNA abasic sites per million ribonucleotides (18), so with over 50 billion ribonucleotides in a cell, there are hundreds of thousands of RNA abasic sites in each cell. Many thousands of R-loops have also been mapped genome-wide (77-80).

Herein it was shown how R-loops with RNA abasic sites regulate the transcription of noncoding RNA. Herein the mechanism that regulates AANCR is detailed, the enhancer of APOE. In cells such as hepatocytes and astrocytes, AANCR is transcribed into a full-length enhancer

that activates APOE expression. In other cells, AANCR is not fully synthesized so APOE is not expressed. In these cells, the transcription and function of AANCR are dynamically regulated by RNA sequence modification and R-loops. The nascent RNA of AANCR forms R-loops, and in the R-loops, the RNA is modified by N6-adenine methylation and then the resultant N6-methyladenosines are likely removed by methylpurine glycosylase to become abasic sites that stabilize the R-loops. The stabilized R-loops pause RNA Pol II and prevent the synthesis of the full-length enhancer RNA, yet upon hypertonic stress, the R-loops resolve, and the enhancer RNA is fully transcribed to allow rapid activation of APOE. The nucleic-acid-mediated pausing keeps AANCR poised for rapid transcriptional responses. Like the hammerhead ribozyme whose catalytic function is dependent on its structure (81,82), AANCR is also dependent on its structure to function as an enhancer of APOE and APOC1 expression.

Beyond AANCR, this mechanism where RNA modifications and R-loops regulate transcription elongation determines the synthesis of over 1,000 noncoding RNA in three cell types. RNA Pol II pausing in the proximal promoter region mediated by protein complexes NELF and DSIF is well characterized (83-85). The RNA Pol II pauses identified in this study are different from the pauses in the promoter, they occur in the gene bodies and are mediated by R-loops that are stabilized by RNA abasic sites and not by pausing protein complexes. These R-loops regulate the transcription of enhancer RNAs thus they have a broad impact on gene expression.

In studying the RNA abasic sites and R-loops in AANCR, the mechanism that activates APOE transcription and expression was revealed. Previously, the induction of APOE in response to lipid load is well characterized, it involves the 3′ enhancer elements (86) and LXR/RXR signaling (87). Here, it was shown how cell-type and stress responsive expression of APOE are regulated. Understanding the regulation of APOE expression is critical given its role in lipid transport (88), maintenance of cognitive function (63,89,90), and response to infection (91,92) as well as immunotherapies (93,94). This study shows that sequence variants in AANCR explain individual variation in APOE expression and APOE plasma level. The variants in the AANCR region that affect APOE level also confer susceptibility to Alzheimer's disease. Based on the extent of linkage disequilibrium between the variants in AANCR and APOE, the effect of polymorphisms in AANCR is independent of the risk conferred by the APOE ε4 allele. By regulating APOE expression, AANCR acts as a modifier of Alzheimer's disease.

Genetic studies of Alzheimer's disease found that the A-allele of −491 SNP (rs449647) is the risk allele (64,95) and in the GTEx data, the A-allele is associated with lower APOE expression in different cell types including cells in the hippocampus. While studies have shown that Alzheimer's patients have low plasma APOE levels (21,96), other studies report more complex findings including higher APOE levels in the patients (97-100).

In conclusion, a regulatory mechanism was identified where the METTL3/METTL14 protein complex coalesces with methylpurine glycosylase forming N6-methyladenosines which are then cleaved resulting in RNA abasic sites on R-loops to pause RNA Pol II transcription. Over 1,000 noncoding RNAs were identified that are regulated by this mechanism and it was detailed how this pathway regulates the expression of APOE. It was surmised that RNA modification and RNA structure play a role in the dynamic regulation of gene expression.

### BIBLIOGRAPHY

1. Cheung, V. G., Spielman, R. S., Ewens, K. G., Weber, T. M., Morley, M. and Burdick, J. T. (2005) Mapping determinants of human gene expression by regional and genome-wide association. Nature, 437, 1365-1369.
2. Morley, M., Molony, C. M., Weber, T. M., Devlin, J. L., Ewens, K. G., Spielman, R. S. and Cheung, V. G. (2004) Genetic analysis of genome-wide variation in human gene expression. Nature, 430, 743-747.
3. Boccaletto, P., Machnicka, M. A., Purta, E., Piatkowski, P., Baginski, B., Wirecki, T. K., de Cr'ecy-Lagard, V., Ross, R., Limbach, P. A., Kotter, A. et al. (2018) MODOMICS: a database of RNA modification pathways. 2017 update. Nucleic. Acids. Res., 46, D303-D307.
4. Mannion, N., Arieti, F., Gallo, A., Keegan, L. P. and O'Connell, M. A. (2015) New insights into the biological role of mammalian ADARs; the RNA editing proteins. Biomolecules, 5, 2338-2362.
5. Stefl, R., Oberstrass, F. C., Hood, J. L., Jourdan, M., Zimmermann, M., Skrisovska, L., Maris, C., Peng, L., Hofr, C., Emeson, R. B. et al. (2010) The solution structure of the ADAR2 dsRBM-RNA complex reveals a sequence-specific readout of the minor groove. Cell, 143, 225-237.
6. Schirle, N. T., Sheu-Gruttadauria, J. and MacRae, I. J. (2014) Structural basis for microRNA targeting. Science, 346, 608-613.
7. Chen, Y.-Z., Bennett, C. L., Huynh, H. M., Blair, I. P., Puls, I., Irobi, J., Dierick, I., Abel, A., Kennerson, M. L., Rabin, B. A. et al. (2004) DNA/RNA helicase gene mutations in a form of juvenile amyotrophic lateral sclerosis (ALS4). Am. J. Hum. Genet., 74, 1128-1135.
8. Grunseich, C., Wang, I. X., Watts, J. A., Burdick, J. T., Guber, R. D., Zhu, Z., Bruzel, A., Lanman, T., Chen, K., Schindler, A. B. et al. (2018) Senataxin mutation reveals how R-Loops promote transcription by blocking DNA methylation at gene promoters. Mol. Cell, 69, 426-437.
9. Grunseich, C., Patankar, A., Amaya, J., Watts, J. A., Li, D., Ramirez, P., Schindler, A. B., Fischbeck, K. H. and Cheung, V. G. (2020) Clinical and molecular aspects of senataxin mutations in ALS4. Ann. Neurol., 87, 547-555.
10. Aguilera, A. and Garcia-Muse, T. (2012) R loops: from transcription byproducts to threats to genome stability. Mol. Cell, 46, 115-124.
11. Castillo-Guzman, D. and Ch'edin, F. (2021) Defining R-loop classes and their contributions to genome instability. DNA Repair (Amst.), 106, 103182.
12. Niehrs, C. and Luke, B. (2020) Regulatory R-loops as facilitators of gene expression and genome stability. Nat. Rev. Mol. Cell Biol., 21, 167-178.
13. Thomas, M., White, R. L. and Davis, R. W. (1976) Hybridization of RNA to double-stranded DNA: formation of R-loops. Proc. Natl. Acad. Sci. U.S.A., 73, 2294-2298.
14. Cristini, A., Groh, M., Kristiansen, M. S. and Gromak, N. (2018) RNA/DNA hybrid interactome identifies DXH9 as a molecular player in transcriptional termination and R-Loop-Associated DNA damage. Cell Rep., 23, 1891-1905.
15. Wang, I. X., Grunseich, C., Fox, J., Burdick, J., Zhu, Z., Ravazian, N., Hafner, M. and Cheung, V. G. (2018) Human proteins that interact with RNA/DNA hybrids. Genome Res., 28, 1405-1414.
16. Yan, Q., Wulfridge, P., Doherty, J., Fernandez-Luna, J. L., Real, P. J., Tang, H.-Y. and Sarma, K. (2022) Proximity labeling identifies a repertoire of site-specific R-loop modulators. Nat. Commun., 13, 53.
17. Lindahl, T. (1974) An N-glycosidase from Escherichia coli that releases free uracil from DNA containing deaminated cytosine residues. Proc. Natl. Acad. Sci. U.S.A., 71, 3649-3653.
18. Liu, Y., Rodriguez, Y., Ross, R. L., Zhao, R., Watts, J. A., Grunseich, C., Bruzel, A., Li, D., Burdick, J. T., Prasad, R. et al. (2020) RNA abasic sites in yeast and human cells. Proc. Natl. Acad. Sci. U.S.A., 117, 20689-20695.
19. Zhang, Y., Sloan, S. A., Clarke, L. E., Caneda, C., Plaza, C. A., Blumenthal, P. D., Vogel, H., Steinberg, G. K., Edwards, M. S. B., Li, G. et al. (2016) Purification and characterization of progenitor and mature human astrocytes reveals transcriptional and functional differences with mouse. Neuron, 89, 37-53.
20. Wu, T. D. and Nacu, S. (2010) Fast and SNP-tolerant detection of complex variants and splicing in short reads. Bioinformatics, 26, 873-881.
21. Rasmussen, K. L., Tybjorg-Hansen, A., Nordestgaard, B. G. and Frikke-Schmidt, R. (2018) Plasma apolipoprotein e levels and risk of dementia: a mendelian randomization study of 106, 562 individuals. Alzheimers Dement, 14, 71-80.
22. Carpentier, A., Tesfaye, A., Chu, V., Nimgaonkar, I., Zhang, F., Lee, S. B., Thorgeirsson, S. S., Feinstone, S. M. and Liang, T. J. (2014) Engrafted human stem cell-derived hepatocytes establish an infectious HCV murine model. J. Clin. Invest., 124, 4953-4964.
23. Adhikari, S., Manthena, P. V., Uren, A. and Roy, R. (2008) Expression, purification and characterization of codon-optimized human N-methylpurine-DNA glycosylase from Escherichia coli. Protein Expr. Purif., 58, 257-262.
24. Ryder, S. P., Recht, M. I. and Williamson, J. R. (2008) Quantitative analysis of protein-RNA interactions by gel mobility shift. Methods Mol. Biol., 488, 99-115.
25. Kwak, H., Fuda, N. J., Core, L. J. and Lis, J. T. (2013) Precise maps of RNA polymerase reveal how promoters direct initiation and pausing. Science, 339, 950-953.
26. Watts, J. A., Burdick, J., Daigneault, J., Zhu, Z., Grunseich, C., Bruzel, A. and Cheung, V. G. (2019) Cis elements that mediate RNA polymerase II pausing regulate human gene expression. Am. J. Hum. Genet., 105, 677-688.
27. Kristj\acute{ }ansd\acute{ } ottir, K., Dziubek, A., Kang, H. M. and Kwak, H. (2020) Population-scale study of eRNA transcription reveals bipartite functional enhancer architecture. Nat. Commun., 11, 5963.
28. Miller, H. E., Montemayor, D., Li, J., Levy, S., Pawar, R., Hartono, S., Sharma, K., Frost, B., Chedin, F. and Bishop, A. J. R. (2022) Exploration and analysis of R-loop mapping data with RLBase. Nucleic Acids Res., gkac732.
29. Ramirez, P., Crouch, R. J., Cheung, V. G. and Grunseich, C. (2021) R-Loop analysis by dot-blot. J. Vis. Exp., https.//doi.org/10.3791/62069.
30. Bou-Nader, C., Bothra, A., Garboczi, D. N., Leppla, S. H. and Zhang, J. (2022) Structural basis of R-loop recognition by the S9.6 monoclonal antibody. Nat. Commun., 13, 1641.
31. Hu, Z., Zhang, A., Storz, G., Gottesman, S. and Leppla, S. H. (2006) An antibody-based microarray assay for small RNA detection. Nucleic Acids Res., 34, e52.
32. Kosar, M., Piccini, D., Foiani, M. and Giannattasio, M. (2021) A rapid method to visualize human mitochondrial DNA replication through rotary shadowing and transmission electron microscopy. Nucleic Acids Res., 49, e121.
33. Yamaguchi, Y., Takagi, T., Wada, T., Yano, K., Furuya, A., Sugimoto, S., Hasegawa, J. and Handa, H. (1999) NELF, a multisubunit complex containing RD, cooperates with DSIF to repress RNA polymerase II elongation. Cell, 97, 41-51.
34. Stork, C. T., Bocek, M., Crossley, M. P., Sollier, J., Sanz, L. A., Ch\acute{ }edin, F., Swigut, T. and Cimprich, K. A. (2016) Co-transcriptional R-loops are the main cause of estrogen-induced DNA damage. Elife, 5, 17548.
35. Frye, M., Jaffrey, S. R., Pan, T., Rechavi, G. and Suzuki, T. (2016) RNA modifications: what have we learned and where are we headed?Nat. Rev. Genet., 17, 365-372.
36. Pan, T. (2013) N6-methyl-adenosine modification in messenger and long non-coding RNA. Trends Biochem. Sci, 38, 204-209.
37. Shi, H., Wei, J. and He, C. (2019) Where, when, and how: context-dependent functions of RNA methylation writers, readers, and erasers. Mol. Cell, 74, 640-650.
38. Zaccara, S., Ries, R. J. and Jaffrey, S. R. (2019) Reading, writing and erasing mRNA methylation. Nat. Rev. Mol. Cell Biol., 20, 608-624.
39. Abakir, A., Giles, T. C., Cristini, A., Foster, J. M., Dai, N., Starczak, M., Rubio-Roldan, A., Li, M., Eleftheriou, M., Crutchley, J. et al. (2020) N6-methyladenosine regulates the stability of RNA:DNA hybrids in human cells. Nat. Genet., 52, 48-55.
40. Kang, H. J., Cheon, N. Y., Park, H., Jeong, G. W., Ye, B. J., Yoo, E. J., Lee, J. H., Hur, J.-H., Lee, E.-A., Kim, H. et al. (2021) TonEBP recognizes R-loops and initiates m6A RNA methylation for R-loop resolution. Nucleic Acids Res., 49, 269-284.
41. Zhang, C., Chen, L., Peng, D., Jiang, A., He, Y., Zeng, Y., Xie, C., Zhou, H., Luo, X., Liu, H. et al. (2020) METTL3 and N6-Methyladenosine promote homologous recombination-mediated repair of DSBs by modulating DNA-RNA hybrid accumulation. Mol. Cell, 79, 425-442.
42. Harper, J. E., Miceli, S. M., Roberts, R. J. and Manley, J. L. (1990) Sequence specificity of the human mRNA N6-adenosine methylase in vitro. Nucleic Acids Res., 18, 5735-5741.
43. Csepany, T., Lin, A., Baldick, C. J. and Beemon, K. (1990) Sequence specificity of mRNA N6-adenosine methyltransferase. J. Biol. Chem., 265, 20117-20122.
44. Liu, J., Yue, Y., Han, D., Wang, X., Fu, Y., Zhang, L., Jia, G., Yu, M., Lu, Z., Deng, X. et al. (2014) A METTL3-METTL14 complex mediates mammalian nuclear RNA N6-adenosine methylation. Nat. Chem. Biol., 10, 93-95.
45. Wang, X., Feng, J., Xue, Y., Guan, Z., Zhang, D., Liu, Z., Gong, Z., Wang, Q., Huang, J., Tang, C. et al. (2016) Structural basis of N(6)-adenosine methylation by the METTL3-METTL14 complex.
Nature, 534, 575-578.
46. Tome, J. M., Tippens, N. D. and Lis, J. T. (2018) Single-molecule nascent RNA sequencing reveals regulatory domain architecture at promoters and enhancers. Nat. Genet., 50, 1533-1541.
47. Core, L. J., Martins, A. L., Danko, C. G., Waters, C. T., Siepel, A. and Lis, J. T. (2014) Analysis of nascent RNA identifies a unified architecture of initiation regions at mammalian promoters and enhancers. Nat. Genet., 46, 1311-1320.
48. Mahat, D. B., Kwak, H., Booth, G. T., Jonkers, I. H., Danko, C. G., Patel, R. K., Waters, C. T., Munson, K., Core, L. J. and Lis, J. T. (2016) Base-Pair resolution genome-wide mapping of active RNA polymerases using precision nuclear run-on (PRO-seq). Nat. Protoc., 11, 1455-1476.
49. Gish, W. and States, D. J. (1993) Identification of protein coding regions by database similarity search. Nat. Genet., 3, 266-272.
50. Bateman, A., Coin, L., Durbin, R., Finn, R. D., Hollich, V., Griffiths-Jones, S., Khanna, A., Marshall, M., Moxon, S., Sonnhammer, E. L. L. et al. (2004) The pfam protein families database. Nucleic Acids Res., 32, D138-D141.
51. Guo, J.-C., Fang, S.-S., Wu, Y., Zhang, J.-H., Chen, Y., Liu, J., Wu, B., Wu, J.-R., Li, E.-M., Xu, L.-Y. et al. (2019) CNIT: a fast and accurate web tool for identifying protein-coding and long non-coding transcripts based on intrinsic sequence composition. Nucleic Acids Res., 47, W516-W522.
52. Kong, L., Zhang, Y., Ye, Z.-Q., Liu, X.-Q., Zhao, S.-Q., Wei, L. and Gao, G. (2007) CPC: assess the protein-coding potential transcripts using sequence features and support vector machine. Nucleic Acids Res., 35, W345-W349.
53. Wang, L., Park, H. J., Dasari, S., Wang, S., Kocher, J.-P. and Li, W. (2013) CPAT: coding-potential assessment tool using an alignment-free logistic regression model. Nucleic Acids Res., 41, e74.
54. Paulsen, M. T., Veloso, A., Prasad, J., Bedi, K., Ljungman, E. A., Tsan, Y.-C., Chang, C.-W., Tarrier, B., Washburn, J. G., Lyons, R. et al. (2013) Coordinated regulation of synthesis and stability of RNA during the acute TNF-induced proinflammatory response. Proc. Natl. Acad. Sci. U.S.A., 110, 2240-2245.
55. Davis, C. A., Hitz, B. C., Sloan, C. A., Chan, E. T., Davidson, J. M., Gabdank, I., Hilton, J. A., Jain, K., Baymuradov, U. K., Narayanan, A. K. et al. (2018) The encyclopedia of DNA elements (ENCODE): data portal update. Nucleic Acids Res., 46, D794-D801.
56. Moore, J. E., Purcaro, M. J., Pratt, H. E., Epstein, C. B., Shoresh, N., Adrian, J., Kawli, T., Davis, C. A., Dobin, A., Kaul, R. et al. (2020) Expanded encyclopaedias of DNA elements in the human and mouse genomes. Nature, 583, 699-710.
57. Fishilevich, S., Nudel, R., Rappaport, N., Hadar, R., Plaschkes, I., Iny Stein, T., Rosen, N., Kohn, A., Twik, M., Safran, M. et al. (2017) GeneHancer: genome-wide integration of enhancers and target genes in genecards. Database (Oxford), 2017, bax028.
58. Heintzman, N. D., Stuart, R. K., Hon, G., Fu, Y., Ching, C. W., Hawkins, R. D., Barrera, L. O., Van Calcar, S., Qu, C., Ching, K. A. et al. (2007) Distinct and predictive chromatin signatures of transcriptional promoters and enhancers in the human genome. Nat. Genet., 39, 311-318.
59. Heintzman, N. D., Hon, G. C., Hawkins, R. D., Kheradpour, P., Stark, A., Harp, L. F., Ye, Z., Lee, L. K., Stuart, R. K., Ching, C. W. et al. (2009) Histone modifications at human enhancers reflect global cell-type-specific gene expression. Nature, 459, 108-112.
60. Krietenstein, N., Abraham, S., Venev, S. V., Abdennur, N., Gibcus, J., Hsieh, T.-H. S., Parsi, K. M., Yang, L., Maehr, R., Mirny, L. A. et al. (2020) Ultrastructural details of mammalian chromosome architecture. Mol. Cell, 78, 554-565.
61. Obayashi, T., Kagaya, Y., Aoki, Y., Tadaka, S. and Kinoshita, K. (2019) COXPRESdb v7: a gene coexpression database for 11 animal species supported by 23 coexpression platforms for technical evaluation and evolutionary inference. Nucleic Acids Res., 47, D55-D62.
62. Kodama, Y., Mashima, J., Kosuge, T., Kaminuma, E., Ogasawara, O., Okubo, K., Nakamura, Y. and Takagi, T. (2018) DNA data bank of japan: 30th anniversary. Nucleic Acids Res., 46, D30-D35.
63. Corder, E. H., Saunders, A. M., Strittmatter, W. J., Schmechel, D. E., Gaskell, P. C., Small, G. W., Roses, A. D., Haines, J. L. and Pericak-Vance, M. A. (1993) Gene dose of apolipoprotein e type 4 allele and the risk of alzheimer's disease in late onset families. Science, 261, 921-923.
64. Bullido, M. J., Artiga, M. J., Recuero, M., Sastre, I., Garc\acute{ }ia, M. A., Aldudo, J., Lendon, C., Han, S. W., Morris, J. C., Frank, A. et al. (1998) A polymorphism in the regulatory region of APOE associated with risk for alzheimer's dementia. Nat. Genet., 18, 69-71.
65. Lambert, J. C., Berr, C., Pasquier, F., Delacourte, A., Frigard, B., Cottel, D., P\acute{ }erez-Tur, J., Mouroux, V., Mohr, M., C\acute{ }ecyre, D. et al. (1998) Pronounced impact of Thl/E47cs mutation compared with −491 AT mutation on neural APOE gene expression and risk of developing alzheimer's disease. Hum. Mol. Genet., 7, 1511-1516.
66. Wang, J. C., Kwon, J. M., Shah, P., Morris, J. C. and Goate, A. (2000) Effect of APOE genotype and promoter polymorphism on risk of alzheimer's disease. Neurology, 55, 1644-1649.
67. Marioni, R. E., Harris, S. E., Zhang, Q., McRae, A. F., Hagenaars, S. P., Hill, W. D., Davies, G., Ritchie, C. W., Gale, C. R., Starr, J. M. et al. (2018) GWAS on family history of alzheimer's disease. Transl Psychiatry, 8, 99.
68. Ardlie, K. G., DeLuca, D. S., Segre, A. V., Sullivan, T. J., Young, T. R., Gelfand, E. T., Trowbridge, C. A., Maller, J. B., Tukiainen, T., Lek, M. et al. (2015) The genotype-tissue expression (GTEx) pilot analysis: multitissue gene regulation in humans. Science, 348, 648-660.
69. Consortium, International HapMap, Frazer, K. A., Ballinger, D. G., Cox, D. R., Hinds, D. A., Stuve, L. L., Gibbs, R. A., Belmont, J. W., Boudreau, A., Hardenbol, P. et al. (2007) A second generation human haplotype map of over 3.1 million SNPs. Nature, 449, 851-861.
70. Project Consortium, ENCODE, Birney, E., Stamatoyannopoulos, J. A., Dutta, A., Guig\acute{ }o, R., Gingeras, T. R., Margulies, E. H., Weng, Z., Snyder, M., Dermitzakis, E. T. et al. (2007) Identification and analysis of functional elements in 1% of the human genome by the ENCODE pilot project. Nature, 447, 799-816.
71. Hangauer, M. J., Vaughn, I. W. and McManus, M. T. (2013) Pervasive transcription of the human genome produces thousands of previously unidentified long intergenic noncoding RNAs. PLoS
Genet., 9, e1003569.
72. Cech, T. R. and Steitz, J. A. (2014) The noncoding RNA revolution-trashing old rules to forge new ones. Cell, 157, 77-94.
73. Mattick, J. S. (2018) The state of long non-coding RNA biology. Noncoding RNA, 4, E17.
74. Mercer, T. R., Dinger, M. E. and Mattick, J. S. (2009) Long non-coding RNAs: insights into functions. Nat. Rev. Genet., 10, 155-159.
75. Lindahl, T. (1993) Instability and decay of the primary structure of DNA. Nature, 362, 709-715.
76. K\ddot{ }upfer, P. A. and Leumann, C. J. (2007) The chemical stability of abasic RNA compared to abasic DNA. Nucleic. Acids. Res., 35, 58-68.
77. Crossley, M. P., Bocek, M. J., Hamperl, S., Swigut, T. and Cimprich, K. A. (2020) qDRIP: a method to quantitatively assess RNA-DNA hybrid formation genome-wide. Nucleic. Acids. Res., 48, e84.
78. Ginno, P. A., Lott, P. L., Christensen, H. C., Korf, I. and Ch\acute{ }edin, F. (2012) R-loop formation is a distinctive characteristic of unmethylated human CpG island promoters. Mol. Cell, 45, 814-825.
79. Sanz, L. A., Hartono, S. R., Lim, Y. W., Steyaert, S., Rajpurkar, A., Ginno, P. A., Xu, X. and Ch\acute{ }edin, F. (2016) Prevalent, dynamic, and conserved R-Loop structures associate with specific epigenomic signatures in mammals. Mol. Cell, 63, 167-178.
80. Wahba, L., Costantino, L., Tan, F. J., Zimmer, A. and Koshland, D. (2016) Si-DRIP-seq identifies high expression and polyA tracts as major contributors to R-loop formation. Genes Dev., 30, 1327-1338.
81. Pley, H. W., Flaherty, K. M. and McKay, D. B. (1994) Three-dimensional structure of a hammerhead ribozyme. Nature, 372, 68-74.
82. Scott, W. G., Finch, J. T. and Klug, A. (1995) The crystal structure of an all-RNA hammerhead ribozyme: a proposed mechanism for RNA catalytic cleavage. Cell, 81, 991-1002.
83. Gilchrist, D. A., Nechaev, S., Lee, C., Ghosh, S. K. B., Collins, J. B., Li, L., Gilmour, D. S. and Adelman, K. (2008) NELF-mediated stalling of pol II can enhance gene expression by blocking promoter-proximal nucleosome assembly. Genes Dev., 22, 1921-1933.
84. Vos, S. M., Farnung, L., Urlaub, H. and Cramer, P. (2018) Structure of paused transcription complex pol II-DSIF-NELF. Nature, 560, 601-606.
85. Wu, C.-H., Yamaguchi, Y., Benjamin, L. R., Horvat-Gordon, M., Washinsky, J., Enerly, E., Larsson, J., Lambertsson, A., Handa, H. and Gilmour, D. (2003) NELF and DSIF cause promoter proximal pausing on the hsp70 promoter in drosophila. Genes Dev., 17, 1402-1414.
86. Shih, S.-J., Allan, C., Grehan, S., Tse, E., Moran, C. and Taylor, J. M. (2000) Duplicated downstream enhancers control expression of the human apolipoprotein e gene in macrophages and adipose tissue. J. Biol. Chem., 275, 31567-31572.
87. Laffitte, B. A., Repa, J. J., Joseph, S. B., Wilpitz, D. C., Kast, H. R., Mangelsdorf, D. J. and Tontonoz, P. (2001) LXRs control lipid-inducible expression of the apolipoprotein e gene in macrophages and adipocytes. Proc. Natl. Acad. Sci. U.S.A., 98, 507-512.
88. Mahley, R. W. (1988) Apolipoprotein E: cholesterol transport protein with expanding role in cell biology. Science, 240, 622-630.
89. Roses, A. D. (1996) Apolipoprotein e alleles as risk factors in alzheimer's disease. Annu. Rev. Med., 47, 387-400.
90. Mahley, R. W. and Huang, Y. (2012) Apolipoprotein e sets the stage: response to injury triggers neuropathology. Neuron, 76, 871-885.
91. Jiang, J. and Luo, G. (2009) Apolipoprotein e but not b is required for the formation of infectious hepatitis c virus particles. J. Virol., 83, 12680-12691.
92. Kuo, C.-L., Pilling, L. C., Atkins, J. L., Masoli, J. A. H., Delgado, J., Kuchel, G. A. and Melzer, D. (2020) APOE e4 genotype predicts severe COVID-19 in the UK biobank community cohort. J.
Gerontol. A, 75, 2231-2232.
93. Pencheva, N., Buss, C. G., Posada, J., Merghoub, T. and Tavazoie, S. F. (2014) Broad-spectrum therapeutic suppression of metastatic melanoma through nuclear hormone receptor activation. Cell, 156, 986-1001.
94. Tavazoie, M. F., Pollack, I., Tanqueco, R., Ostendorf, B. N., Reis, B. S., Gonsalves, F. C., Kurth, I., Andreu-Agullo, C., Derbyshire, M. L., Posada, J. et al. (2018) LXR/ApoE activation restricts innate
immune suppression in cancer. Cell, 172, 825-840.
95. Lambert, J., Ibrahim-Verbaas, C., Harold, D., Naj, A., Sims, R., Bellenguez, C., DeStafano, A., Bis, J., Beecham, G., Grenier-Boley, B. et al. (2013) Meta-analysis of 74, 046 individuals identifies 11 new susceptibility loci for Alzheimer's disease. Nat. Genet., 45, 1452-1458.
96. Bertrand, P., Poirier, J., Oda, T., Finch, C. E. and Pasinetti, G. M. (1995) Association of apolipoprotein e genotype with brain levels of apolipoprotein e and apolipoprotein j (clusterin) in Alzheimer disease. Brain Res. Mol. Brain Res., 33, 174-178.
97. Koch, M., DeKosky, S. T., Goodman, M., Sun, J., Furtado, J. D., Fitzpatrick, A. L., Mackey, R. H., Cai, T., Lopez, O. L., Kuller, L. H. et al. (2020) Association of apolipoprotein e in lipoprotein subspecies with risk of dementia. JAMA Network Open, 3, e209250.
98. Laws, S. M., Hone, E., Taddei, K., Harper, C., Dean, B., McClean, C., Masters, C., Lautenschlager, N., Gandy, S. E. and Martins, R. N. (2002) Variation at the APOE −491 promoter locus is associated with altered brain levels of apolipoprotein E. Mol. Psychiatry, 7, 886-890.
99. Lehtim\ddot{ }aki, T., Pirttil\ddot{ }a, T., Mehta, P. D., Wisniewski, H. M., Frey, H. and Nikkari, T. (1995) Apolipoprotein e (apoE) polymorphism and its influence on ApoE concentrations in the cerebrospinal fluid in finnish patients with alzheimer's disease. Hum. Genet., 95, 39-42.
100. Taddei, K., Clarnette, R., E. Gandy, S. and Martins, R. N. (1997) Increased plasma apolipoprotein e (apoE) levels in alzheimer's disease. Neurosci. Lett., 223, 29-32.

### Example II—an Enhancer RNA of APOE and Alzheimer's Disease (AD)

APOE4 is a strong genetic risk factor for AD. Over 65% of AD patients have at least one APOE4 allele. APOE4 also a risk factor of Dementia with Lewy Bodies, TBI recovery, COVID-19 severity. For those that are APOE4 homozygotes: by the age of 85, 51% men and 60% women will develop AD.

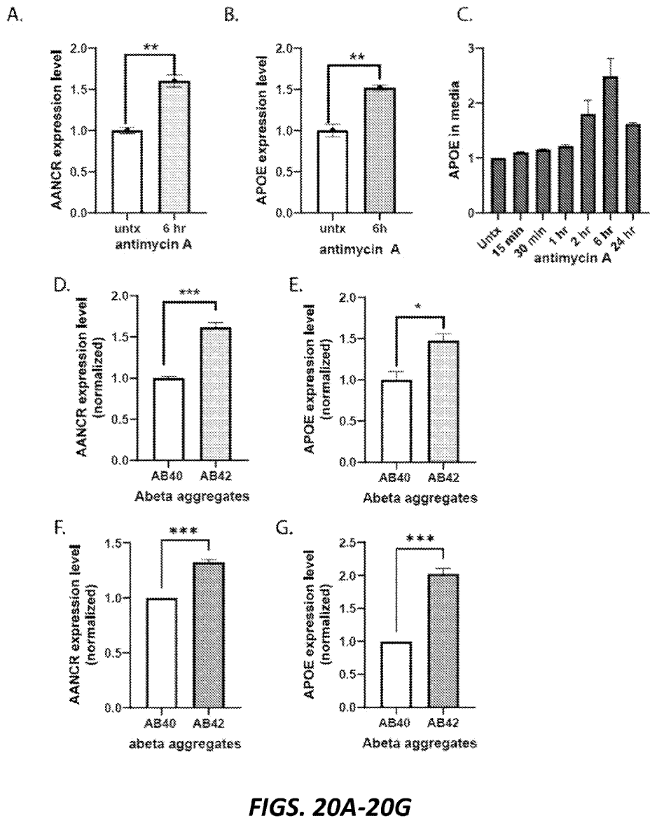

APOE4 and APOE3 differ by one nucleotide (FIG. 16A).

While the potential treatment for AD has varied from targeting/correcting the mutant protein to removing APOE with antibodies to RNA-based targeting, there is still is no effective treatment for AD. One concern is that APOE is a small gene. Further, APOE4 is too much of an aberrant protein—like a leak in a pipe. One could try to repair the pipe or one could just turn off the valve. Provided herein is a method to turn off the valve for APOE expression, by targeting the non-coding RNA AANCR (FIG. 16B).

As discussed above in Example I, R-loops are subjected to a variety of chemical modifications. It was found that R-loops are bound by enzymes that methylate the RNA (METTL3, METTL14, NSUN2), enzymes that remove bases from the RNA (MPG) or enzymes that cleave the phosphodiester backbone (APE1). MPG removes base from RNA, creating R-loops with abasic RNA. Since MPG makes R-loops with abasic RNA, when MPG was knocked down a strong induction of APOE both at the RNA and protein level occurred. A marked increase in APOE RNA synthesis occurred. Thus, loss of MPG increased the rate at which APOE was transcribed. It was found that the enzyme methylpurine glycosylase acts on RNA only if the RNA is found in an RNA/DNA hybrid as in an R-loop. APOE is silent in resting fibroblasts, so there is no RNA to form an R-loop, and R-loops were not found in APOE in resting fibroblasts. However, R-loops were found in the region upstream of APOE and looking at nascent transcription it was realized there is active transcription in the region upstream of APOE. Using immunoprecipitation followed by qPCR one can detect R-loop, bound MPG, and abasic RNA in the region upstream of APOE.

Previous work has shown estrogen increases R-loop abundance. As discussed above in Example I, cells were treated with estrogen and an increased R-loop abundance upstream of APOE was observed. Then, looking at nascent transcription after estrogen treatment there is a change in the pattern of transcription. There is more RNA polymerase II that accumulates in the region upstream of APOE. There is more RNA polymerase pausing which is quantified by the pausing index (PI). The increase of R-loop with abasic RNA creates a barrier to transcription, a roadblock, this barrier pauses the RNA polymerase II.

Why is there transcription upstream of APOE where an R-loop can form to create a barrier to pause the polymerase?Nascent transcription analysis shows there is more transcription in the intergenic region between TOMM40 and APOE. It was then realized that there is a non-coding RNA between TOMM40 and APOE. Methods show the RNA between TOMM40 and APOE does not encode a protein. Thus, there is a non-coding RNA upstream of APOE that now named AANCR: APOE associated non-coding RNA.

The non-coding RNA has a 5′ cap and there is a detectable transcription start site. It has histone modifications consistent with it being an enhancer RNA. There is also a DNAse hypersensitive site at the 5′ end, which also indicates an active promoter. By chromatin capture (Hi-C) it is shown that AANCR is physically associated with APOE and APOC1, but not TOMM40. Knockdown of MPG leads to increased expression of APOE and APOC1 but does not affect expression of TOMM40.

Hepatocytes and astrocytes express APOE, while lymphocyte and fibroblasts normally do not. In cell types where APOE is expressed (hepatocyte or HepG2), full length AANCR is expressed. The model shows there is no R-loop with abasic RNA, therefore the RNA polymerase II does not pause, and there is full length expression of AANCR. In cell types where APOE is silent (white cells or B-cells), full length AANCR is not expressed. The model shows an R-loop with abasic RNA and paused RNA Pol II. The presence of the R-loop with abasic RNA prevents synthesis of full length AANCR and APOE remains silent.

APOE is stress responsive, which is one answer as to why cells have this mechanism where dynamic tuning of R-loops can regulate APOE expression (to regulate gene expression). As shown above, in renal proximal tubule cells hypertonic stress results in higher expression of full length AANCR and a corresponding increase in APOE expression at the RNA level. There is increased protein expression. And increased secretion of APOE in the media. Taking conditioned media with higher APOE protein is protective against apoptosis when cells are exposed to hypertonic stress. APOE expression is stress responsive and it seems to be protective against cell stress.

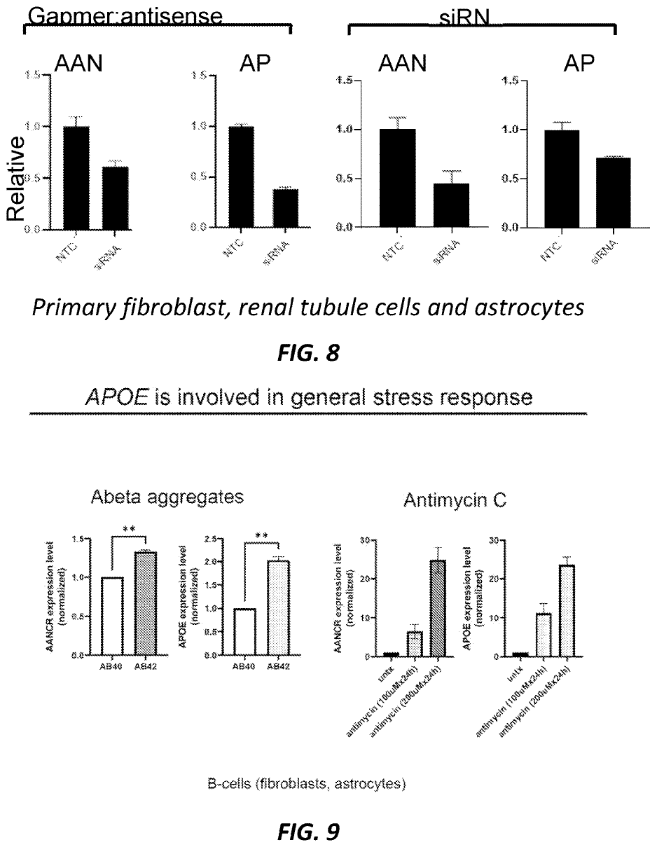

Thus, AANCER regulates stress responsive APOE expression; however, is this relevant to Alzheimer's disease (AD)?Genetic studies have linked polymorphisms upstream of APOE to Alzheimer's disease. The SNP alleles in AACNR are significantly associated with AD risk. There are SNPs in APOE that are also associated with AD risk. While AANCR is physically near APOE, the SNPs in AANCR are not in linkage disequilibrium with the SNPs in AD. That is, the SNPs in AANCR confer AD risk that is independent of the SNPs in APOE. This suggests SNPs in AANCR may modify the AD risk for SNPs in APOE. For example, SNPS in AANCR may lead to higher APOE. For someone who has the higher expressing form of AANCR and APOE4 allele, they may have higher risk of AD than the person with low expression form of AANCR and APOE4. Provided herein is model where AANCR with an R-loop with abasic sites does not induce APOE. AANCR without an R-loop allows for full length expression and activation of APOE (FIG. 7; Watts et al. 2022).

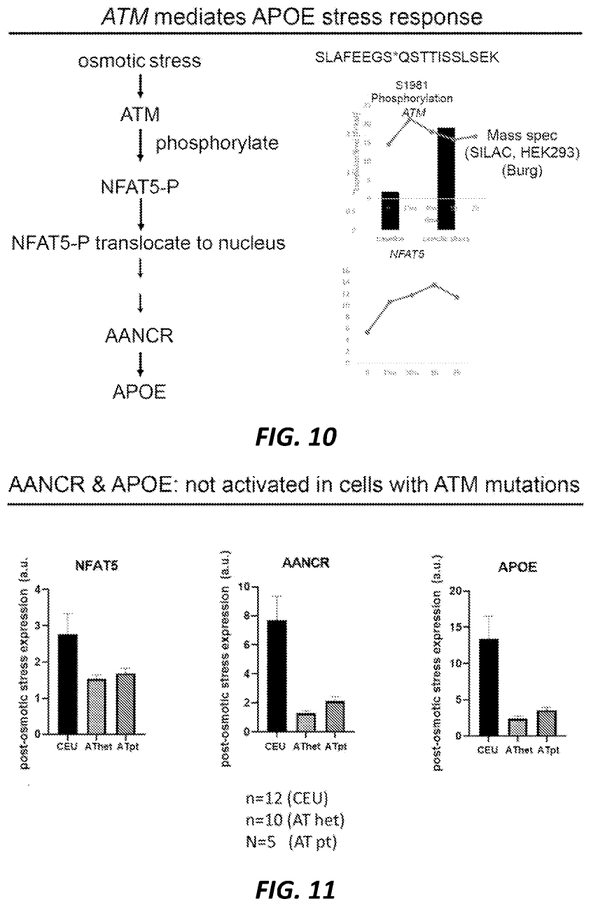

The model predicts that loss of AANCR expression will prevent expression of APOE. Knockdown of AANCR by siRNA or gapmer antisense oligonucleotides results in downregulation of APOE expression (FIG. 8). The siRNAs were as follows:

siAANCR #1
Sense:
(SEQ IDN NO: 47)
CCAUGCUCAAUACACGUUAtt
Antisense:
(SEQ IDN NO: 48)
UAACGUGUAUUGAGCAUGGag
siAANCR #2
Sense:
(SEQ IDN NO: 49)
GGAUUUAUAACAGGGCUUAtt
Antisense:
(SEQ IDN NO: 50)
UAAGCCCUGUUAUAAAUCCca
siAANCR #3
Sense:
(SEQ IDN NO: 51)
UCCUAACCUUAACCCAGAAtt
Antisense:
(SEQ IDN NO: 52)
UUCUGGGUUAAGGUUAGGAtt
siRNA_AP1
Antisense
(SEQ IDN NO: 53)
CTGTCTCTGAGTGCAGAGATG

This confirms targeting AANCR can reduce APOE expression.

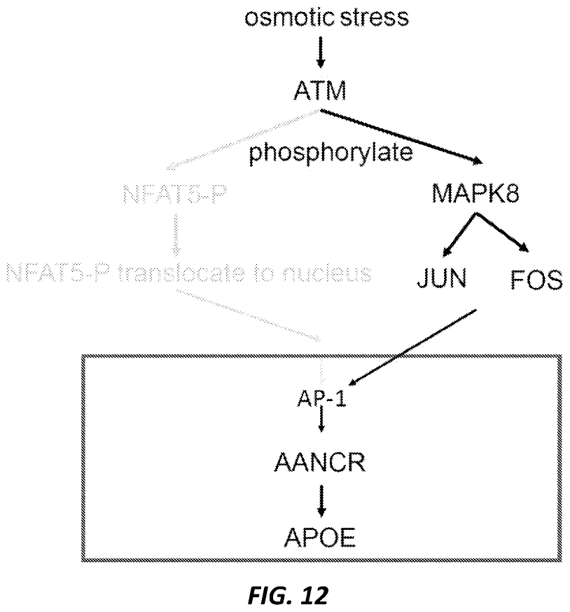

It was also tested if AANCR responds to stresses beyond osmotic stress. Treating cells with amyloid beta aggregates, the aggregate that forms in AD, results in increase AANCR expression and increased APOE expression (FIG. 9). Similar for oxidative phosphorylation inhibitor (mitochondrial stress) antimycin C (FIG. 9).

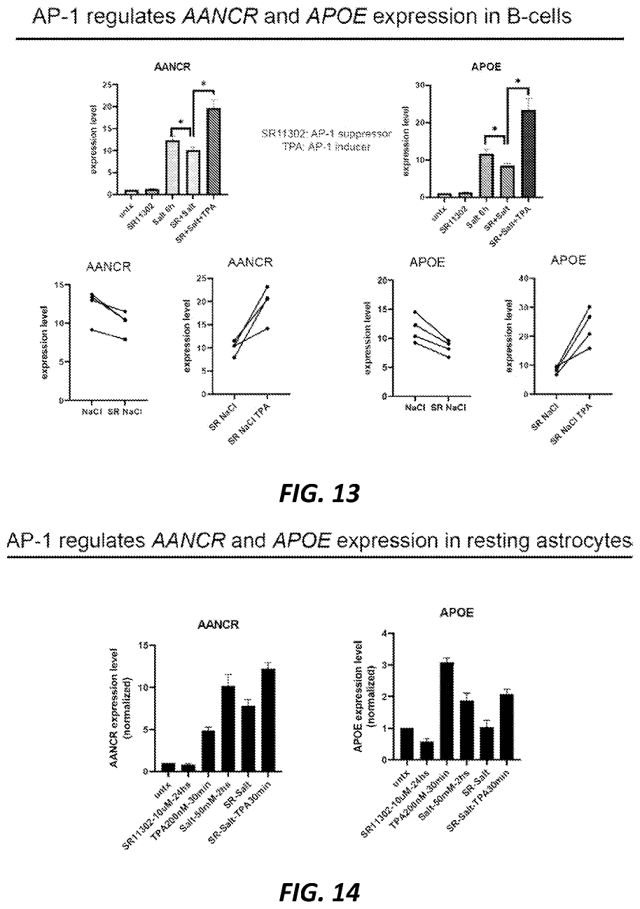

Stress leads to more AANCR expression. In the case of osmotic stress, others showed that signaling downstream of the DNA damage response protein ATM induces the stress response (FIG. 10). Cells from individuals with mutations in ATM were observed to see if they have a different response to osmotic stress.

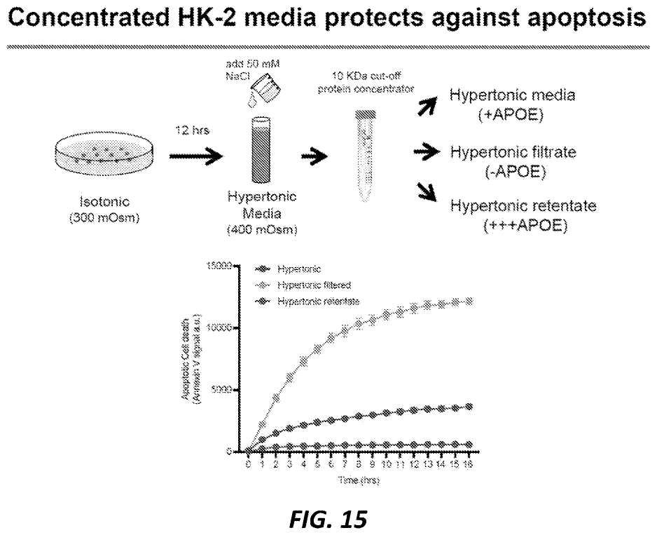

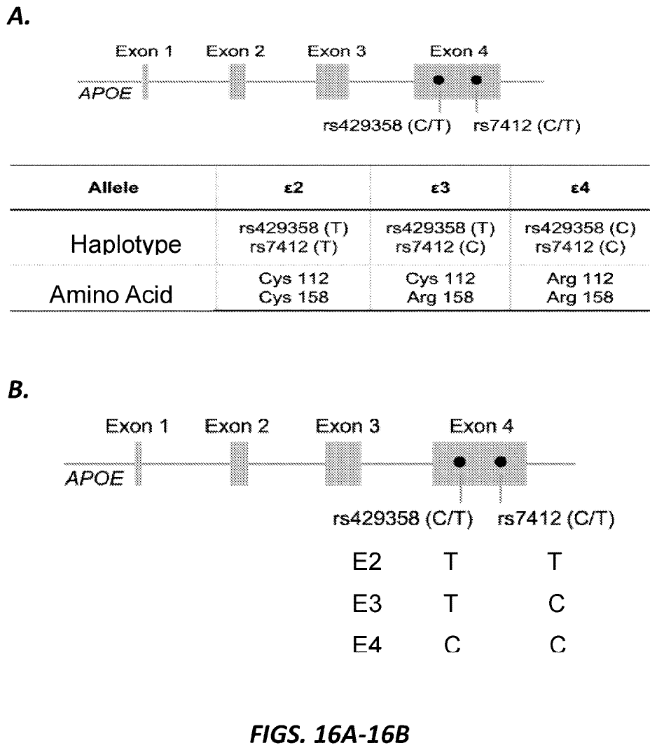

In cells with two normal copies of ATM there is increased expression of the tonicity-responsive transcription factor NFAT5 and AANCR and APOE. But in cells with one (AT het) or two (AT pt) copies of mutant ATM, there is no induction of NFAT5, AANCR, or APOE. Thus, ATM is an upstream regulator of AANCR (FIG. 11). NFAT5 does not bind AANCR. But MAP kinase 8 is downstream of ATM and activates JUN and FOS. JUN-FOS heterodimers form the transcription factor AP-1. AP-1 may bind and activate AANCR (FIG. 12). ENCODE consortium did chromatin immunoprecipitation in HEPG2 cells, which express AANCR and APOE. Data show that AP-1 subunits FOSL2 and JUN bind AANCR.

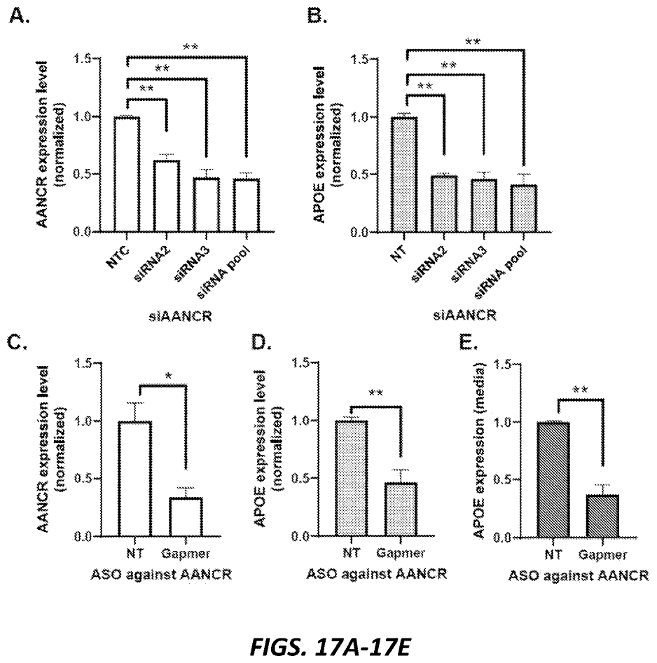

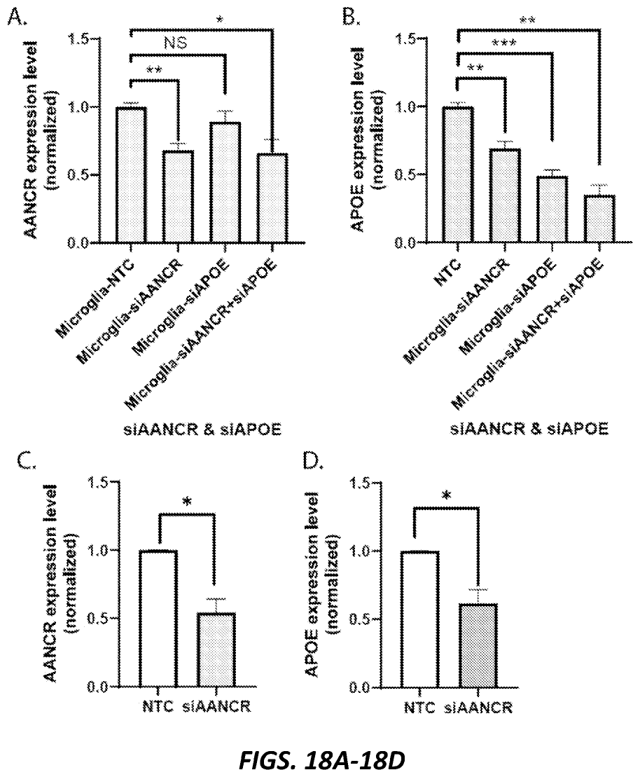

Treating B-cells with AP-1 inhibitor (SR11302) represses both AANCR and APOE expression (FIG. 13). Treating with an AP-1 activator (TPA) induces AANCR and APOE (FIG. 13). Treating cells with hypertonic salt and the AP-1 inhibitor represses the stress responsive gene expression, which can be rescued with the AP-1 activator, TPA (FIG. 13). Treating astrocytes with AP-1 inhibitor (SR11302) represses both AANCR and APOE expression (FIG. 14). Treating with an AP-1 activator (TPA) induces AANCR and APOE (FIG. 14). Treating cells with hypertonic salt and the AP-1 inhibitor represses the stress responsive gene expression, whereas hypertonic salt with the AP-1 activator leads to greater induction than hypertonic salt alone (FIG. 14). Thus, using small molecules that activate or inhibit AP-1 result in higher or lower AANCR expression in different cell types. Together, the results show that AANCR is regulated by ATM-MAPK-AP1 (JUN/FOS) regulatory pathway.

As a treatment, AANCR expression can be fine-tuned—with, for example, small molecules or antisense oligos (allele-specific oligonucleotide (ASO)) can be delivered to cells, including specific cell types (such as a delivery vehicle with anti-AANCR and optionally anti-APOE4 as a payload; for example, lipid nanoparticles with cell type specific receptor ligands). This treatment can be used for Alzheimer's disease, traumatic brain injury, osmotic demyelination syndrome, or even kidney disease (FSGS, Focal Segmental glomerulosclerosis).

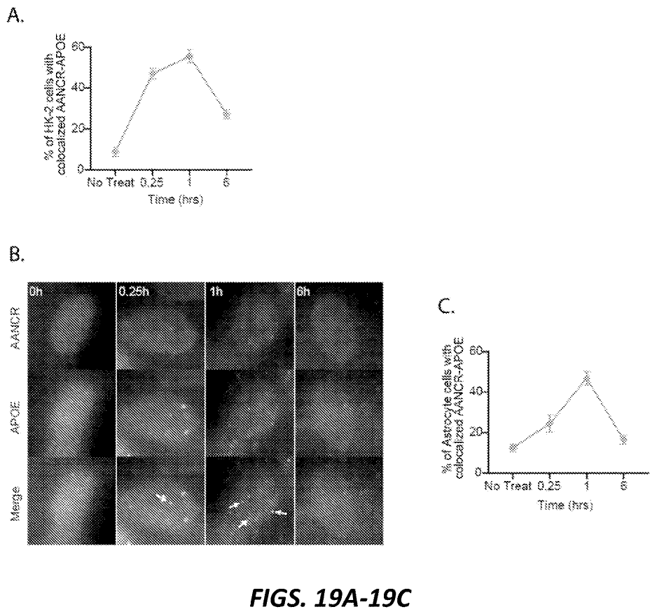

Media from cells was collected and protein concentrator was used to create a fraction without APOE or a fraction with high concentration of APOE. Compared to regular media, there was more apoptosis in cells in hypertonic media without APOE (FIG. 15; orange line). Whereas cells exposed to hypertonic media with concentrated APOE (blue line) are protected against cell death (FIG. 15). This suggests increased APOE can be protective in conditions of osmotic cell stress and may be beneficial in conditions like osmotic demyelination syndrome.

Additional experiments demonstrate that stress responsive AANCR-APOE regulation is conserved in mouse.

### Example III—Enhancer RNA, AANCR, Regulates APOE Expression in the Central Nervous System

### Introduction

Most of the human genome encodes noncoding RNA. Research by the ENCODE consortium revealed that approximately 80% of the human genome undergoes transcription, but less than 2% codes for proteins (1). Thus, a significant portion of the genome encodes various noncoding RNA. Among these, a group is enhancer RNAs (2, 3) that play roles in regulating gene expression.

Another feature of human genomes is the sequence differences among individuals. The DNA sequences of unrelated individuals differ by about 0.1%. These sequence differences account for phenotypic differences and disease susceptibility. Some of these sequence differences modulate the penetrance and expression of diseases. These genetic modifiers alter the onset and/or severity of diseases by modifying the primary disease-causing genes (4-6). An example is the polymorphisms in TGFB1 that modulate the severity of lung disease in cystic fibrosis by influencing the expression of CFTR (7).

Given the large number of sequence variants and enhancer RNAs in human cells. It is to be expected that there are sequence variants of enhancer RNA that influence their enhancer functions. Genetics of gene expression studies (8, 9) have uncovered enhancer variants that explain variation in gene expression across individuals. For instance, the enhancer variants that influence MYC expression levels and consequently the risk of colorectal cancer (10).

Previously, an enhancer RNA that named APOE-activating noncoding RNA, AANCR was identified as a genetic modifier of Alzheimer's Disease that influences the expression of the primary disease-causing gene, APOE. It was shown that AANCR regulates APOE expression in various peripheral cell types (11). Sequence variants in AANCR are significantly associated with Alzheimer's Disease in various genome-wide association studies (11). AANCR and its regulation by RNA sequence and structure were identified while studying R-loops.

In hepatocytes, the full-length AANCR is synthesized, and it enhances the expression of APOE. However, in other cells like B-cells and fibroblasts, only a partial transcript of AANCR is synthesized. This is because RNA Polymerase II elongation is blocked by R-loops. These R-loops are kept stable by sequential modifications of the RNA in the R-loops where adenosine gets N6-methylated and then the m6A is cleaved resulting in abasic sites. When these cells are under stress, the nascent RNA of AANCR does not undergo these chemical modifications. As a result, the R-loops are transient, allowing RNA Polymerase II to synthesize the full-length AANCR. This in turn facilitates the expression of APOE in B-cells and other peripheral cells, which typically do not express APOE at baseline.

Research revealed the role of AANCR in peripheral cells. Provided in this example is the focus to the central nervous system given that APOE is a susceptibility gene for Alzheimer's Disease. It was discovered that AANCR influences APOE levels in astrocytes, microglia, and iPSC-derived neurons, both under normal conditions and during mitochondrial oxidative stress. Notably, an ATM-mediated mechanism was uncovered that activates AANCR and subsequently APOE expression in microglia under mitochondrial stress. Additionally, the experiments indicated that decreasing AANCR-APOE expression in astrocytes lessens their inflammatory characteristics but reduces their mitochondrial functionality. Therefore, this example establishes AANCR as an enhancer RNA for APOE in both peripheral and central nervous system cells. It also sheds light on the specific signaling pathway that triggers AANCR-APOE during stress responses and identifies the downstream effects of this interaction.

### Materials and Methods

### Cell Culture

Human cultured B-cells were grown in Roswell Park Memorial Institute media, RPMI 1640, supplemented with 15% fetal bovine serum, 100 U/mL penicillin/100 g/mL streptomycin, and 2 mmol/L L-glutamine.

Human astrocytes were cultured in Astrocyte Medium following ScienCell's protocol (https://sciencellonline.com/PS/1800.pdf).

Human microglia were grown in Dulbecco's Modified Eagle Medium (DMEM) supplemented with 10% fetal bovine serum, 100 U/mL penicillin and 100 g/mL streptomycin.

HEK 293T cells (ATCC) were maintained in DMEM medium with 10% FBS, 1% GlutaMAX(Gibco, 35050061), and 1% P/S in a humidified chamber with 5% C02.

All cells were grown at 37° C., 5% C02. Adherent cells were passaged every 72 hours using trypsin-EDTA 0.025% for astrocytes and 0.05% for the other cell lines.

siRNA Knockdown of AANCR and APOE in Astrocytes, Microglia, and iPSC-Neurons

Cells were seeded in 6-well plates or T-25 flasks. Cells grew to 50-60% confluence and were transfected with siRNA using Lipofectamine RNAiMax to a final concentration of 10 nM. Cells were harvested for expression or for protein analysis 24- and 48-hours post-transfection. The siRNA targeting AANCR and APOE are listed in Resource table.

### ASO Against AANCR in Astrocytes and Microglia:

Cells were grown to a confluence of 50% in T-25 flasks or 6-well plates. The AANCR gapmer was transfected using Lipofectamine 3000 at a working concentration of 10 nM to astrocytes or microglia. Cells were harvested four days post-transfection and gene expression was determined by SYBR Green PCR Master Mix on Applied Biosystem QuantStudio 5 Real-Time PCR System.

### Aβ Aggregates in Microglia, B-Cells, and Astrocytes

For the preparation of amyloid beta protein fragment 1-42 (Ab42) or amyloid beta protein fragment 1-40 (Ab40): Briefly, Ab42 or Ab40 was dissolved in dimethyl sulfoxide (DMSO) to a concentration of 5 mM, and then diluted to make 100 μM stocks which were incubated for 5 days at 37° C. Ab42 and Ab40 treatments: the cells were washed twice with PBS, and placed in serum-free media. Twenty-four hours later, Ab42 or Ab40 were added to the cells at a final concentration of 10 μM in the media, and cells were treated for 24, 48, 54, and 72 hours.

### Antimycin a in Microglia, Astrocytes and B-Cells

Antimycin A was added to the media of the growing cells with doses 0-10 μM for 0 to 24 hours.

### Paraquat in Astrocytes

Paraquat was used to treat astrocytes with doses of 10, 25, 50, and 100 μM for 24 hours. SR11302 was used to treat astrocytes using doses of 2 and 5 uM for 24 hours.

SR11302 and TPA in Astrocytes with/without Hypertonic Stress

The cells were cultured in their growth media supplemented with 50 mM NaCl to a final 400 mOsm for 2 hours. SR11302 was added at 2 uM or 5 uM for 24 hrs followed by addition of TPA at 10 ng/mL or 50 ng/mL for 30 min and 2 hrs to check APOE and AANCR expression in B-cells and astrocytes.

### Jun Inhibitor SP600125

The Jun inhibitor SP600125 was either used at 20 uM for 16 hrs only, or SP600125 at 20 uM was used for 16 hrs followed by addition of antimycin-A at 10 uM for 2 hrs and 6 hrs to check APOE and AANCR expression in microglia.

qPCR of AANCR and APOE

Primers of AANCR and APOE for qPCR were designed as Resource table shows. Total RNA was isolated using the RNeasy Mini-Kit and 0.5 g RNA was converted to cDNA using Taqman RT reagents kit using random hexamer priming. Gene expression was determined by SYBR Green PCR Master Mix on Applied Biosystem QuantStudio 5 Real-Time PCR System.

### APOE Quantitation in Media

APOE quantitation in media: After serum starvation for 24 hours in non-FBS media, the media from each sample was collected and concentrated ten times with Protein Concentrator PES, 10K MWCO. After addition of 2× sample loading buffer and running the gels, western blots were visualized using APOE antibody. Also, APOE ELISA was performed using the Human APOE/Apolipoprotein E ELISA Kit.

### Western Blots

Western blots of phospho-ATM, total ATM, phospho-ERK1/2, total ERK1/2, phospho-c-JUN, total c-JUN: Following treatment of cells with the reagents mentioned above, they were washed with ice-cold PBS and placed in RIPA buffer supplemented with Protease Inhibitor Cocktail, Phosphatase Inhibitor Cocktail 2 and Phosphatase Inhibitor Cocktail 3, and held on ice 10 minutes. Western blots were performed by running cell lysates on 4-12% Bis-Tris Protein Gels in 1×MES SDS running buffer. Proteins were transferred to PVDF membrane in 1× transfer buffer at 100V for 1 hour at 4° C. The membranes were blocked with 5% powdered milk in 1×TBST buffer. Primary antibodies are shown in the table below.

RNA (cDNA)-seq

From astrocytes treated with siRNA against ANNCR or/and APOE or non-target siRNA, sequencing libraries were prepared using TruSeq Stranded Total RNA Library Prep Kit (Illumina). Sequencing was performed on NovaX and >150 million 100-nt reads were generated from each sample. Sequencing reads were aligned to the GRCh38 (hg38) human reference sequence using GSNAP (v. 2021-12-17) (Wu and Nacu, 2010) with one mismatch allowed, and soft clipping on. Expression was quantified using Cufflinks v.2.2.1 (Trapnell et al 2012).

### CRISPR-Editing

sgRNA sequence to target the AP-1 site at AANCR locus was selected using CRISPR Targets tool in UCSC genome browser (genome.ucsc.edu). sgRNA oligo was cloned into plasmid pSpCas9(BB)-2A-GFP (PX458), a gift from Feng Zhang (Addgene plasmid #48138; n2t.net/addgene:48138; RRID:Addgene_48138) (ref PubMed 24157548). The 100-nucleotide single-stranded DNA oligonucleotide (sODN) repair template (see resource table) was centered at the predicted CRISPR/Cas9 cleavage site and designed introduce three-point mutations in the AP-1 binding site and prevent recutting after gap repair. pSpCas9(BB)-2A-GFP with the sgRNA targeting AANCR were co-transfected with the sODN into HEK 293T. Cells were plated at 2.5×105 per well in 12-well dishes and the transfected with 500ng pSpCas9(BB)-sgRNA-2A-GFP and 1 μl of 10 μM ssODN template DNA using Lipofectamine LTX (Invitrogen, 15338500). 36-48 hours after transfection, cells were well disassociated and GFP positive cells were FACS sorted into 96-well plates (one cell/well) pre-coated with gelatin for 2-3 weeks to allow for single colony growth. Following cell expansion genomic DNA was isolated from single colonies and editing was verified by Sanger sequencing.

### Extracellular Flux Analysis

For Seahorse analysis (XFe24, Agilent Technologies), astrocytes were seeded at a density of 100,000 cells/well in poly-L-lysine-coated XFe24 cell culture microplates. Cells were transfected with siRNA against AANCR as described above, and after 48 hours the astrocytes were washed twice with XF DMEM medium (Agilent Technologies) supplemented with 100 μM pyruvate, 200 μM L-glutamine, and 1.1 μM glucose, and 0.5% serum (ScienCell), then allowed to incubate in supplemented XF DMEM for 1 hour at 37° C. and 0% CO2. Cells were then tested for oxygen consumption rate (OCR) and extracellular acidification (ECAR) over a continuous 12-hour period with measurements taken every 30 minutes. ECAR and OCR values were then normalized to cell counts in each well.

### Chromatin Immunoprecipitation

Two independent CRSIPR edited cells lines where the AP-1 site was mutated and two cell lines where the AP-1 site was not edited were used for ChTP following the protocol described in (Watts et al NAR) using 5 g antibody against Jun (Cell Signaling Cat #9165S) or IgG (Millipore Cat #12-370). Factor occupancy was determined by qPCR using primers listed in the resource table.

smFISH

FISH probes to AANCR (26 probes) and APOE (29 probes) were designed with Stellaris Probe Designer (biosearchtech.com/stellaris-designer) and ordered from Biosearch Stellaris with Quasar 570 (AANCR probes) or Quasar 670 (APOE) label. Probes were hybridized as previously described (Rodriguez et al., Cell, 2019; PMID: 30554876). Briefly cells were fixed using 4% PFA for 10 min at room temperature. Excess PFA was removed by washing with PBS and then permeabilized with 70% ethanol overnight at 4° C. Probe hybridization was carried out at 37° C. following the adherent mammalian cell protocol from Biosearch Stellaris. Images were acquired and 900 to 1300 nuclei per sample were analyzed as previously described (Hoffman et al., Mol Cell. 2022; PMID: 3507770).

| | | |
|---|---|---|
| REAGENT or RESOURCE | SOURCE | IDENTIFIER |

Antibodies

| | | |
|---|---|---|
| phospho-ATM | Abcam | [symbol] |
| Total ATM | Abcam | [symbol] |
| phospho-ERK1/2 | Cell Signaling | [symbol] |
| Total ERK1/2 | R&D | [symbol] |
| Phospho-C-JUN | Cell Signaling | [symbol] |
| Total C-JUN | Invitrogen | [symbol] |
| APOE | Sigma | [symbol] |
| APOE | Santa Cruz | [symbol] |
| GAPDH | Protein Tech | [symbol] |

Chemicals

| | | |
|---|---|---|
| [symbol] | [symbol] | [symbol] |
| Roswell Park Memorial Institute media | Thermofisher | [symbol] |
| (RPMI 1640) | | |
| Dulbecco's Modified Eagle Medium (DMEM) | Thermofisher | [symbol] |
| Astrocyte Medium | ScienCell | [symbol] |
| fetal bovine serum | Biowest | [symbol] |
| penicillin/streptomycin | Thermofisher | [symbol] |
| L-glutamine | Thermofisher | [symbol] |
| trypsin-EDTA | Thermofisher | [symbol] |
| Lipofectamine RNAiMax | Thermofisher | [symbol] |
| Lipofectamine 3000 | Thermofisher | [symbol] |
| SYBR Green PCR Master Mix | Thermofisher | [symbol] |
| amyloid beta protein fragment 1-40 | MilliporeSigma | [symbol] |
| amyloid beta protein fragment 1-42 | MilliporeSigma | [symbol] |
| dimethyl sulfoxide | MilliporeSigma | [symbol] |
| Antimycin A | MilliporeSigma | [symbol] |
| Paraquat | MilliporeSigma | [symbol] |
| SR11302 | Tocris Bioscience | [symbol] |
| TPA (12-O-Tetradecanoylphorbol-13-Acetate) | Cell Signaling | [symbol] |
| Jun inhibitor SP600125 | MilliporeSigma | [symbol] |
| Protease Inhibitor Cocktail | MilliporeSigma | [symbol] |
| Phosphatase Inhibitor Cocktail 2 | MilliporeSigma | [symbol] |
| Phosphatase Inhibitor Cocktail 3 | MilliporeSigma | [symbol] |
| 4-12% Bis-Tris Protein Gels | Thermofisher | [symbol] |
| MES SDS running buffer | Thermofisher | [symbol] |
| Transfer buffer | Thermofisher | [symbol] |
| Powdered milk | Thermofisher | [symbol] |
| Tris Buffered Saline, with Tween 20 (TBST) | MilliporeSigma | [symbol] |
| TRIzol | Thermo Fisher Scientific | [symbol] |
| TRIzol LS | Thermo Fisher Scientific | [symbol] |
| Biotin-11-ATP | Perkin-Elmer | [symbol] |
| Biotin-11-UTP | Perkin-Elmer | [symbol] |
| Biotin-11-CTP | Pedon-Elmer | [symbol] |
| Biotin-11-GTP | Perkin-Elmer | [symbol] |
| 3M sodium acetate pH 5.5 | Thermo Fisher Scientific | AM9740 |
| Dynabeads M-280 Streptavidin | Thermo Fisher Scientific | 11205D |
| T4 Polynucleotide Kinase | NEB | M0201S |
| RNA 5′ Pyrophosphohydrolase (RppH) | NEB | M0356S |
| [symbol] | Thermo Fisher Scientific | AM2694 |
| SuperScript III Reverse Transcriptase | Thermo Fisher Scientific | 18080-044 |
| Phusion High-Fidelity DNA Polymerase | NEB | M0530S |
| T4 RNA ligase I | NEB | M0204S |
| 20% N-Lauroylsarcosine sodium salt | Sigma-Aldrich | L7414-10ML |
| solution [Sarkosyl] | | |
| Betaine [5M] | Sigma-Aldrich | B0300-1VL |
| Igepal CA-630 | Sigma-Aldrich | I3021-100ML |

Critical commercial Kits and divice

| | | |
|---|---|---|
| RNeasy Mini-Kit | Qiagen | Cat#74104 |
| Taqmen RT reagents kit | Thermofisher | Cat#N8080234 |
| Human APOE/Apolipoprotein E ELISA Kit | MilliporeSigma | Cat#RAB0613-1KT |
| Protein Concentrator PES, 10K MWCO | (ThermoScientific | Cat#88517 |

Experimental models: Cell lines

| | | |
|---|---|---|
| Human cultured B-cells | Coriell Institute | many and will be |
| | | filled |
| Human astrocytes | ScienCell | Cat#1800-5 |
| Human microglia | ATCC | [symbol] |

Primers for qPCR

| primer name | sequence |
|---|---|
| 5′AANCR-F | AGTCATTTGCCCAAGGTCAC |
| 5′AANCR-R | TCCCCACATGTGAAATGAGA |
| APOE-F | CAGGTCACCCAGGAACTGAG |
| APOE-R | TCCAGTTCCGATTTGTAGGC |

siRNA for qPCR

| Name | Sense Sequence | Antisense Sequence |
|---|---|---|
| siAANCR1 | CCAUGCUCAAUACACGUUAtt | UAACGUGUAUUGAGCAUGGag |
| siAANCR2 | GGAUUUAUAACAGGGCUUAtt | UAAGCCCUGUUAUAAAUCCca |
| siAANCR3 | UCCUAACCUUAACCCAGAAtt | UUCUGGGUUAAGGUUAGGAtt |
| siAANCR_AP1 | CAUCUCUGCACUCAGAGACAGtt | CTGTCTCTGAGTGCAGAGATGtt |
| siAPOE1 | GGAGUUGAAGGCCUACAAAtt | UUUGUAGGCCUUCAACUUu |
| siAPOE2 | GACAAUCACUGAACGCCGAtt | UCGGCGUUCAGUGAUUGUCgc |
| siAPOE3 | CUAGUUUAAUAAAGAUUCAtt | UGAAUCUUUAUUAAACUAGgg |
| siAPOE4 | AAGCGGUGGAGACAGAGCCtt | GGCUCUGUCUCCACCGCUUtt |

[symbol]

indicates data missing or illegible when filed

### (SEQ ID NOS: 26-45)

### Results

AANCR, an enhancer RNA, regulates APOE expression in astrocytes, microglia and neurons.

In the brain, APOE is synthesized mostly by astrocytes, but the transcriptional regulation of APOE by astrocytes was unknown. In the previous study, it was found that the enhancer RNA, APOE-activating noncoding RNA, AANCR, regulates the expression of APOE in peripheral tissues (11). Specifically, AANCR regulates the expression of APOE in hepatocytes, and induces APOE synthesis in B-cells, fibroblasts, and renal tubule cells in response to stress (11). Here, it was asked if AANCR also regulates APOE expression in the central nervous system.

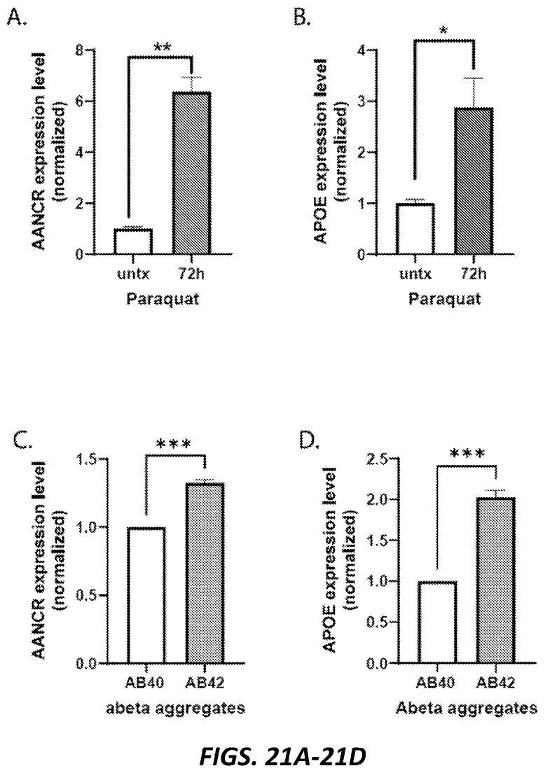

A genetic approach was undertaken by asking if altering AANCR expression would result in changes in APOE expression. Using RNA interference in astrocytes, AANCR was knocked down with different siRNAs individually and as a pool. The results showed that AANCR expression level was reduced about 50% by RNA interference (FIG. 17A, P<0.005); in the same cells, APOE expression also decreased significantly by about 50% (FIG. 17B, P<0.005). To ensure the findings are specific, AANCR was also knocked down with a single-stranded DNA oligonucleotide specific to AANCR. The results showed that the antisense oligonucleotides (ASO) decreased AANCR expression by more than 50% (P<0.05, FIG. 17C), and correspondingly APOE expression was significantly reduced (P<0.005, FIG. 17D). Given that APOE is a secreted protein, the amount of APOE protein in the media was measured. FIG. 17E showed that the knockdown of AANCR also decreased the amount of secreted APOE protein. Astrocytes with lower AANCR expression secreted about 40% less APOE proteins (FIG. 17E, P<0.005). Together these results show that the enhancer RNA, AANCR, influences the expression of APOE in astrocytes.

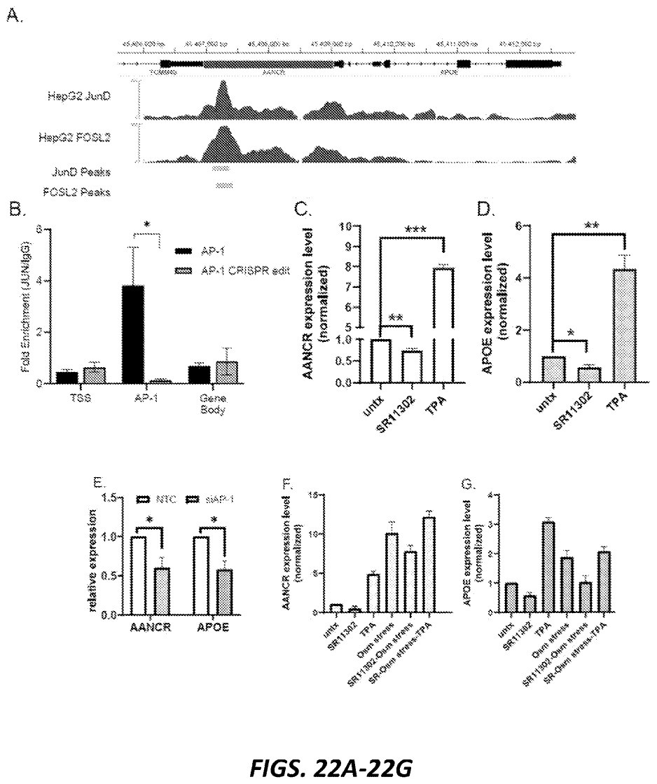

Like astrocytes, microglia express APOE but at a lower level. It was asked if, in microglia, APOE expression is also regulated by AANCR. AANCR was knocked down and it was found that APOE level decreased correspondingly (FIGS. 18A & 18B, P<0.005). APOE was also knocked down by RNA interference, and while the APOE expression decreased (P<0.005, FIG. 18B), AANCR expression did not change (FIG. 18A). AANCR is a noncoding RNA found upstream of APOE, this result shows that AANCR is an independent transcript that regulates APOE, and there is no apparent feedback loop where APOE regulates AANCR. It was also confirmed that AANCR knockdown significantly reduced APOE expression in iPSC-derived neurons (FIGS. 18C & 18D).

### AANCR Regulates APOE by Physical Interactions.

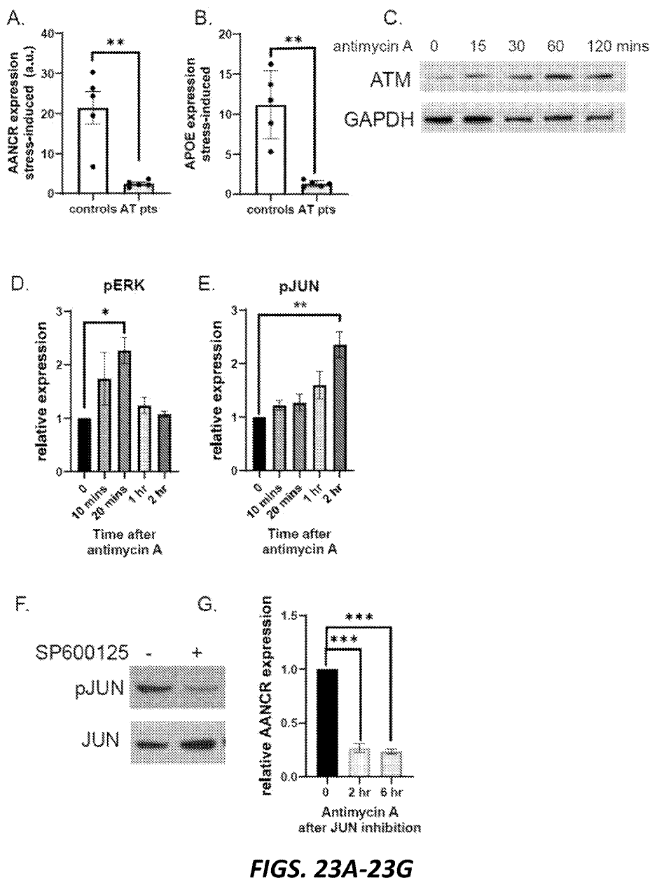

The results of the AANCR knockdown on APOE gene expression can be an indirect effect even though it is unlikely given that previously by Hi-C, it was found that AANCR interacts with APOE (11). To further examine if APOE is a direct target of AANCR, single-molecule fluorescence hybridization (smFISH) to measure the association between AANCR and APOE. Previously, it was found that APOE is induced in renal tubule cells following hypertonic stress (11). Here, salt (50 mM) was added to renal tubule cells, and within 15 minutes, in about 40% of the nuclei (P<0.001), AANCR co-localized with APOE. FIG. 19A showed a time-dependent increase in the number of nuclei where AANCR and APOE co-localized. This experiment was then repeated in astrocytes. At baseline, AANCR is co-localized with APOE in more than 15% of the nuclei, and within 15 minutes of exposure to hypertonic stress, there was a significant (P<0.03) increase in the colocalization of AANCR and APOE transcripts. By 1 hour after stress exposure, AANCR and APOE were colocalized in more than 50% of the nuclei (FIGS. 19B and 19C). These results confirm that AANCR regulates APOE expression at baseline and in stress response by physically interacting with the APOE gene.

### AANCR Induces APOE Expression in Response to Mitochondrial Stress.

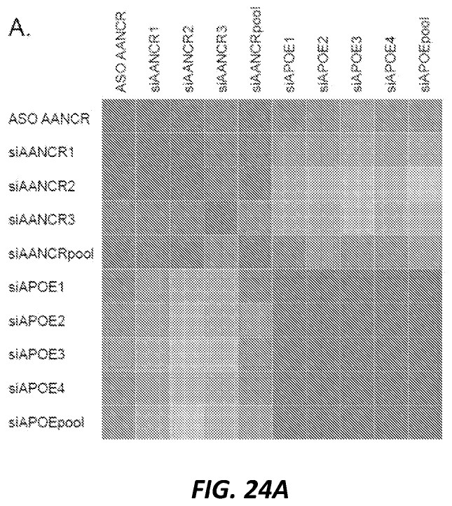

Next, AANCR's regulation of APOE in stress responses in the central nervous system was investigated. Oxidative stress that results from mitochondrial dysfunction is associated with neurodegenerative diseases including Alzheimer's Disease (12, 13). Since APOE is a major susceptibility gene of Alzheimer's Disease, it is important to understand its regulation in response to oxidative stress in the central nervous system. Oxidative stress was induced by inhibiting the mitochondrial electron transport chain. Microglia were treated with antimycin A which inhibits complex III of the mitochondrial electron transport chain (14, 15). FIG. 20A shows that antimycin A leads to a significant increase in AANCR expression and correspondingly a 50% and significant (P<0.01) increase in APOE expression level (FIG. 20B). The APOE is then secreted; FIG. 20C shows a time-dependent increase (>2-fold) of APOE protein abundance in the media.

It was then checked if AANCR-APOE expression is induced by other stressors, such as amyloid beta (A3) aggregates. A3 is a proteolytic product of amyloid precursor protein. Gamma secretase cleaves the C-terminus of amyloid precursor protein leading to Aβ42 (42 residues) and Aβ40 (40 residues). Aβ42 with two extra hydrophobic amino acids forms fibrils more efficiently, making it a major component of amyloid plaques. The accumulation of Aβ42 leads to oxidative stress (16-21). Microglia were incubated with Aβ40 or Aβ42 aggregates. FIG. 20D shows that amyloid beta aggregates with 42 amino acid residues significantly (P<0.001) increased AANCR expression. Consequently, APOE expression also increased (P<0.05, FIG. 20E). This experiment was repeated in human B-cells. The results showed that as in microglia, Aβ42 significantly increased AANCR and APOE expression in B-cells (FIGS. 20F and 20G). Thus, the expression response of AANCR and APOE to stress is similar in the cells in the CNS and the periphery.

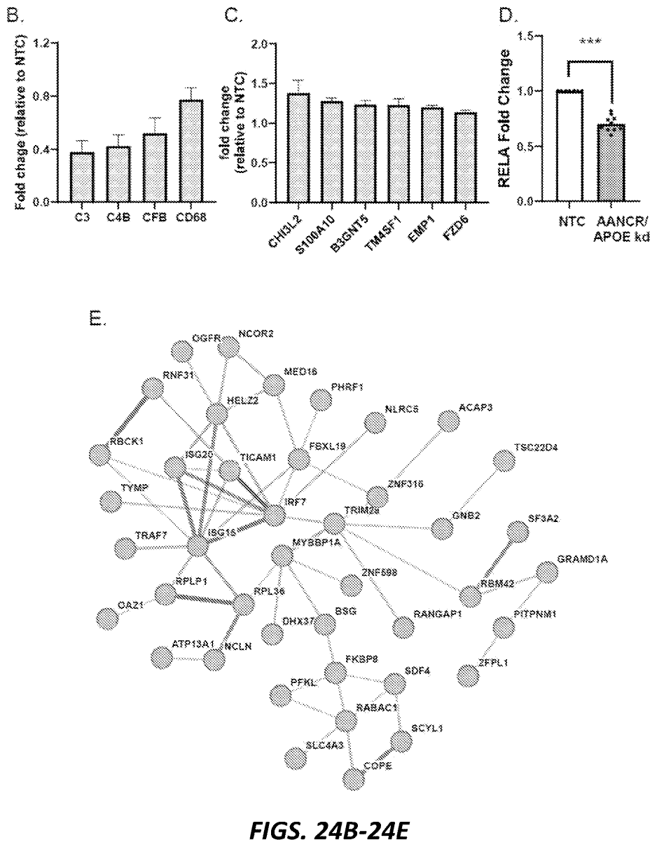

The study was extended further into astrocytes to assess if another mitochondrial stressor, paraquat, also induces AANCR expression. Paraquat induces oxidative stress through increased free radical production. Results showed that paraquat significantly induced AANCR (P<0.01) and APOE expression (P<0.05) in astrocytes (FIGS. 21A and 21B). Similarly, A β42 induces AANCR and APOE expression in astrocytes (FIGS. 21C and 21D).

Together, it was found that in human microglia, astrocytes and B-cells, various stresses induce AANCR and APOE expression further showing that AANCR regulates APOE expression in the periphery and the central nervous system at baseline and in response to stress. Signaling pathway that induces AANCR in response to stress.

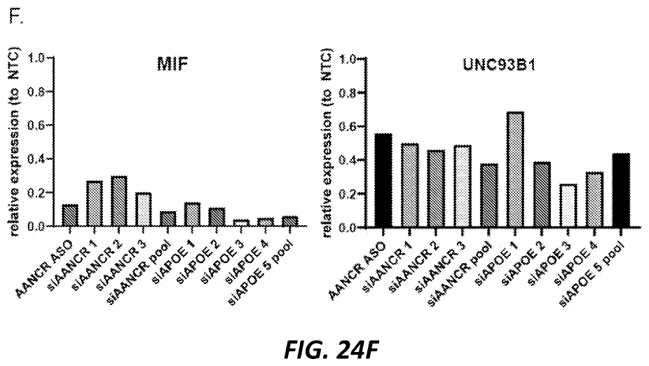

Next, it was asked how oxidative stress induces AANCR expression. Data from the ENCODE project identifying functional elements in the human genome show that JUN and FOS transcription factors constituting the activator-protein 1, AP-1, (22-25) bind to AANCR in HepG2 cells (FIG. 22A). Sequence analysis shows a putative AP-1 binding site underlying the peak identified by ENCODE (FIG. 22B). ENCODE data was followed up by assessing if the AP-1 binding results in the regulation of AANCR. Chromatin immunoprecipitation shows that JUN binds to the AP-1 site but the binding was abrogated when the sequence was mutated with CRISPR editing (FIG. 22B).

To ask if the AP-1 site in AANCR indeed regulates its expression in the CNS, astrocytes were treated with SR11302, a retinoid that specifically inhibits AP-1(26). The results showed that upon inhibition of AP-1, AANCR expression decreased significantly (P<0.01; FIG. 22C), and consequently APOE expression was reduced (P<0.05, FIG. 22D). The opposite experiment was then performed and treated the astrocytes with 12-0-tetradecanoyl-phorbol-13-acetate.

(TPA), an activator of AP-1 (27), and measured the AANCR expression level. The results showed that TPA induced the expression of AANCR by almost 8-fold (P<0.001; FIG. 22C) and the expression of APOE also increased correspondingly (P<0.01; FIG. 22D).

The assessment of the regulation of AANCR by AP-1 has so far depended on chemical perturbations of AP-1 with SR11302 and TPA. Since these molecules have a global effect siRNA specific to the AP-1 site in AANCR was designed and its effects assessed. In astrocytes, the knockdown of AANCR with siAP-1 led to a reduction in AANCR expression and a corresponding decrease in APOE expression (FIG. 22E). Together, these findings suggest that AP-1 regulates AANCR and therefore APOE expression at baseline and in response to stress.

To further examine if the AP-1 transcription factor complex regulates AANCR in stress response, osmotic stress was investigated since it was previously shown that osmotic stress induced the transcription of AANCR and APOE. AP-1 was inhibited in B-cells with SR11302 and exposed the cells to osmotic stress, the results showed that upon AP-1 inhibition, AANCR (FIG. S1A) and APOE (FIG. S1B) were induced at a significantly lower level in response to osmotic stress. The addition of TPA, an activator of AP-1, rescued the expression response of AANCR and APOE (FIGS. S1A and S1B) suggesting that in B-cells, AP-1 regulates AANCR transcription in stress response.

It was then asked if AP-1 also regulates AANCR response to osmotic stress in astrocytes. FIGS. 22F and 22G show that in astrocytes, SR11302 inhibition of AP-1 leads to significantly lower AANCR and APOE expression levels at baseline and in response to stress. The effect of AP-1 inhibition on AANCR expression is stronger in response to stress, consistent with the role of AP-1 in stress response (18, 19).

### ATM-ERK-AP1 Pathway Regulates AANCR

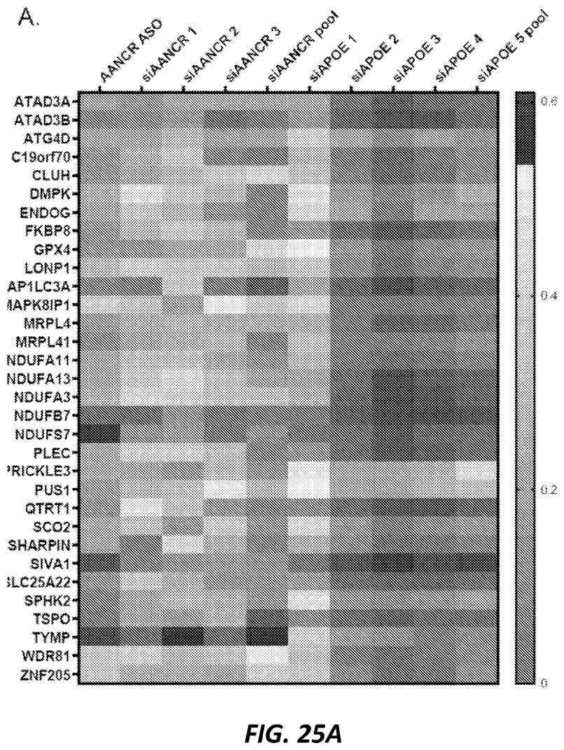

It was then sought to identify the upstream pathway that activates AP-1 to induce AANCR transcription. Burg and colleagues had shown that ATM is a signaling protein that mediates cellular response to osmotic stress (28). It was asked if ATM is a signaling factor that influences AANCR and therefore APOE expression in response to osmotic stress. Using B-cells from 5 patients with Ataxia Telangiectasia due ATM null mutations and B-cells from controls, the cells were exposed to osmotic stress and then AANCR and APOE expression was measured. FIG. 23A and FIG. 23B show that while AANCR and APOE expression were highly induced in the controls, they were only very modestly induced in the ATM null cells. Despite individual differences in AANCR and APOE stress-induced expression levels, the induction of AANCR (P<0.002) and APOE (P<0.008) expression levels are significantly higher in the control than the ATM null cells. This suggests that ATM is an upstream regulator of AANCR-APOE.

Next, it was asked if ATM regulation of AANCR-APOE is specific to peripheral B-cells and osmotic stress, or it is a general regulator of AANCR. To answer this question, the role of ATM in the induction of AANCR and APOE in microglia to mitochondrial stress was assessed. ATM activation can be assessed when microglia are exposed to mitochondrial stress. Microglia were treated with antimycin A as above (FIG. 20) and assessed for ATM-phosphorylation. FIG. 23C shows a representative immunblot with antibody against phospho-ATM. It was found that ATM is phosphorylated in microglia within 15 mins (P<0.01, FIG. 23C) of antimycin A treatment and the abundance of pATM continued to increase for 1 hour after antimycin. The results are consistent with earlier findings by Tanya Paull and colleagues, that ATM is involved in oxidative stress response (29). Here, Paull's salient results were extended and found that in microglia, oxidative stress also activates ATM which then induces AANCR-APOE expression.

Next, regulatory proteins downstream of ATM that led to the activation of AP-1 in AANCR were looked for. ERK, a kinase involved in oxidative stress responses including that induced by antimycin A (30-36), was looked at. Microglia were treated with antimycin A as above (FIG. 20), and then measured pERK1/2. Results showed that after antimycin A treatment, ERK was activated (P<0.05, FIG. 23D) about 20 mins, and after the activation of ATM. This finding is consistent with the knowledge that ERK is a target of ATM (37, 38).

Next, the phosphorylation of JUN was examined by treating microglia with antimycin A as above (FIG. 20). It was found that in the microglia, JUN was phosphorylated in a time-dependent fashion (FIG. 23E) to a maximum level between 1 and 2 hours (P<0.005), which is after the activation of ATM and ERK. To confirm that JUN phosphorylation activates AANCR, a small molecule SP600125 was used to inhibit JUN phosphorylation (39). In microglia, it was confirmed that SP600126 inhibited JUN phosphorylation but not JUN expression (FIG. 23F). The cells were then treated with SP600125 followed by antimycin A, and measured AANCR. The results showed that AANCR expression in response to oxidative stress is dependent on the activation or phosphorylation of JUN. FIG. 23G showed that in microglia where JUN cannot be activated, AANCR induction is significantly (P<0.001) reduced in response to antimycin A treatment.

Together the results elucidated the upstream signaling pathway that activates AANCR in response to mitochondrial stress. In response to stress, ATM is activated which then phosphorylates ERK then then phosphorylates JUN to activate the AP-1 site that promotes the transcription of AANCR, and therefore the expression of APOE.

Reducing the Expression of AANCR and/or APOE Shift Astrocytes to a Less Inflammatory State.

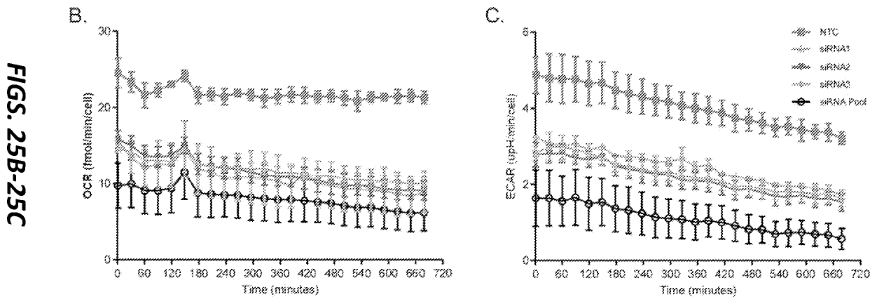

After finding that AANCR regulates APOE downstream of the ATM-ERK-AP1 pathway, the genes that are regulated by AANCR and APOE were examined. AANCR and APOE were silenced with siRNA or antisense oligonucleotides in astrocytes. AANCR and APOE were knocked down, each with 5 different siRNAs or ASO. In total, there were 10 gene silencing experiments plus nontarget controls. After validating that each siRNA and the ASO decreased AANCR and APOE expression significantly (FIG. S2), cDNA-sequencing was carried out with at least 100 million reads per sample. AANCR and APOE knockdown resulted in highly similar expression profiles. FIG. 24A is a correlation matrix; the diagonal is lined with comparisons of the same samples, the off-diagonal comparisons of the AANCR and APOE knockdown samples are highly similar to the self-comparisons. The similarity in expression profiles after AANCR and APOE knockdown provides further evidence that APOE is a major target gene of AANCR.

The cDNA-sequencing results showed that AANCR and APOE silencing significantly decreased APOE expression levels (P<2×10-8). Following AANCR and APOE knockdown, APOE expression decreased by an average of 77% and 84%, respectively. 597 additional genes are also differentially expressed (corrected P<0.05, at least 2-fold change in expression). Among these 598 genes are C3 and other genes (e.g., CFB, C4B) (FIG. 24B) in the complement pathway as well as chemokines (CXCL1, CXCL2, CXCL10) that characterize A1 astrocytes. The expression levels of these genes are significantly reduced in the astrocytes following AANCR and APOE knockdown. In contrast, the expression levels of genes such as EMP1 and S100A10 that characterize A2 astrocytes are increased (FIG. 24C). Thus, it appears that the knockdown of APOE leads to a dampening of the inflammatory features of astrocytes.

RELA, also known as p65, is a component of dimeric transcription factor NF-κβ that activates inflammatory cytokines (40-42). The expression level of RELA is significantly reduced (FIG. 24D) in the astrocytes with reduced APOE expression from both AANCR and APOE knockdown. The promoters of CXCL1 and CXCL2 contain NF-κβ binding sites, and their expression levels are regulated by RELA (43). The lower RELA expression likely contributes to the reduction in the chemokines (FIG. 24B) and the dampening of the inflammatory features in the astrocytes with reduced APOE expression.

Furthermore, network analysis of the 598 differentially expressed genes with k-means clustering (44) revealed a group of 115 genes that are significantly enriched (corrected P<10-10) for interaction with RIG1(45-49), a key receptor in the antiviral innate immune response. RIG1 detects RNA viruses, such as the coronavirus, and triggers the interferon response (50-54). Polymorphism in APOE has been implicated in influencing individual susceptibility to RNA viruses (55-57). FIG. 24E is a network of these genes based on the confidence model of network edges (44). The expression levels of these interferon-related genes such as ARMC5, IRF7, ISG15, ISG20 and DDX58 (RIG1) decreased significantly following AANCR and APOE knockdown. These interferon-related genes are involved in neuro-inflammation and found to be dysregulated in aging models and patients with early stages of Alzheimer's disease (58-62). These results further show reducing AANCR and therefore APOE expression may lessen neuroinflammation.

Additionally, almost 20% (110 genes, corrected P=5×10⁻⁶) of the 598 genes, interact with ESRβ. Estrogen receptor R plays a role in transcription regulation and neuroprotection against inflammation (63-65). ESRβ expression level in astrocytes decreased (by 31% siAANCR, 50% siAPOE, n=5 each) following AANCR and APOE knockdown, although it did not reach our significance cutoff. Among the genes in the ESRβ interactome are transcription regulators such as ARID5A, CACTIN, CC2D1A, CIC and SF3A2 that influence transcript stability, splicing and gene expression as well as genes such as DDX39, DHX37, MRPL4 and RPLP1 that regulate translation. This interactome also includes MIF, the migration inhibitory factor, and UNC93B1, a Toll-like receptor regulator. FIG. 24F shows following AANCR and APOE knockdown, the expression levels of MIF and UNC93B1 were reduced in astrocytes. MIF is cytokine that regulates immune response and is expressed by various cell types in the CNS and its overexpression has been implicated in inflammatory processes that worsen pathology in Alzheimer's Disease (66-70). Idubilast, an anti-inflammatory drug that decreases MIF has been proposed as a treatment for Alzheimer's Disease (71-73). UNC93B1 regulates immune responses by stabilizing and controlling the trafficking of Toll-like receptors such as TLR7 and TLR9 from the endoplasmic reticulum to endolysosomes and contributes to neuroinflammation (74-77). Together, these findings support AANCR-APOE's role in conferring inflammatory features of astrocytes.

### AANCR Knockdown and APOE Knockdown Decrease Mitochondrial Function in Astrocytes.

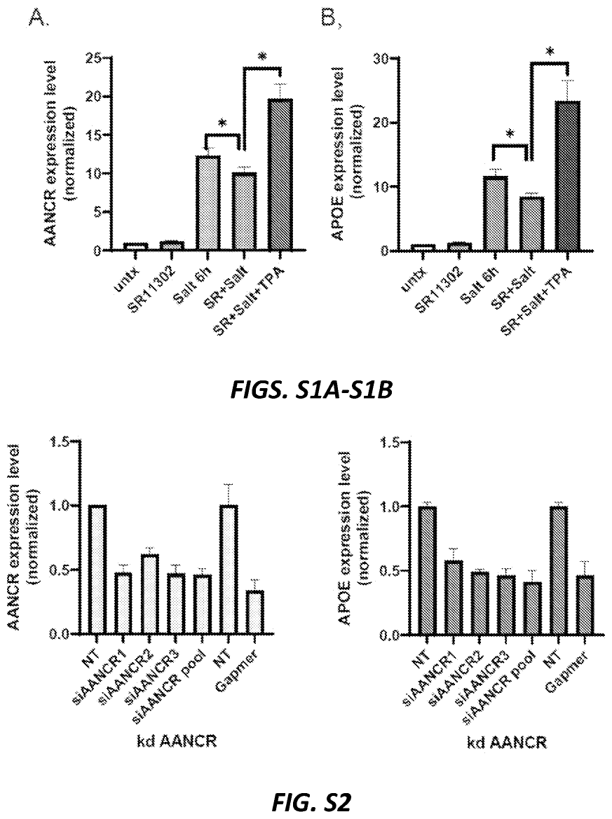

Among the differentially expressed genes following AANCR and APOE knockdown in astrocytes, a significant number of genes in the mitochondrial pathway (FDR=2×10⁻⁴) showed lower expression levels. These 32 mitochondrial-related genes include NDUFA3, NDUFB7 and NDUFS7 which encode proteins that constitute complex I of the electron transport chain. These genes are also part of the Mitocarta 3.0, an Inventory of Mammalian Mitochondrial Proteins and Pathways (78). FIG. 25A is a heatmap showing expression levels of these mitochondrial genes which are all reduced following AANCR and APOE knockdown suggesting APOE's role in mitochondrial function. This finding was followed up by assessing mitochondrial respiration and glycolysis (79, 80) in astrocytes following AANCR knockdown. Results show that the astrocytes have significantly (P<0.0001; FIG. 25B) lower oxygen consumption from impaired mitochondrial energy production. To ask if there are commensurate changes in glycolysis, the extracellular acidification rate (ECR) was measured, and it was found that a reduction in AANCR expression led to a significant (P<0.0001; FIG. 25C) decrease in glycolysis. Together the results show that AANCR regulates APOE expression which then facilitates mitochondrial function in astrocytes.

### Discussion

In this study, the knowledge of the regulatory role of APOE-activating non-coding RNA (AANCR) in the central nervous system was extended. Previously, AANCR was identified as an enhancer RNA of APOE in peripheral tissues and a genetic modifier of Alzheimer's Disease (AD), this work reveals that AANCR also modulates APOE expression in astrocytes, microglia, and neurons, both for cells at baseline and in response to stress. AANCR's contribution to AD susceptibility was investigated through its regulation of APOE in these cells in the central nervous system. The regulatory role of AANCR was demonstrated by gene knockdown in these cell types. Furthermore, the findings illustrate AANCR's role in upregulating APOE expression in response to stressors including antimycin A and Aβ42. To decipher the mechanism behind this induction, a binding site for the AP-1 transcription factor within AANCR was identified. It was found that stress conditions lead to microglial activation of ATM, which subsequently phosphorylates ERK. This cascade results in the activation of JUN, which binds to the AP-1 site of AANCR and stimulates its transcription, thereby enhancing APOE expression.

In exploring the functions of AANCR and APOE in astrocytes, siRNA mediated knockdown targeting each of these genes was employed. The analysis revealed strikingly similar expression profiles following the knockdown of AANCR and APOE. This similarity suggests that APOE is the principal gene regulated by AANCR, corroborated by the previous Hi-C and the current single-molecule fluorescent in situ hybridization data. A reduction in AANCR or APOE expression levels in astrocytes was accompanied by a decrease in the expression of C3 and genes associated with the complement pathways, as well as genes that encode cytokines. Conversely, there was an upregulation of EMP1 and S100A10 genes. These observations indicate that astrocytes with lower APOE expression, due to either AANCR or APOE knockdown, exhibit reduced inflammatory characteristics.

Previous research by Ben Barres and colleagues identified A1 astrocytes by their expression of C3 and other complement genes and showed the neurotoxicity of these cells. In contrast, A2 astrocytes, characterized by their expression of S100A10, are more neuroprotective (81, 82). The findings align with this characterization, suggesting that reduced APOE expression shifts astrocytes from an A1 to an A2 phenotype, a transition that could be advantageous in scenarios like acute CNS injury or infections where reducing inflammation is desired.

The discovery of genetic modifiers such as SMN2 in spinal muscular atrophy and BCL11A in sickle cell anemia and thalassemia, has paved the way for groundbreaking therapeutic interventions. For instance, Nusinersen, an RNA-based treatment for spinal muscular atrophy, enhances the inclusion of exon 7 in SMN2, leading to increased SMN protein production in patients (83). This advance has been instrumental in preventing fatalities and allowing patients to achieve developmental milestones (84, 85). Similarly, Exagamglogene autotemcel, a CRISPR-based therapy for sickle-cell anemia and β-thalassemia targets BCL11A to reactivate fetal hemoglobin production (86). Preliminary results are promising, showing a reduction or cessation of pain crises in sickle cell patients and eliminating the need for transfusions in β-thalassemia patients.

In this context, this discovery demonstrates that AANCR regulates APOE expression in the central nervous system. It was found that reducing AANCR expression leads to lower APOE levels, which appears to diminish the inflammatory characteristics of astrocytes.

### BIBLIOGRAPHY

1. ENCODE Project Consortium, Birney, E., Stamatoyannopoulos, J. A., Dutta, A., Guigó, R., Gingeras, T. R., Margulies, E. H., Weng, Z., Snyder, M., Dermitzakis, E. T., et al. (2007) Identification and analysis of functional elements in 1% of the human genome by the ENCODE pilot project. Nature, 447, 799-816.
2. De Santa, F., Barozzi, I., Mietton, F., Ghisletti, S., Polletti, S., Tusi, B. K., Muller, H., Ragoussis, J., Wei, C.-L. and Natoli, G. (2010) A large fraction of extragenic RNA pol II transcription sites overlap enhancers. PLoS Biol, 8, e1000384.
3. Kim, T.-K., Hemberg, M., Gray, J. M., Costa, A. M., Bear, D. M., Wu, J., Harmin, D. A., Laptewicz, M., Barbara-Haley, K., Kuersten, S., et al. (2010) Widespread transcription at neuronal activityregulated enhancers. Nature, 465, 182-187.
4. Nadeau, J. H. (2001) Modifier genes in mice and humans. Nat Rev Genet, 2, 165-174.
5. Rahit, K. M. T. H. and Tarailo-Graovac, M. (2020) Genetic Modifiers and Rare Mendelian Disease. Genes (Basel), 11, 239.
6. Genin, E., Feingold, J. and Clerget-Darpoux, F. (2008) Identifying modifier genes of monogenic disease: strategies and difficulties. Hum Genet, 124, 357-368.
7. Drumm Mitchell L., Konstan Michael W., Schluchter Mark D., Handler Allison, Pace Rhonda, Zou Fei, Zariwala Maimoona, Fargo David, Xu Airong, Dunn John M., et al. (2005) Genetic Modifiers of Lung Disease in Cystic Fibrosis. New England Journal of Medicine, 353, 1443-1453.
8. Morley, M., Molony, C. M., Weber, T. M., Devlin, J. L., Ewens, K. G., Spielman, R. S. and Cheung, V. G. (2004) Genetic analysis of genome-wide variation in human gene expression. Nature, 430, 743-747.
9. Cheung, V. G., Spielman, R. S., Ewens, K. G., Weber, T. M., Morley, M. andBurdick, J. T. (2005) Mapping determinants of human gene expression by regional and genome-wide association. Nature, 437, 1365-1369.
10. Pomerantz, M. M., Ahmadiyeh, N., Jia, L., Herman, P., Verzi, M. P., Doddapaneni, H., Beckwith, C. A., Chan, J. A., Hills, A., Davis, M., et al. (2009) The 8q24 cancer risk variant rs6983267 shows longrange interaction with MYC in colorectal cancer. Nat Genet, 41, 882-884.
11. Watts, J. A., Grunseich, C., Rodriguez, Y., Liu, Y., Li, D., Burdick, J. T., Bruzel, A., Crouch, R. J., Mahley, R. W., Wilson, S. H., et al. (2022) A common transcriptional mechanism involving R-loop and RNA abasic site regulates an enhancer RNA of APOE. Nucleic Acids Res, 50, 12497-12514.
12. Gonzilez-Reyes, R. E., Nava-Mesa, M. O., Vargas-Sinchez, K., Ariza-Salamanca, D. and Mora-Munoz, L. (2017) Involvement of Astrocytes in Alzheimer's Disease from a Neuroinflammatory and Oxidative Stress Perspective. Frontiers in Molecular Neuroscience, 10.
13. Onyango, I. G. and Khan, S. M. (2006) Oxidative Stress, Mitochondrial Dysfunction, and Stress Signaling in Alzheimer's Disease. Current Alzheimer Research, 3, 339-349.
14. Rieske, J. S., Baum, H., Stoner, C. D. and Lipton, S. H. (1967) On the Antimycin-sensitive Cleavage of Complex III of the Mitochondrial Respiratory Chain. Journal of Biological Chemistry, 242, 4854-4866.
15. Rieske, J. S. and Zaugg, W. S. (1962) The inhibition by antimycin A of the cleavage of one of the complexes of the respiratory chain. Biochem Biophys Res Commun, 8, 421-426.
16. Butterfield, D. A. (1997) beta-Amyloid-associated free radical oxidative stress and neurotoxicity: implications for Alzheimer's disease. Chem Res Toxicol, 10, 495-506.
17. Allan Butterfield, D., Castegna, A., Lauderback, C. M. and Drake, J. (2002) Evidence that amyloid betapeptide-induced lipid peroxidation and its sequelae in Alzheimer's disease brain contribute to neuronal death. Neurobiology of Aging, 23, 655-664.
18. Gu, L. and Guo, Z. (2013) Alzheimer's Aβ42 and Aβ40 peptides form interlaced amyloid fibrils. J Neurochem, 126, 305-311.
19. Cheignon, C., Tomas, M., Bonnefont-Rousselot, D., Faller, P., Hureau, C. and Collin, F. (2018) Oxidative stress and the amyloid beta peptide in Alzheimer's disease. Redox Biology, 14, 450-464.
20. Behl, C. (1997) Amyloid β-protein toxicity and oxidative stress in Alzheimer's disease. Cell Tissue Res, 290, 471-480.
21. Miranda, S., Opazo, C., Larrondo, L. F., Munoz, F. J., Ruiz, F., Leighton, F. and Inestrosa, N. C. (2000) The role of oxidative stress in the toxicity induced by amyloid β-peptide in Alzheimer's disease. Progress in Neurobiology, 62, 633-648.
22. Rauscher, F. J., Voulalas, P. J., Franza, B. R. and Curran, T. (1988) Fos and Jun bind cooperatively to the AP-1 site: reconstitution in vitro. Genes Dev, 2, 1687-1699.
23. Karin, M., Liu, Z. g and Zandi, E. (1997) AP-1 function and regulation. Curr Opin Cell Biol, 9, 240-246.
24. Vesely, P. W., Staber, P. B., Hoefler, G. and Kenner, L. (2009) Translational regulation mechanisms of AP-1 proteins. Mutation Research/Reviews in Mutation Research, 682, 7-12.
25. Shaulian, E. and Karin, M. (2001) AP-1 in cell proliferation and survival. Oncogene, 20, 2390-2400.
26. Huang, C., Ma, W.-Y., Dawson, M. I., Rincon, M., Flavell, R. A. and Dong, Z. (1997) Blocking activator protein-1 activity, but not activating retinoic acid response element, is required for the antitumor promotion effect of retinoicDacid. Proceedings of the National Academy of Sciences, 94, 5826-5830.
27. Lee, W., Mitchell, P. and Tjian, R. (1987) Purified transcription factor AP-1 interacts with TPAinducible enhancer elements. Cell, 49, 741-752.
28. Irarrazabal, C. E., Liu, J. C., Burg, M. B. and Ferraris, J. D. (2004) ATM, a DNA damage-inducible kinase, contributes to activation by high NaCl of the transcription factor TonEBP/OREBP. Proc Natl Acad Sci USA, 101, 8809-8814.
29. Guo, Z., Kozlov, S., Lavin, M. F., Person, M. D. and Paull, T. T. (2010) ATM activation by oxidative stress. Science, 330, 517-521.
30. Tai, P. and Ascoli, M. (2011) Reactive oxygen species (ROS) play a critical role in the cAMP-induced activation of Ras and the phosphorylation of ERK1/2 in Leydig cells. Mol Endocrinol, 25, 885-893.
31. Wu, H., Li, H., Wu, X., Zhao, J. and Guo, J. (2008) Reactive oxygen species mediate ERK activation through different Raf-1-dependent signaling pathways following cerebral ischemia. Neuroscience Letters, 432, 83-87.
32. Keshari, R. S., Verma, A., Barthwal, M. K. and Dikshit, M. (2013) Reactive oxygen species-induced activation of ERK and p38 MAPK mediates PMA-induced NETs release from human neutrophils. Journal of Cellular Biochemistry, 114, 532-540.
33. Gurjar, M. V., Deleon, J., Sharma, R. V. and Bhalla, R. C. (2001) Role of reactive oxygen species in IL-1β-stimulated sustained ERK activation and MMP-9 induction. American Journal of Physiology-Heart and Circulatory Physiology, 281, H2568-H2574.
34. El-Najjar, N., Chatila, M., Moukadem, H., Vuorela, H., Ocker, M., Gandesiri, M., Schneider-Stock, R. and Gali-Muhtasib, H. (2010) Reactive oxygen species mediate thymoquinone-induced apoptosis and activate ERK and JNK signaling. Apoptosis, 15, 183-195.
35. Monick, M. M., Powers, L. S., Barrett, C. W., Hinde, S., Ashare, A., Groskreutz, D. J., Nyunoya, T., Coleman, M., Spitz, D. R. and Hunninghake, G. W. (2008) Constitutive ERK MAPK Activity Regulates Macrophage ATP Production and Mitochondrial Integrity1. The Journal of Immunology, 180, 7485-7496.
36. Garg, T. K. and Chang, J. Y. (2003) Oxidative stress causes ERK phosphorylation and cell death in cultured retinal pigment epithelium: Prevention of cell death by AG126 and 15-deoxy-delta 12, 14-PGJ2. BMC Ophthalmol, 3, 5.
37. Khalil, A., Morgan, R. N., Adams, B. R., Golding, S. E., Dever, S. M., Rosenberg, E., Povirk, L. F. and Valerie, K. (2011) ATM-dependent ERK signaling via AKT in response to DNA double-strand breaks. Cell Cycle, 10, 481-491.
38. Tang, D., Wu, D., Hirao, A., Lahti, J. M., Liu, L., Mazza, B., Kidd, V. J., Mak, T. W. and Ingram, A. J. (2002) ERK activation mediates cell cycle arrest and apoptosis after DNA damage independently of p53. J Biol Chem, 277, 12710-12717.
39. Bennett, B. L., Sasaki, D. T., Murray, B. W., O'Leary, E. C., Sakata, S. T., Xu, W., Leisten, J. C., Motiwala, A., Pierce, S., Satoh, Y., et al. (2001) SP600125, an anthrapyrazolone inhibitor of Jun N-terminal kinase. Proceedings of the National Academy of Sciences, 98, 13681-13686.
40. Xia, Y., Pauza, M. E., Feng, L. and Lo, D. (1997) RelB regulation of chemokine expression modulates local inflammation. Am J Pathol, 151, 375-387.
41. Beg, A. A. and Baldwin, A. S. (1993) The I kappa B proteins: multifunctional regulators of Rel/NFkappa B transcription factors. Genes Dev, 7, 2064-2070.
42. Gilmore, T. D. (1999) The Rel/NF-κB signal transduction pathway: introduction. Oncogene, 18, 6842-6844.
43. Burke, S. J., Lu, D., Sparer, T. E., Masi, T., Goff, M. R., Karlstad, M. D. and Collier, J. J. (2014) NF-κB and STAT1 control CXCL1 and CXCL2 gene transcription. American Journal of Physiology—Endocrinology and Metabolism, 306, E131.
44. Szklarczyk, D., Kirsch, R., Koutrouli, M., Nastou, K., Mehryary, F., Hachilif, R., Gable, A. L., Fang, T., Doncheva, N. T., Pyysalo, S., et al. (2023) The STRING database in 2023: protein-protein association networks and functional enrichment analyses for any sequenced genome of interest. Nucleic Acids Res, 51, D638-D646.
45. Oughtred, R., Rust, J., Chang, C., Breitkreutz, B.-J., Stark, C., Willems, A., Boucher, L., Leung, G., Kolas, N., Zhang, F., et al. (2021) The BioGRID database: A comprehensive biomedical resource of curated protein, genetic, and chemical interactions. Protein Sci, 30, 187-200.
46. Wu, S.-F., Xia, L., Shi, X.-D., Dai, Y.-J., Zhang, W.-N., Zhao, J.-M., Zhang, W., Weng, X.-Q., Lu, J., Le, H.-Y., et al. (2020) RIG-I regulates myeloid differentiation by promoting TRIM25-mediated ISGylation. Proc Natl Acad Sci USA, 117, 14395-14404.
47. Inn, K.-S., Gack, M. U., Tokunaga, F., Shi, M., Wong, L.-Y., Iwai, K. and Jung, J. U. (2011) Linear ubiquitin assembly complex negatively regulates RIG-I- and TRIM25-mediated type I interferon induction. Mol Cell, 41, 354-365.
48. Zhao, C., Denison, C., Huibregtse, J. M., Gygi, S. and Krug, R. M. (2005) Human ISG15 conjugation targets both IFN-induced and constitutively expressed proteins functioning in diverse cellular pathways. Proc Natl Acad Sci USA, 102, 10200-10205.
49. Du, Y., Duan, T., Feng, Y., Liu, Q., Lin, M., Cui, J. and Wang, R.-F. (2018) LRRC25 inhibits type I IFN signaling by targeting ISG15-associated RIG-I for autophagic degradation. EMBO J, 37, 351-366.
50. Kawai, T. and Akira, S. (2008) Toll-like Receptor and RIG-1-like Receptor Signaling. Annals of the New York Academy of Sciences, 1143, 1-20.
51. Jiang, R., Ye, J., Zhu, B., Song, Y., Chen, H. and Cao, S. (2014) Roles of TLR3 and RIG-I in Mediating the Inflammatory Response in Mouse Microglia following Japanese Encephalitis Virus Infection. Journal of Immunology Research, 2014, e787023.
52. Li, J., Liu, Y. and Zhang, X. (2010) Murine Coronavirus Induces Type I Interferon in Oligodendrocytes through Recognition by RIG-I and MDA5. J Virol, 84, 6472-6482.
53. Takeuchi, O. and Akira, S. (2008) MDA5/RIG-I and virus recognition. Current Opinion in Immunology, 20, 17-22.
54. Hu, Y., Li, W., Gao, T., Cui, Y., Jin, Y., Li, P., Ma, Q., Liu, X. and Cao, C. (2017) The Severe Acute Respiratory Syndrome Coronavirus Nucleocapsid Inhibits Type I Interferon Production by Interfering with TRIM25-Mediated RIG-I Ubiquitination. Journal of Virology, 91, 10.1128/jvi.02143-16.
55. Wang, C., Zhang, M., Garcia, G., Tian, E., Cui, Q., Chen, X., Sun, G., Wang, J., Arumugaswami, V. and Shi, Y. (2021) ApoE-Isoform-Dependent SARS-CoV-2 Neurotropism and Cellular Response. Cell Stem Cell, 28, 331-342.e5.
56. Sheridan, D. A., Bridge, S. H., Felmlee, D. J., Crossey, M. M. E., Thomas, H. C., Taylor-Robinson, S. D., Toms, G. L., Neely, R. D. G. and Bassendine, M. F. (2012) Apolipoprotein-E and hepatitis C lipoviral particles in genotype 1 infection: Evidence for an association with interferon sensitivity. Journal of Hepatology, 57, 32-38.
57. Wong, M.-T. and Chen, S. S.-L. (2016) Emerging roles of interferon-stimulated genes in the innate immune response to hepatitis C virus infection. Cell Mol Immunol, 13, 11-35.
58. Roy, E. R., Wang, B., Wan, Y., Chiu, G., Cole, A., Yin, Z., Propson, N. E., Xu, Y., Jankowsky, J. L., Liu, Z., et al. (2020) Type I interferon response drives neuroinflammation and synapse loss in Alzheimer disease. J Clin Invest, 130, 1912-1930.
59. Taylor, J. M., Minter, M. R., Newman, A. G., Zhang, M., Adlard, P. A. and Crack, P. J. (2014) Type-1 interferon signaling mediates neuro-inflammatory events in models of Alzheimer's disease. Neurobiology of Aging, 35, 1012-1023.
60. Woodling, N. S., Wang, Q., Priyam, P. G., Larkin, P., Shi, J., Johansson, J. U., Zagol-Ikapitte, I., Boutaud, O. and Andreasson, K. I. (2014) Suppression of Alzheimer-Associated Inflammation by Microglial Prostaglandin-E2 EP4 Receptor Signaling. J. Neurosci., 34, 5882-5894.
61. Friedman, B. A., Srinivasan, K., Ayalon, G., Meilandt, W. J., Lin, H., Huntley, M. A., Cao, Y., Lee, S.-H., Haddick, P. C. G., Ngu, H., et al. (2018) Diverse Brain Myeloid Expression Profiles Reveal Distinct Microglial Activation States and Aspects of Alzheimer's Disease Not Evident in Mouse Models. Cell Rep, 22, 832-847.
62. de Rivero Vaccari, J. P., Brand, F. J., Sedaghat, C., Mash, D. C., Dietrich, W. D. and Keane, R. W. (2014) RIG-1 receptor expression in the pathology of Alzheimer's disease. J Neuroinflammation, 11, 67.
63. Villa, A., Vegeto, E., Poletti, A. and Maggi, A. (2016) Estrogens, Neuroinflammation, and Neurodegeneration. Endocrine Reviews, 37, 372-402.
64. Chakrabarti, M., Haque, A., Banik, N. L., Nagarkatti, P., Nagarkatti, M. and Ray, S. K. (2014) Estrogen receptor agonists for attenuation of neuroinflammation and neurodegeneration. Brain Research Bulletin, 109, 22-31.
65. Dhandapani, K. M. and Brann, D. W. (2002) Estrogen-astrocyte interactions: implications for neuroprotection. BMC Neurosci, 3, 6.
66. Bacher, M., Deuster, O., Aljabari, B., Egensperger, R., Neff, F., Jessen, F., Popp, J., Noelker, C., Reese, J. P., A1-Abed, Y., et al. (2010) The Role of Macrophage Migration Inhibitory Factor in Alzheimer's Disease. Mol Med, 16, 116-121.
67. David, J. R. (1966) Delayed hypersensitivity in vitro: its mediation by cell-free substances formed by lymphoid cell-antigen interaction. Proc Natl Acad Sci USA, 56, 72-77.
68. Bloom, B. R. and Bennett, B. (1966) Mechanism of a reaction in vitro associated with delayed-type hypersensitivity. Science, 153, 80-82.
69. Cox, G. M., Kithcart, A. P., Pitt, D., Guan, Z., Alexander, J., Williams, J. L., Shawler, T., Dagia, N. M., Popovich, P. G., Satoskar, A. R., et al. (2013) Macrophage migration inhibitory factor potentiates autoimmune-mediated neuroinflammation. J Immunol, 191, 1043-1054.
70. Bernhagen, J., Krohn, R., Lue, H., Gregory, J. L., Zernecke, A., Koenen, R. R., Dewor, M., Georgiev, I., Schober, A., Leng, L., et al. (2007) MIF is a noncognate ligand of CXC chemokine receptors in inflammatory and atherogenic cell recruitment. Nat Med, 13, 587-596.
71. Oliveros, G., Wallace, C. H., Chaudry, O., Liu, Q., Qiu, Y., Xie, L., Rockwell, P., Figueiredo-Pereira, M. E. and Serrano, P. A. (2023) Repurposing ibudilast to mitigate Alzheimer's disease by targeting inflammation. Brain, 146, 898-911.
72. Angelopoulou, E., Pyrgelis, E.-S. and Piperi, C. (2022) Emerging Potential of the Phosphodiesterase (PDE) Inhibitor Ibudilast for Neurodegenerative Diseases: An Update on Preclinical and Clinical Evidence. Molecules, 27, 8448.
73. Wang, H., Mei, Z. lin, Zhong, K. L., Hu, M., Long, Y., Miao, M. X., Li, N., Yan, T. H. and Hong, H. (2014) Pretreatment with antiasthmatic drug ibudilast ameliorates Aβ1-42-induced memory impairment and neurotoxicity in mice. Pharmacology Biochemistry and Behavior, 124, 373-379.
74. Fukui, R., Saitoh, S.-I., Kanno, A., Onji, M., Shibata, T., Ito, A., Onji, M., Matsumoto, M., Akira, S., Yoshida, N., et al. (2011) Unc93B1 restricts systemic lethal inflammation by orchestrating Tolllike receptor 7 and 9 trafficking. Immunity, 35, 69-81.
75. Lee, B. L., Moon, J. E., Shu, J. H., Yuan, L., Newman, Z. R., Schekman, R. and Barton, G. M. (2013) UNC93B1 mediates differential trafficking of endosomal TLRs. eLife, 2, e00291.
76. Pelka, K., Bertheloot, D., Reimer, E., Phulphagar, K., Schmidt, S. V., Christ, A., Stahl, R., Watson, N., Miyake, K., Hacohen, N., et al. (2018) The Chaperone UNC93B1 Regulates Toll-like Receptor Stability Independently of Endosomal TLR Transport. Immunity, 48, 911-922.e7.
77. Klammer, M. G., Dzaye, O., Wallach, T., Kruger, C., Gaessler, D., Buonfiglioli, A., Derkow, K., Kettenmann, H., Brinkmann, M. M. and Lehnardt, S. (2021) UNC93B1 Is Widely Expressed in the Murine CNS and Is Required for Neuroinflammation and Neuronal Injury Induced by MicroRNA let-7b. Frontiers in Immunology, 12.
78. Rath, S., Sharma, R., Gupta, R., Ast, T., Chan, C., Durham, T. J., Goodman, R. P., Grabarek, Z., Haas, M. E., Hung, W. H. W., et al. (2021) MitoCarta3.0: an updated mitochondrial proteome now with suborganelle localization and pathway annotations. Nucleic Acids Res, 49, D1541-D1547.
79. Wu, M., Neilson, A., Swift, A. L., Moran, R., Tamagnine, J., Parslow, D., Armistead, S., Lemire, K., Orrell, J., Teich, J., et al. (2007) Multiparameter metabolic analysis reveals a close link between attenuated mitochondrial bioenergetic function and enhanced glycolysis dependency in human tumor cells. Am J Physiol Cell Physiol, 292, C125-136.
80. Plitzko, B. and Loesgen, S. (2018) Measurement of Oxygen Consumption Rate (OCR) and Extracellular Acidification Rate (ECAR) in Culture Cells for Assessment of the Energy Metabolism. Bio Protoc, 8, e2850.
81. Liddelow, S. A., Guttenplan, K. A., Clarke, L. E., Bennett, F. C., Bohlen, C. J., Schirmer, L., Bennett, M. L., Munch, A. E., Chung, W.-S., Peterson, T. C., et al. (2017) Neurotoxic reactive astrocytes are induced by activated microglia. Nature, 541, 481-487.
82. Guttenplan, K. A., Weigel, M. K., Prakash, P., Wijewardhane, P. R., Hasel, P., Rufen-Blanchette, U., Münch, A. E., Blum, J. A., Fine, J., Neal, M. C., et al. (2021) Neurotoxic reactive astrocytes induce cell death via saturated lipids. Nature, 599, 102-107.
83. Finkel Richard S., Mercuri Eugenio, Darras Basil T., Connolly Anne M., Kuntz Nancy L., Kirschner Janbernd, Chiriboga Claudia A., Saito Kayoko, Servais Laurent, Tizzano Eduardo, et al. (2017) Nusinersen versus Sham Control in Infantile-Onset Spinal Muscular Atrophy. New England Journal of Medicine, 377, 1723-1732.
84. Acsadi, G., Crawford, T. O., Müller-Felber, W., Shieh, P. B., Richardson, R., Natarajan, N., Castro, D., Ramirez-Schrempp, D., Gambino, G., Sun, P., et al. (2021) Safety and efficacy of nusinersen in spinal muscular atrophy: The EMBRACE study. Muscle & Nerve, 63, 668-677.
85. Finkel, R. S., Chiriboga, C. A., Vajsar, J., Day, J. W., Montes, J., De Vivo, D. C., Bishop, K. M., Foster, R., Liu, Y., Ramirez-Schrempp, D., et al. (2021) Treatment of infantile-onset spinal muscular atrophy with nusinersen: final report of a phase 2, open-label, multicentre, dose-escalation study. The Lancet Child & Adolescent Health, 5, 491-500.
86. Frangoul Haydar, Altshuler David, Cappellini M. Domenica, Chen Yi-Shan, Domm Jennifer, Eustace Brenda K., Foell Juergen, de la Fuente Josu, Grupp Stephan, Handgretinger Rupert, et al. (2021) CRISPR-Cas9 Gene Editing for Sickle Cell Disease and β-Thalassemia. New England Journal of Medicine, 384, 252-260.

All publications, patents, and patent applications mentioned in this specification are herein incorporated by reference to the same extent as if each individual publication, patent, or patent application was specifically and individually indicated to be incorporated by reference. In the event that the definition of a term incorporated by reference conflicts with a term defined herein, this specification shall control.

1. A method to modulate expression of APOE in a cell comprising increasing or decreasing expression of APOE-activating noncoding RNA (AANCR) in said cells to increase or decrease APOE expression.

2. The method of claim 1, wherein expression of AANCR is decreased.

3. The method of claim 1, wherein expression AANCR is decreased by contacting said cell with an AANCR inhibitory nucleic acid and/or a small molecule inhibitor to decrease APOE expression, including APOE4 expression and optionally RNA modalities that directly inhibit/knock down APOE4 expression.

4. The method of claim 3, wherein the inhibitory nucleic acid/RNA modality is an dsRNA, siRNA, shRNA, miRNA, ami-RNA, antisense oligonucleotide, gapmer antisense oligonucleotide, or an RNA aptamer.

5. The method of claim 3, wherein the inhibitory nucleic acid binds AANCR.

6. The method of claim 3, wherein the inhibitory nucleic acid binds within the 5′ portion, such as within the first 950 nucleic acids, of AANCR DNA or RNA.

7. The method of claim 4, wherein the inhibitory nucleic acid binds within the following sequence GCGTGGCCTGGGGTCGCTATCTTCCCATCCGGAACATCTGCCCTGCTGGG GGACACTACGGGCCTTCCCTTiCCTGAGGGAGGGTCTCAAGGTCACTTG CCCCCAGCTTGACCTGGCCGGAGTGGCTATAGAGGACTTTGTCCCTGCAG ACTGCAGCAGCAGAGATGACACTGTCTCTGAGTGCAGAGATGGGGGCAGG GAGCTGGGAGAGGGTTCAAGCTACTGGAACAGCTTCAGAACAACTAGGGT ACTAGGAACTGCTGTGTCAGGGAGAAGGGGCTCAAGGACTCGCAGGCCTG GGAGGAGGGGCCTAGGCCA (SEQ ID NO: 46) or the corresponding transcribed eRNA sequence.

8. The method of claim 4, wherein the siRNA is one or more of
siAANCR #1
Sense:
(SEQ IDN NO: 47)
CCAUGCUCAAUACACGUUAtt
Antisense:
(SEQ IDN NO: 48)
UAACGUGUAUUGAGCAUGGag,
siAANCR #2
Sense:
(SEQ IDN NO: 49)
GGAUUUAUAACAGGGCUUAtt
Antisense:
(SEQ IDN NO: 50)
UAAGCCCUGUUAUAAAUCCca,
siAANCR #3
Sense:
(SEQ IDN NO: 51)
UCCUAACCUUAACCCAGAAtt
Antisense:
(SEQ IDN NO: 52)
UUCUGGGUUAAGGUUAGGAtt,
siRNA #4
Antisense
(SEQ IDN NO: 53)
CTGTCTCTGAGTGCAGAGATG
or at least 90% identity thereto.

9. The method of claim 3, wherein the small molecule inhibitor stabilizes the R-loop in AANCR, induces an RNA abasic site in R-loop in AANCR, pauses the RNA Pol II transcription of AANCR and/or inhibits AANCR-APOE interaction.

10. The method of claim 3, wherein the small molecule inhibitor comprises SR11302, TPA, STC-15 or METTL3/14 inhibitors (STM2457).

11. The method of claim 1, wherein expression of AANCR is increased which yields an increase in APOE expression.

12. The method of claim 11, wherein expression of AANCR is increased by contacting said cell with AANCR RNA, an expression vector coding for AANCR RNA or exposing the said cell to stress (such as environmental stress).

13. The method of claim 12, wherein expression of AANCR is increased by exposing said cell to stress.

14. A method to protect a cell against apoptosis during stress comprising contacting said cell with AANCR RNA or an expression vector coding for AANCR RNA.

15. The method of claim 13, wherein the stress is osmotic, amyloid beta aggregate exposure, mitochondrial stress (such as decouplers (antimycin)), heavy metal and/or heat.

16. The method of claim 1, wherein said cell is an astrocyte, macrophage, microglia, oligodendrocyte, endothelial, T-cell, B cell, fibroblast, renal cell, hepatocyte or neuron.

17. The method of claim 16, wherein the renal cell is a proximal tubule cell or a collecting duct cell.

18. A method to treat Alzheimer's Disease in a subject in need thereof comprising administering to said subject an AANCR inhibitory nucleic acid or a small molecule that inhibits full AANCR transcription.

19. The method of claim 18, wherein the subject has one or two copies of APOE4.

20. A method to treat brain injury/disease or kidney disease in a subject in need thereof comprising administering to said subject an AANCR inhibitory nucleic acid or a small molecule that inhibits full AANCR transcription.

21. The method of claim 20, wherein the brain injury/disease is stroke, Parkinson's, caused or aggravated by viral infection (APOE genotype has been documented to severity of HIV, COVID, hepatitis C, herpes simplex and/or influenza) or cardiovascular disease (AANCR-APOE2).

22. The method of claim 18, wherein the inhibitory nucleic acid is an dsRNA, siRNA, shRNA, miRNA, ami-RNA, antisense oligonucleotide, gapmer antisense oligonucleotide, or an RNA aptamer.

23. The method of claim 18, wherein the inhibitory nucleic acid binds AANCR.

24. The method of claim 18, wherein the inhibitory nucleic acid binds within the 5′ portion, such as within the first 950 nucleic acids, of AANCR DNA or RNA.

25. The method of claim 22, wherein the siRNA comprises one or more of
siAANCR #1
Sense:
(SEQ IDN NO: 47)
CCAUGCUCAAUACACGUUAtt
Antisense:
(SEQ IDN NO: 48)
UAACGUGUAUUGAGCAUGGag,
siAANCR #2
Sense:
(SEQ IDN NO: 49)
GGAUUUAUAACAGGGCUUAtt
Antisense:
(SEQ IDN NO: 50)
UAAGCCCUGUUAUAAAUCCca,
siAANCR #3
Sense:
(SEQ IDN NO: 51)
UCCUAACCUUAACCCAGAAtt
Antisense:
(SEQ IDN NO: 52)
UUCUGGGUUAAGGUUAGGAtt,
siRNA #4
Antisense
(SEQ IDN NO: 53)
CTGTCTCTGAGTGCAGAGATG
or at least 90% identity thereto.

26. The method of claim 18 wherein the small molecule inhibitor stabilizes the R-loop in AANCR, induces an RNA abasic site in R-loop in AANCR, pauses the RNA PolII transcription of AANCR and/or inhibits AANCR-APOE interaction.

27. A method to treat cell stress in a subject in need thereof comprising administering to said subject AANCR RNA or an expression vector coding for AANCR RNA.

28. The method of claim 27, wherein the stress is osmotic cell stress.

29. The method of claim 27, wherein the subject has osmotic demyelination syndrome.

30. The method of claim 12, wherein administration is by catheter, surgical placement, or injection.


SEQUENCE LISTING (53 sequences)

SEQ ID NO: 1 - RNA, 2118 nt, Homo sapiens
  source 1..2118: mol_type = other RNA
gaaaaaaaacgatgggaggcctccgttttctcaagtgtgtctggcctgttttgagcatttcatccggagtctggccgccctgaccttcccccagccgcctgcagggggcgccagagggccggagcacggaaagcagcggatccttgatgctgccttaagtccggctcagaggggcgcagcgtggcctggggtcgctatcttcccatccggaacatctgccctgctgggggacactacgggccttcccttgcctgagggtagggtctcaaggtcacttgcccccagcttgacctggccggagtggctatagaggactttgtccctgcagactgcagcagcagagatgacactgtctctgagtgcagagatgggggcagggagctgggagagggttcaagctactggaacagcttcagaacaactagggtactaggaactgctgtgtcagggagaaggggctcaaggactcgcaggcctgggaggaggggcctaggccagccatgggagttgggtcacctgtgtctgaggacttggtgctgtctggattttgccaacctagggctggggtcagctgatgcccaccacgactcccgagcctccaggaactgaaaccctgtctgcccccagggtctggggaaggaggctgctgagtagaaccaaccccaggttaccaaccccacctcagccaccccttgccagccaaagcaaacaggcccggcccggcactgggggttccttctcgaaccaggagttcagcctcccctgacccgcagaatcttctgatcccacccgctccaggagccaggaatgagtcccagtctctcccagttctcactgtgtggttttgccattcgtcttgctgctgaaccacgggtttctcctctgaaacatctgggatttataacagggcttaggaaagtgacagcgtctgagcgttcactgtggcctgtccattgctagccctaacataggaccgctgtgtgccagggctgtcctccatgctcaatacacgttagcttgtcaccaaacatacccgtgccgctgctttcccagtctgatgagcaaaggaacttgatgctcagagaggacaagtcatttgcccaaggtcacacagctggcaactggcagagccaggattcacgccctggcaatttgactccagaatcctaaccttaacccagaagcacggcttcaagcccctggaaaccacaatacctgtggcagccagggggaggtgctggaatctcatttcacatgtggggagggggctcccctgtgctcaaggtcacaaccaaagaggaagctgtgattaaaacccaggtcccatttgcaaagcctcgacttttagcaggtgcatcatactgttcccacccctcccatcccacttctgtccagccgcctagccccactttcttttttttctttttttgagacagtctccctcttgctgaggctggagtgcagtggcgagatctcggctcactgtaacctccgcctcccgggttcaagcgattctcctgcctcagcctcccaagtagctaggattacaggcgcccgccaccacgcctggctaacttttgtatttttagtagagatggggtttcaccatgttggccaggctggtctcaaactcctgaccttaagtgattcgcccactgtggcctcccaaagtgctgggattacaggcgtgagctaccgcccccagcccctcccatcccacttctgtccagccccctagccctactttctttctgggatccaggagtccagatccccagccccctctccagattacattcatccaggcacaggaaaggacagggtcaggaaaggaggactctgggcggcagcctccacattccccttccacgcttggcccccagaatggaggagggtgtctgtattactgggcgaggtgtcctcccttcctggggactgtggggggtggtcaaaagacctctatgccccacctccttcctccctctgccctgctgtgcctggggcagggggagaacagcccacctcgtgactgggggctggcccagcccgccctatccctgggggagggggcgggacagggggagccctataattggacaagtctgggatccttgagtc

SEQ ID NO: 2 - RNA, 2267 nt, Mus musculus
  source 1..2267: mol_type = genomic RNA
tctgggaggcctcggttttctccagttgttgacatggttgccagcaggcggcgccgagggttcagaaagcacagcagcaccaagcagtttagagaaagcttggccaggcatggtcacaccctctgctcttggggacttacactgccggaacatcagaggcctggcctcaccaggagggtggctccaggtcactcgcctctgtgcagtctggccccgagagctggcagaggactttgtctctgtaaacagggtgggggcagggagacggggctcaggagcctccctgtcccaaaacgggctgaggtggtagcttgtgctgactttctcccagtgggaaggtcagaggtctcagaaacttcaggaagaaacggagttcctggaagttcagcacaataaggaaagttactctgggaggaacgggccctagcaccttcccaggctgaaacaggaaatgtcaagttgttttgttttttaaattttgttgaagatgtagctctgggcctcacgtgtcaggcaggtgctgctgggttccctatagtgcttttccgtgaggcctgctctggggtgtgcgataagggcctcagattgggctctgcatctcactgctgcacctcatggcatcccaagggaagcaaagactcacgatgaactggtcagtgtcctagaccgcagcactaagaccctctctactgcctgggctggagatgggagctggcccttagcaacccatgaaaatcatctcaccacacccagtccttgccagtgttttctgaagccaaagctaacaggcctgggcctggccaggcaccctgtacgtacccttggagccaggtgttccgcctctgcccatcctgcagaatcatgttttgccgtgtctggtgccaaacactgctatgtggcttctctcctgccatcaacagctgggaacagggaaccttgtgcaggcagtgcttctagcaagcttgctgtggtctctgagccccttgtcctacctgacttcccaggtacaatggctttcccactttttgggggttttgttgttgttgggtttttttttgtttttcaagacagggtttctctgtgtagtcctgtcggtcctggaactcactttgtagaccaggctggcctccaactcagaaattcacctgcctctgcctccaagtgctgggattatgggattaaaggcatgtgccaccacgcccagctggcttttccactttttagccaggacttcattctattacctgagctcgggatcttcctgcctcagctttgcatatggctagcactatagacccatgttccagtgaatgacttatggcttgtctttttttttttttttttttttttttatgtgcattagtgttttgcctgcatgtatgccttcgtgagggtagcagatcttggtgttacagttgtgagctgctgtgtgggtgctgcgattttgaacctaggtcctgtgaaatgcagtcagtgctcctaacctctgagtcatctctccagctcctgctcttctgcttttatgaggaaaaagaaaagagaagtggcttgagagtggaaaatgcacatgcaggggtgcacacctgcagtcccagcatgctacagcagaggcagaaggacctttgtgggttagagggcagcctgagaatcttatctcaaaacaactttttaaaatgtgctctgtaggggtagctcttccctcccaaggtgacacatctggcaatcgccagaaacagatcaggagcatcaacgcttggtttcccagggcttggcttaatgtatggcttcaaacccatcgggagccaccactgaacagctcctgaaggaactggagcacgtcccagccttggaatggaaagagttcacctgtggtggaggaatcaacaacgagggatcccagaacaacgatcttcaccccagaagctgagcctcttagcccccacccacccatttccatttaggctgccagctcttttctttacaatgcaccagaccccgcggggaaagggaaggagcggttctcagtgccccagtaccaaggcctggattattcaatgaggtgtccgctccctttgttggcgggggaggggagcggggggtcacaaggcatccaaactccacctctttcctctgccctgctgtgaagggggagagaacaacccgcctcgtgacagggggctggcacagcccgccctagccctgaggagggggcgggacagggggagtcctataattggaccggtctgggatccgatcccctgctcaga

SEQ ID NO: 3 - DNA, 20 bp, synthetic construct
  source 1..20: mol_type = other DNA
caggtcacccaggaactgag

SEQ ID NO: 4 - DNA, 19 bp, synthetic construct
  source 1..19: mol_type = other DNA
tccagttccgatttgtagg

SEQ ID NO: 5 - DNA, 22 bp, synthetic construct
  source 1..22: mol_type = other DNA
tcctaaccttaacccagaagca

SEQ ID NO: 6 - DNA, 22 bp, synthetic construct
  source 1..22: mol_type = other DNA
tgggttttaatcacagcttcct

SEQ ID NO: 7 - DNA, 19 bp, synthetic construct
  source 1..19: mol_type = other DNA
cctcccatcccacttctgt

SEQ ID NO: 8 - DNA, 20 bp, synthetic construct
  source 1..20: mol_type = other DNA
agctacttgggaggctgagg

SEQ ID NO: 9 - DNA, 20 bp, synthetic construct
  source 1..20: mol_type = other DNA
caatgaccccttcattgacc

SEQ ID NO: 10 - DNA, 20 bp, synthetic construct
  source 1..20: mol_type = other DNA
gacaagcttcccgttctcag

SEQ ID NO: 11 - DNA, 24 bp, synthetic construct
  source 1..24: mol_type = other DNA
ttatggacaggactgaacgtcttg

SEQ ID NO: 12 - DNA, 21 bp, synthetic construct
  source 1..21: mol_type = other DNA
ccagcaggtcagcaaagaatt

SEQ ID NO: 13 - DNA, 23 bp, synthetic construct
  source 1..23: mol_type = other DNA
aagactctggagagttctgagca

SEQ ID NO: 14 - DNA, 22 bp, synthetic construct
  source 1..22: mol_type = other DNA
ctcggcttttataagtcgtcgt

SEQ ID NO: 15 - RNA, 37 nt, synthetic construct
  source 1..37: mol_type = other RNA
aaataccactacctttatgtttctttacttattcaat

SEQ ID NO: 16 - DNA, 38 bp, synthetic construct
  source 1..38: mol_type = other DNA
attgaataagtaaagaaactataaaggtagtggtattt

SEQ ID NO: 17 - DNA, 37 bp, synthetic construct
  source 1..37: mol_type = other DNA
attgaataagtaaagaaacttaaaggtagtggtattt

SEQ ID NO: 18 - RNA, 38 nt, synthetic construct
  source 1..38: mol_type = other RNA
aaataccactacctttatagtttctttacttattcaat

SEQ ID NO: 19 - RNA, 38 nt, synthetic construct
  source 1..38: mol_type = other RNA
attgaataagtaaagaaactataaaggtagtggtattt

SEQ ID NO: 20 - DNA, 38 bp, synthetic construct
  source 1..38: mol_type = other DNA
aaataccactacctttatagtttctttacttattcaat

SEQ ID NO: 21 - RNA, 37 nt, synthetic construct
  source 1..37: mol_type = other RNA
aaataccactacctttactagtttctttacttacaat

SEQ ID NO: 22 - DNA, 38 bp, synthetic construct; identical to SEQ ID NO: 16
  source 1..38: mol_type = other DNA

SEQ ID NO: 23 - RNA, 26 nt, synthetic construct
  source 1..26: mol_type = other RNA
gatcgtcggactgtagaactctgaac

SEQ ID NO: 24 - RNA, 21 nt, synthetic construct
  source 1..21: mol_type = other RNA
ccttggcacccgagaattcca

SEQ ID NO: 25 - DNA, 50 bp, synthetic construct
  source 1..50: mol_type = other DNA
aatgatacggcgaccaccgagatctacacgttcagagttctacagtccga

SEQ ID NO: 26 - DNA, 20 bp, synthetic construct
  source 1..20: mol_type = other DNA
agtcatttgcccaaggtcac

SEQ ID NO: 27 - DNA, 20 bp, synthetic construct
  source 1..20: mol_type = other DNA
tccccacatgtgaaatgaga

SEQ ID NO: 28 - DNA, 20 bp, synthetic construct
  source 1..20: mol_type = other DNA
caggtcacccaggaactgag

SEQ ID NO: 29 - DNA, 20 bp, synthetic construct
  source 1..20: mol_type = other DNA
tccagttccgatttgtaggc

SEQ ID NO: 30 - RNA, 21 nt, synthetic construct
  source 1..21: mol_type = other RNA
ccatgctcaatacacgttatt

SEQ ID NO: 31 - RNA, 21 nt, synthetic construct
  source 1..21: mol_type = other RNA
ggatttataacagggcttatt

SEQ ID NO: 32 - RNA, 21 nt, synthetic construct
  source 1..21: mol_type = other RNA
tcctaaccttaacccagaatt

SEQ ID NO: 33 - RNA, 23 nt, synthetic construct
  source 1..23: mol_type = other RNA
catctctgcactcagagacagtt

SEQ ID NO: 34 - RNA, 21 nt, synthetic construct
  source 1..21: mol_type = other RNA
ggagttgaaggcctacaaatt

SEQ ID NO: 35 - RNA, 21 nt, synthetic construct
  source 1..21: mol_type = other RNA
gacaatcactgaacgccgatt

SEQ ID NO: 36 - RNA, 21 nt, synthetic construct
  source 1..21: mol_type = other RNA
ctagtttaataaagattcatt

SEQ ID NO: 37 - RNA, 21 nt, synthetic construct
  source 1..21: mol_type = other RNA
aagcggtggagacagagcctt

SEQ ID NO: 38 - RNA, 21 nt, synthetic construct
  source 1..21: mol_type = other RNA
taacgtgtattgagcatggag

SEQ ID NO: 39 - RNA, 21 nt, synthetic construct
  source 1..21: mol_type = other RNA
taagccctgttataaatccca

SEQ ID NO: 40 - RNA, 21 nt, synthetic construct
  source 1..21: mol_type = other RNA
ttctgggttaaggttaggatt

SEQ ID NO: 41 - RNA, 23 nt, synthetic construct
  source 1..23: mol_type = other RNA
ctgtctctgagtgcagagatgtt

SEQ ID NO: 42 - RNA, 21 nt, synthetic construct
  source 1..21: mol_type = other RNA
tttgtaggccttcaactcctt

SEQ ID NO: 43 - RNA, 21 nt, synthetic construct
  source 1..21: mol_type = other RNA
tcggcgttcagtgattgtcgc

SEQ ID NO: 44 - RNA, 21 nt, synthetic construct
  source 1..21: mol_type = other RNA
tgaatctttattaaactaggg

SEQ ID NO: 45 - RNA, 21 nt, synthetic construct
  source 1..21: mol_type = other RNA
ggctctgtctccaccgctttt

SEQ ID NO: 46 - DNA, 319 bp, synthetic construct
  source 1..319: mol_type = other DNA
gcgtggcctggggtcgctatcttcccatccggaacatctgccctgctgggggacactacgggccttcccttgcctgagggtagggtctcaaggtcacttgcccccagcttgacctggccggagtggctatagaggactttgtccctgcagactgcagcagcagagatgacactgtctctgagtgcagagatgggggcagggagctgggagagggttcaagctactggaacagcttcagaacaactagggtactaggaactgctgtgtcagggagaaggggctcaaggactcgcaggcctgggaggaggggcctaggcca

SEQ ID NO: 47 - RNA, 21 nt, synthetic construct
  source 1..21: mol_type = other RNA
ccatgctcaatacacgttatt

SEQ ID NO: 48 - RNA, 21 nt, synthetic construct
  source 1..21: mol_type = other RNA
taacgtgtattgagcatggag

SEQ ID NO: 49 - RNA, 21 nt, synthetic construct
  source 1..21: mol_type = other RNA
ggatttataacagggcttatt

SEQ ID NO: 50 - RNA, 21 nt, synthetic construct
  source 1..21: mol_type = other RNA
taagccctgttataaatccca

SEQ ID NO: 51 - RNA, 21 nt, synthetic construct
  source 1..21: mol_type = other RNA
tcctaaccttaacccagaatt

SEQ ID NO: 52 - RNA, 21 nt, synthetic construct
  source 1..21: mol_type = other RNA
ttctgggttaaggttaggatt

SEQ ID NO: 53 - RNA, 21 nt, synthetic construct
  source 1..21: mol_type = other RNA
ctgtctctgagtgcagagatg